SEQUENCE LISTING (256 sequences)

SEQ ID NO: 1 - skipped

SEQ ID NO: 2 - protein, 9 aa, synthetic construct
RRRKRSVDT

SEQ ID NO: 3 - protein, 9 aa, synthetic construct
RRRKRAVDT

SEQ ID NO: 4 - protein, 9 aa, synthetic construct
RRRKRSLDT

SEQ ID NO: 5 - protein, 9 aa, synthetic construct
RRRKRALDT

SEQ ID NO: 6 - protein, 9 aa, synthetic construct
RRRKRSVST

SEQ ID NO: 7 - protein, 9 aa, synthetic construct
RRRKRAVST

SEQ ID NO: 8 - protein, 9 aa, synthetic construct
RRRKRALST

SEQ ID NO: 9 - protein, 9 aa, synthetic construct
RRRKRSLST

SEQ ID NO: 10 - protein, 9 aa, synthetic construct
RRRKRSVDL

SEQ ID NO: 11 - protein, 9 aa, synthetic construct
RRRKRAVDL

SEQ ID NO: 12 - protein, 9 aa, synthetic construct
RRRKRSLDL

SEQ ID NO: 13 - protein, 9 aa, synthetic construct
RRRKRALDL

SEQ ID NO: 14 - protein, 9 aa, synthetic construct
RRRKRSVSL

SEQ ID NO: 15 - protein, 9 aa, synthetic construct
RRRKRAVSL

SEQ ID NO: 16 - protein, 9 aa, synthetic construct
RRRKRALSL

SEQ ID NO: 17 - protein, 9 aa, synthetic construct
RRRKRSLSL

SEQ ID NO: 18-19 - skipped (2 entries)

SEQ ID NO: 20 - protein, 19 aa, synthetic construct
ATNFSLLKQAGDVEENPGP

SEQ ID NO: 21 - protein, 24 aa, synthetic construct
APVKQTLNFDLLKLAGDVESNPGP

SEQ ID NO: 22 - protein, 20 aa, synthetic construct
QCTNYALLKLAGDVESNPGP

SEQ ID NO: 23 - protein, 18 aa, synthetic construct
EGRGSLLTCGDVEENPGP

SEQ ID NO: 24 - protein, 17 aa, synthetic construct
EGRGSLLTCGDVEENPG

SEQ ID NO: 25 - protein, 5 aa, synthetic construct
ATNFS

SEQ ID NO: 26 - protein, 5 aa, synthetic construct
PTNFS

SEQ ID NO: 27 - protein, 5 aa, synthetic construct
GTNFS

SEQ ID NO: 28 - protein, 5 aa, synthetic construct
AGNFS

SEQ ID NO: 29 - protein, 5 aa, synthetic construct
APNFS

SEQ ID NO: 30 - protein, 5 aa, synthetic construct
ATPFS

SEQ ID NO: 31 - protein, 5 aa, synthetic construct
ATAFS

SEQ ID NO: 32 - protein, 5 aa, synthetic construct
ATNPS

SEQ ID NO: 33 - protein, 5 aa, synthetic construct
ATNAS

SEQ ID NO: 34 - protein, 5 aa, synthetic construct
ATNFP

SEQ ID NO: 35 - DNA, 27 bp, synthetic construct
  source 1..27: mol_type = other DNA
cgtagacggaaacgcagcgttgacacc

SEQ ID NO: 36 - DNA, 27 bp, synthetic construct
  source 1..27: mol_type = other DNA
cgtagacggaaacgcgccgttgacacc

SEQ ID NO: 37 - DNA, 27 bp, synthetic construct
  source 1..27: mol_type = other DNA
cgtagacggaaacgcagcctcgacacc

SEQ ID NO: 38 - DNA, 27 bp, synthetic construct
  source 1..27: mol_type = other DNA
cgtagacggaaacgcgccctcgacacc

SEQ ID NO: 39 - DNA, 27 bp, synthetic construct
  source 1..27: mol_type = other DNA
cgtagacggaaacgcagcgtttctacc

SEQ ID NO: 40 - DNA, 27 bp, synthetic construct
  source 1..27: mol_type = other DNA
cgtagacggaaacgcgccgtttctacc

SEQ ID NO: 41 - DNA, 27 bp, synthetic construct
  source 1..27: mol_type = other DNA
cgtagacggaaacgcgccctctctacc

SEQ ID NO: 42 - DNA, 27 bp, synthetic construct
  source 1..27: mol_type = other DNA
cgtagacggaaacgctccctctctacc

SEQ ID NO: 43 - DNA, 27 bp, synthetic construct
  source 1..27: mol_type = other DNA
cgtagacggaaacgcagcgttgacctc

SEQ ID NO: 44 - DNA, 27 bp, synthetic construct
  source 1..27: mol_type = other DNA
cgtagacggaaacgcgccgttgacctc

SEQ ID NO: 45 - DNA, 27 bp, synthetic construct
  source 1..27: mol_type = other DNA
cgtagacggaaacgcagcctcgacctc

SEQ ID NO: 46 - DNA, 27 bp, synthetic construct
  source 1..27: mol_type = other DNA
cgtagacggaaacgcgccctcgacctc

SEQ ID NO: 47 - DNA, 27 bp, synthetic construct
  source 1..27: mol_type = other DNA
cgtagacggaaacgcagcgtttctctc

SEQ ID NO: 48 - DNA, 27 bp, synthetic construct
  source 1..27: mol_type = other DNA
cgtagacggaaacgcgccgtttctctc

SEQ ID NO: 49 - DNA, 27 bp, synthetic construct
  source 1..27: mol_type = other DNA
cgtagacggaaacgcgccctctctctc

SEQ ID NO: 50 - DNA, 27 bp, synthetic construct
  source 1..27: mol_type = other DNA
cgtagacggaaacgctctctctctctc

SEQ ID NO: 51 - DNA, 57 bp, synthetic construct
  source 1..57: mol_type = other DNA
gctactaacttcagcctgctgaagcaggctggagacgtggaggagaaccctgggccc

SEQ ID NO: 52 - DNA, 72 bp, synthetic construct
  source 1..72: mol_type = other DNA
gcaccggtgaaacagactttgaattttgaccttctgaagttggcaggagacgttgagtccaaccctgggccc

SEQ ID NO: 53 - DNA, 60 bp, synthetic construct
  source 1..60: mol_type = other DNA
cagtgtactaattatgctctcttgaaattggctggagatgttgagagcaaccctgggccc

SEQ ID NO: 54 - DNA, 54 bp, synthetic construct
  source 1..54: mol_type = other DNA
gagggcagaggaagtctgctaacatgcggtgacgtcgaggagaatcctgggccc

SEQ ID NO: 55 - DNA, 51 bp, synthetic construct
  source 1..51: mol_type = other DNA
gagggcagaggaagtctgctaacatgcggtgacgtcgaggagaatcctggg

SEQ ID NO: 56 - DNA, 15 bp, synthetic construct
  source 1..15: mol_type = other DNA
gctactaacttcagc

SEQ ID NO: 57 - DNA, 15 bp, synthetic construct
  source 1..15: mol_type = other DNA
cctactaacttcagc

SEQ ID NO: 58 - DNA, 15 bp, synthetic construct
  source 1..15: mol_type = other DNA
ggcactaacttcagc

SEQ ID NO: 59 - DNA, 15 bp, synthetic construct
  source 1..15: mol_type = other DNA
gctggcaacttcagc

SEQ ID NO: 60 - DNA, 15 bp, synthetic construct
  source 1..15: mol_type = other DNA
gctcctaacttcagc

SEQ ID NO: 61 - DNA, 15 bp, synthetic construct
  source 1..15: mol_type = other DNA
gctactcctttcagc

SEQ ID NO: 62 - DNA, 15 bp, synthetic construct
  source 1..15: mol_type = other DNA
gctactgctttcagc

SEQ ID NO: 63 - DNA, 15 bp, synthetic construct
  source 1..15: mol_type = other DNA
gctactaaccctagc

SEQ ID NO: 64 - DNA, 15 bp, synthetic construct
  source 1..15: mol_type = other DNA
gctactaacgctagc

SEQ ID NO: 65 - DNA, 15 bp, synthetic construct
  source 1..15: mol_type = other DNA
gctactaacttccct

SEQ ID NO: 66 - protein, 9 aa, synthetic construct
PRRKRSVDT

SEQ ID NO: 67 - protein, 9 aa, synthetic construct
GRRKRSVDT

SEQ ID NO: 68 - protein, 9 aa, synthetic construct
ARRKRSVDT

SEQ ID NO: 69 - protein, 9 aa, synthetic construct
KRRKRSVDT

SEQ ID NO: 70 - protein, 9 aa, synthetic construct
NRRKRSVDT

SEQ ID NO: 71 - protein, 9 aa, synthetic construct
DRRKRSVDT

SEQ ID NO: 72 - protein, 9 aa, synthetic construct
CRRKRSVDT

SEQ ID NO: 73 - protein, 9 aa, synthetic construct
ERRKRSVDT

SEQ ID NO: 74 - protein, 9 aa, synthetic construct
QRRKRSVDT

SEQ ID NO: 75 - protein, 9 aa, synthetic construct
HRRKRSVDT

SEQ ID NO: 76 - protein, 9 aa, synthetic construct
IRRKRSVDT

SEQ ID NO: 77 - protein, 9 aa, synthetic construct
LRRKRSVDT

SEQ ID NO: 78 - protein, 9 aa, synthetic construct
MRRKRSVDT

SEQ ID NO: 79 - protein, 9 aa, synthetic construct
FRRKRSVDT

SEQ ID NO: 80 - protein, 9 aa, synthetic construct
SRRKRSVDT

SEQ ID NO: 81 - protein, 9 aa, synthetic construct
TRRKRSVDT

SEQ ID NO: 82 - protein, 9 aa, synthetic construct
WRRKRSVDT

SEQ ID NO: 83 - protein, 9 aa, synthetic construct
YRRKRSVDT

SEQ ID NO: 84 - protein, 9 aa, synthetic construct
VRRKRSVDT

SEQ ID NO: 85 - protein, 9 aa, synthetic construct
RRRKRPVDT

SEQ ID NO: 86 - protein, 9 aa, synthetic construct
RRRKRGVDT

SEQ ID NO: 87 - protein, 9 aa, synthetic construct
RRRKRAVDT

SEQ ID NO: 88 - protein, 9 aa, synthetic construct
RRRKRRVDT

SEQ ID NO: 89 - protein, 9 aa, synthetic construct
RRRKRKVDT

SEQ ID NO: 90 - protein, 9 aa, synthetic construct
RRRKRNVDT

SEQ ID NO: 91 - protein, 9 aa, synthetic construct
RRRKRDVDT

SEQ ID NO: 92 - protein, 9 aa, synthetic construct
RRRKRCVDT

SEQ ID NO: 93 - protein, 9 aa, synthetic construct
RRRKREVDT

SEQ ID NO: 94 - protein, 9 aa, synthetic construct
RRRKRQVDT

SEQ ID NO: 95 - protein, 9 aa, synthetic construct
RRRKRHVDT

SEQ ID NO: 96 - protein, 9 aa, synthetic construct
RRRKRIVDT

SEQ ID NO: 97 - protein, 9 aa, synthetic construct
RRRKRLVDT

SEQ ID NO: 98 - protein, 9 aa, synthetic construct
RRRKRMVDT

SEQ ID NO: 99 - protein, 9 aa, synthetic construct
RRRKRFVDT

SEQ ID NO: 100 - protein, 9 aa, synthetic construct
RRRKRTVDT

SEQ ID NO: 101 - protein, 9 aa, synthetic construct
RRRKRWVDT

SEQ ID NO: 102 - protein, 9 aa, synthetic construct
RRRKRYVDT

SEQ ID NO: 103 - protein, 9 aa, synthetic construct
RRRKRVVDT

SEQ ID NO: 104 - protein, 9 aa, synthetic construct
RRRKRSPDT

SEQ ID NO: 105 - protein, 9 aa, synthetic construct
RRRKRSGDT

SEQ ID NO: 106 - protein, 9 aa, synthetic construct
RRRKRSADT

SEQ ID NO: 107 - protein, 9 aa, synthetic construct
RRRKRSRDT

SEQ ID NO: 108 - protein, 9 aa, synthetic construct
RRRKRSKDT

SEQ ID NO: 109 - protein, 9 aa, synthetic construct
RRRKRSNDT

SEQ ID NO: 110 - protein, 9 aa, synthetic construct
RRRKRSDDT

SEQ ID NO: 111 - protein, 9 aa, synthetic construct
RRRKRSCDT

SEQ ID NO: 112 - protein, 9 aa, synthetic construct
RRRKRSEDT

SEQ ID NO: 113 - protein, 9 aa, synthetic construct
RRRKRSQDT

SEQ ID NO: 114 - protein, 9 aa, synthetic construct
RRRKRSHDT

SEQ ID NO: 115 - protein, 9 aa, synthetic construct
RRRKRSIDT

SEQ ID NO: 116 - protein, 9 aa, synthetic construct
RRRKRSLDT

SEQ ID NO: 117 - protein, 9 aa, synthetic construct
RRRKRSMDT

SEQ ID NO: 118 - protein, 9 aa, synthetic construct
RRRKRSFDT

SEQ ID NO: 119 - protein, 9 aa, synthetic construct
RRRKRSSDT

SEQ ID NO: 120 - protein, 9 aa, synthetic construct
RRRKRSTDT

SEQ ID NO: 121 - protein, 9 aa, synthetic construct
RRRKRSWDT

SEQ ID NO: 122 - protein, 9 aa, synthetic construct
RRRKRSYDT

SEQ ID NO: 123 - protein, 9 aa, synthetic construct
RRRKRSVPT

SEQ ID NO: 124 - protein, 9 aa, synthetic construct
RRRKRSVGT

SEQ ID NO: 125 - protein, 9 aa, synthetic construct
RRRKRSVAT

SEQ ID NO: 126 - protein, 9 aa, synthetic construct
RRRKRSVRT

SEQ ID NO: 127 - protein, 9 aa, synthetic construct
RRRKRSVKT

SEQ ID NO: 128 - protein, 9 aa, synthetic construct
RRRKRSVNT

SEQ ID NO: 129 - protein, 9 aa, synthetic construct
RRRKRSVCT

SEQ ID NO: 130 - protein, 9 aa, synthetic construct
RRRKRSVET

SEQ ID NO: 131 - protein, 9 aa, synthetic construct
RRRKRSVQT

SEQ ID NO: 132 - protein, 9 aa, synthetic construct
RRRKRSVHT

SEQ ID NO: 133 - protein, 9 aa, synthetic construct
RRRKRSVIT

SEQ ID NO: 134 - protein, 9 aa, synthetic construct
RRRKRSVLT

SEQ ID NO: 135 - protein, 9 aa, synthetic construct
RRRKRSVMT

SEQ ID NO: 136 - protein, 9 aa, synthetic construct
RRRKRSVFT

SEQ ID NO: 137 - protein, 9 aa, synthetic construct
RRRKRSVST

SEQ ID NO: 138 - protein, 9 aa, synthetic construct
RRRKRSVTT

SEQ ID NO: 139 - protein, 9 aa, synthetic construct
RRRKRSVWT

SEQ ID NO: 140 - protein, 9 aa, synthetic construct
RRRKRSVYT

SEQ ID NO: 141 - protein, 9 aa, synthetic construct
RRRKRSVVT

SEQ ID NO: 142 - protein, 9 aa, synthetic construct
RRRKRSVDP

SEQ ID NO: 143 - protein, 9 aa, synthetic construct
RRRKRSVDG

SEQ ID NO: 144 - protein, 9 aa, synthetic construct
RRRKRSVDA

SEQ ID NO: 145 - protein, 9 aa, synthetic construct
RRRKRSVDR

SEQ ID NO: 146 - protein, 9 aa, synthetic construct
RRRKRSVDK

SEQ ID NO: 147 - protein, 9 aa, synthetic construct
RRRKRSVDN

SEQ ID NO: 148 - protein, 9 aa, synthetic construct
RRRKRSVDD

SEQ ID NO: 149 - protein, 9 aa, synthetic construct
RRRKRSVDC

SEQ ID NO: 150 - protein, 9 aa, synthetic construct
RRRKRSVDE

SEQ ID NO: 151 - protein, 9 aa, synthetic construct
RRRKRSVDQ

SEQ ID NO: 152 - protein, 9 aa, synthetic construct
RRRKRSVDH

SEQ ID NO: 153 - protein, 9 aa, synthetic construct
RRRKRSVDI

SEQ ID NO: 154 - protein, 9 aa, synthetic construct
RRRKRSVDL

SEQ ID NO: 155 - protein, 9 aa, synthetic construct
RRRKRSVDM

SEQ ID NO: 156 - protein, 9 aa, synthetic construct
RRRKRSVDF

SEQ ID NO: 157 - protein, 9 aa, synthetic construct
RRRKRSVDS

SEQ ID NO: 158 - protein, 9 aa, synthetic construct
RRRKRSVDW

SEQ ID NO: 159 - protein, 9 aa, synthetic construct
RRRKRSVDY

SEQ ID NO: 160 - protein, 9 aa, synthetic construct
RRRKRSVDV

SEQ ID NO: 161 - DNA, 27 bp, synthetic construct
  source 1..27: mol_type = other DNA
cctagacggaaacgcagcgttgacacc

SEQ ID NO: 162 - DNA, 27 bp, synthetic construct
  source 1..27: mol_type = other DNA
ggcagacggaaacgcagcgttgacacc

SEQ ID NO: 163 - DNA, 27 bp, synthetic construct
  source 1..27: mol_type = other DNA
gccagacggaaacgcagcgttgacacc

SEQ ID NO: 164 - DNA, 27 bp, synthetic construct
  source 1..27: mol_type = other DNA
aagagacggaaacgcagcgttgacacc

SEQ ID NO: 165 - DNA, 27 bp, synthetic construct
  source 1..27: mol_type = other DNA
aacagacggaaacgcagcgttgacacc

SEQ ID NO: 166 - DNA, 27 bp, synthetic construct
  source 1..27: mol_type = other DNA
gacagacggaaacgcagcgttgacacc

SEQ ID NO: 167 - DNA, 27 bp, synthetic construct
  source 1..27: mol_type = other DNA
tgtagacggaaacgcagcgttgacacc

SEQ ID NO: 168 - DNA, 27 bp, synthetic construct
  source 1..27: mol_type = other DNA
gagagacggaaacgcagcgttgacacc

SEQ ID NO: 169 - DNA, 27 bp, synthetic construct
  source 1..27: mol_type = other DNA
cagagacggaaacgcagcgttgacacc

SEQ ID NO: 170 - DNA, 27 bp, synthetic construct
  source 1..27: mol_type = other DNA
cacagacggaaacgcagcgttgacacc

SEQ ID NO: 171 - DNA, 27 bp, synthetic construct
  source 1..27: mol_type = other DNA
atcagacggaaacgcagcgttgacacc

SEQ ID NO: 172 - DNA, 27 bp, synthetic construct
  source 1..27: mol_type = other DNA
ctgagacggaaacgcagcgttgacacc

SEQ ID NO: 173 - DNA, 27 bp, synthetic construct
  source 1..27: mol_type = other DNA
atgagacggaaacgcagcgttgacacc

SEQ ID NO: 174 - DNA, 27 bp, synthetic construct
  source 1..27: mol_type = other DNA
ttcagacggaaacgcagcgttgacacc

SEQ ID NO: 175 - DNA, 27 bp, synthetic construct
  source 1..27: mol_type = other DNA
tccagacggaaacgcagcgttgacacc

SEQ ID NO: 176 - DNA, 27 bp, synthetic construct
  source 1..27: mol_type = other DNA
accagacggaaacgcagcgttgacacc

SEQ ID NO: 177 - DNA, 27 bp, synthetic construct
  source 1..27: mol_type = other DNA
tggagacggaaacgcagcgttgacacc

SEQ ID NO: 178 - DNA, 27 bp, synthetic construct
  source 1..27: mol_type = other DNA
tacagacggaaacgcagcgttgacacc

SEQ ID NO: 179 - DNA, 27 bp, synthetic construct
  source 1..27: mol_type = other DNA
gtgagacggaaacgcagcgttgacacc

SEQ ID NO: 180 - DNA, 27 bp, synthetic construct
  source 1..27: mol_type = other DNA
agaagacggaaacgccctgttgacacc

SEQ ID NO: 181 - DNA, 27 bp, synthetic construct
  source 1..27: mol_type = other DNA
agaagacggaaacgcggcgttgacacc

SEQ ID NO: 182 - DNA, 27 bp, synthetic construct
  source 1..27: mol_type = other DNA
agaagacggaaacgcgccgttgacacc

SEQ ID NO: 183 - DNA, 27 bp, synthetic construct
  source 1..27: mol_type = other DNA
agaagacggaaacgcagagttgacacc

SEQ ID NO: 184 - DNA, 27 bp, synthetic construct
  source 1..27: mol_type = other DNA
agaagacggaaacgcaaggttgacacc

SEQ ID NO: 185 - DNA, 27 bp, synthetic construct
  source 1..27: mol_type = other DNA
agaagacggaaacgcaacgttgacacc

SEQ ID NO: 186 - DNA, 27 bp, synthetic construct
  source 1..27: mol_type = other DNA
agaagacggaaacgcgacgttgacacc

SEQ ID NO: 187 - DNA, 27 bp, synthetic construct
  source 1..27: mol_type = other DNA
agaagacggaaacgctgcgttgacacc

SEQ ID NO: 188 - DNA, 27 bp, synthetic construct
  source 1..27: mol_type = other DNA
agaagacggaaacgcgaggttgacacc

SEQ ID NO: 189 - DNA, 27 bp, synthetic construct
  source 1..27: mol_type = other DNA
agaagacggaaacgccaggttgacacc

SEQ ID NO: 190 - DNA, 27 bp, synthetic construct
  source 1..27: mol_type = other DNA
agaagacggaaacgccacgttgacacc

SEQ ID NO: 191 - DNA, 27 bp, synthetic construct
  source 1..27: mol_type = other DNA
agaagacggaaacgcatcgttgacacc

SEQ ID NO: 192 - DNA, 27 bp, synthetic construct
  source 1..27: mol_type = other DNA
agaagacggaaacgcctcgttgacacc

SEQ ID NO: 193 - DNA, 27 bp, synthetic construct
  source 1..27: mol_type = other DNA
agaagacggaaacgcatggttgacacc

SEQ ID NO: 194 - DNA, 27 bp, synthetic construct
  source 1..27: mol_type = other DNA
agaagacggaaacgcttcgttgacacc

SEQ ID NO: 195 - DNA, 27 bp, synthetic construct
  source 1..27: mol_type = other DNA
agaagacggaaacgcaccgttgacacc

SEQ ID NO: 196 - DNA, 27 bp, synthetic construct
  source 1..27: mol_type = other DNA
agaagacggaaacgctgggttgacacc

SEQ ID NO: 197 - DNA, 27 bp, synthetic construct
  source 1..27: mol_type = other DNA
agaagacggaaacgctacgttgacacc

SEQ ID NO: 198 - DNA, 27 bp, synthetic construct
  source 1..27: mol_type = other DNA
agaagacggaaacgcgttgttgacacc

SEQ ID NO: 199 - DNA, 27 bp, synthetic construct
  source 1..27: mol_type = other DNA
agaagacggaaacgcagccctgacacc

SEQ ID NO: 200 - DNA, 27 bp, synthetic construct
  source 1..27: mol_type = other DNA
agaagacggaaacgcagcggcgacacc

SEQ ID NO: 201 - DNA, 27 bp, synthetic construct
  source 1..27: mol_type = other DNA
agaagacggaaacgcagcgccgacacc

SEQ ID NO: 202 - DNA, 27 bp, synthetic construct
  source 1..27: mol_type = other DNA
agaagacggaaacgcagcagagacacc

SEQ ID NO: 203 - RNA, 27 nt, synthetic construct
  source 1..27: mol_type = other RNA
agaagacggaaacgcagcaaggacacc

SEQ ID NO: 204 - DNA, 27 bp, synthetic construct
  source 1..27: mol_type = other DNA
agaagacggaaacgcagcaacgacacc

SEQ ID NO: 205 - DNA, 27 bp, synthetic construct
  source 1..27: mol_type = other DNA
agaagacggaaacgcagcgacgacacc

SEQ ID NO: 206 - DNA, 27 bp, synthetic construct
  source 1..27: mol_type = other DNA
agaagacggaaacgcagctgcgacacc

SEQ ID NO: 207 - DNA, 27 bp, synthetic construct
  source 1..27: mol_type = other DNA
agaagacggaaacgcagcgaggacacc

SEQ ID NO: 208 - DNA, 27 bp, synthetic construct
  source 1..27: mol_type = other DNA
agaagacggaaacgcagccaggacacc

SEQ ID NO: 209 - DNA, 27 bp, synthetic construct
  source 1..27: mol_type = other DNA
agaagacggaaacgcagccacgacacc

SEQ ID NO: 210 - DNA, 27 bp, synthetic construct
  source 1..27: mol_type = other DNA
agaagacggaaacgcagcatcgacacc

SEQ ID NO: 211 - DNA, 27 bp, synthetic construct
  source 1..27: mol_type = other DNA
agaagacggaaacgcagcctcgacacc

SEQ ID NO: 212 - DNA, 27 bp, synthetic construct
  source 1..27: mol_type = other DNA
agaagacggaaacgcagcatggacacc

SEQ ID NO: 213 - DNA, 27 bp, synthetic construct
  source 1..27: mol_type = other DNA
agaagacggaaacgcagcttcgacacc

SEQ ID NO: 214 - DNA, 27 bp, synthetic construct
  source 1..27: mol_type = other DNA
agaagacggaaacgcagcagcgacacc

SEQ ID NO: 215 - DNA, 27 bp, synthetic construct
  source 1..27: mol_type = other DNA
agaagacggaaacgcagcaccgacacc

SEQ ID NO: 216 - DNA, 27 bp, synthetic construct
  source 1..27: mol_type = other DNA
agaagacggaaacgcagctgggacacc

SEQ ID NO: 217 - DNA, 27 bp, synthetic construct
  source 1..27: mol_type = other DNA
agaagacggaaacgcagctacgacacc

SEQ ID NO: 218 - DNA, 27 bp, synthetic construct
  source 1..27: mol_type = other DNA
agaagacggaaacgcagcgttcctacc

SEQ ID NO: 219 - DNA, 27 bp, synthetic construct
  source 1..27: mol_type = other DNA
agaagacggaaacgcagcgttggcacc

SEQ ID NO: 220 - DNA, 27 bp, synthetic construct
  source 1..27: mol_type = other DNA
agaagacggaaacgcagcgttgccacc

SEQ ID NO: 221 - DNA, 27 bp, synthetic construct
  source 1..27: mol_type = other DNA
agaagacggaaacgcagcgttagaacc

SEQ ID NO: 222 - DNA, 27 bp, synthetic construct
  source 1..27: mol_type = other DNA
agaagacggaaacgcagcgttaagacc

SEQ ID NO: 223 - DNA, 27 bp, synthetic construct
  source 1..27: mol_type = other DNA
agaagacggaaacgcagcgttaacacc

SEQ ID NO: 224 - DNA, 27 bp, synthetic construct
  source 1..27: mol_type = other DNA
agaagacggaaacgcagcgtttgcacc

SEQ ID NO: 225 - DNA, 27 bp, synthetic construct
  source 1..27: mol_type = other DNA
agaagacggaaacgcagcgttgagacc

SEQ ID NO: 226 - DNA, 27 bp, synthetic construct
  source 1..27: mol_type = other DNA
agaagacggaaacgcagcgttcagacc

SEQ ID NO: 227 - DNA, 27 bp, synthetic construct
  source 1..27: mol_type = other DNA
agaagacggaaacgcagcgttcacacc

SEQ ID NO: 228 - DNA, 27 bp, synthetic construct
  source 1..27: mol_type = other DNA
agaagacggaaacgcagcgttatcacc

SEQ ID NO: 229 - DNA, 27 bp, synthetic construct
  source 1..27: mol_type = other DNA
agaagacggaaacgcagcgttctcacc

SEQ ID NO: 230 - DNA, 27 bp, synthetic construct
  source 1..27: mol_type = other DNA
agaagacggaaacgcagcgttatgacc

SEQ ID NO: 231 - DNA, 27 bp, synthetic construct
  source 1..27: mol_type = other DNA
agaagacggaaacgcagcgttttcacc

SEQ ID NO: 232 - DNA, 27 bp, synthetic construct
  source 1..27: mol_type = other DNA
agaagacggaaacgcagcgttagcacc

SEQ ID NO: 233 - DNA, 27 bp, synthetic construct
  source 1..27: mol_type = other DNA
agaagacggaaacgcagcgttaccacc

SEQ ID NO: 234 - DNA, 27 bp, synthetic construct
  source 1..27: mol_type = other DNA
agaagacggaaacgcagcgtttggacc

SEQ ID NO: 235 - DNA, 27 bp, synthetic construct
  source 1..27: mol_type = other DNA
agaagacggaaacgcagcgtttacacc

SEQ ID NO: 236 - DNA, 27 bp, synthetic construct
  source 1..27: mol_type = other DNA
agaagacggaaacgcagcgttgttacc

SEQ ID NO: 237 - DNA, 27 bp, synthetic construct
  source 1..27: mol_type = other DNA
agaagacggaaacgcagcgttgaccct

SEQ ID NO: 238 - DNA, 27 bp, synthetic construct
  source 1..27: mol_type = other DNA
agaagacggaaacgcagcgttgacggc

SEQ ID NO: 239 - DNA, 27 bp, synthetic construct
  source 1..27: mol_type = other DNA
agaagacggaaacgcagcgttgacgcc

SEQ ID NO: 240 - DNA, 27 bp, synthetic construct
  source 1..27: mol_type = other DNA
agaagacggaaacgcagcgttgacaga

SEQ ID NO: 241 - DNA, 27 bp, synthetic construct
  source 1..27: mol_type = other DNA
agaagacggaaacgcagcgttgacaag

SEQ ID NO: 242 - DNA, 27 bp, synthetic construct
  source 1..27: mol_type = other DNA
agaagacggaaacgcagcgttgacaac

SEQ ID NO: 243 - DNA, 27 bp, synthetic construct
  source 1..27: mol_type = other DNA
agaagacggaaacgcagcgttgacgac

SEQ ID NO: 244 - DNA, 27 bp, synthetic construct
  source 1..27: mol_type = other DNA
agaagacggaaacgcagcgttgactgc

SEQ ID NO: 245 - DNA, 27 bp, synthetic construct
  source 1..27: mol_type = other DNA
agaagacggaaacgcagcgttgacgag

SEQ ID NO: 246 - DNA, 27 bp, synthetic construct
  source 1..27: mol_type = other DNA
agaagacggaaacgcagcgttgaccag

SEQ ID NO: 247 - DNA, 27 bp, synthetic construct
  source 1..27: mol_type = other DNA
agaagacggaaacgcagcgttgaccac

SEQ ID NO: 248 - DNA, 27 bp, synthetic construct
  source 1..27: mol_type = other DNA
agaagacggaaacgcagcgttgacatc

SEQ ID NO: 249 - DNA, 27 bp, synthetic construct
  source 1..27: mol_type = other DNA
agaagacggaaacgcagcgttgacctc

SEQ ID NO: 250 - DNA, 27 bp, synthetic construct
  source 1..27: mol_type = other DNA
agaagacggaaacgcagcgttgacatg

SEQ ID NO: 251 - DNA, 27 bp, synthetic construct
  source 1..27: mol_type = other DNA
agaagacggaaacgcagcgttgacttc

SEQ ID NO: 252 - DNA, 27 bp, synthetic construct
  source 1..27: mol_type = other DNA
agaagacggaaacgcagcgttgacagc

SEQ ID NO: 253 - DNA, 27 bp, synthetic construct
  source 1..27: mol_type = other DNA
agaagacggaaacgcagcgttgactgg

SEQ ID NO: 254 - DNA, 27 bp, synthetic construct
  source 1..27: mol_type = other DNA
agaagacggaaacgcagcgttgactac

SEQ ID NO: 255 - DNA, 27 bp, synthetic construct
  source 1..27: mol_type = other DNA
agaagacggaaacgcagcgttgacgtt

SEQ ID NO: 256 - skipped

(19) United States
(12) Patent Application Publication
(10) Pub. No.: US 2026/0242821 A1
(43) Pub. Date: Aug. 20, 2026

(54) ENGINEERED FURIN CLEAVAGE SEQUENCES FOR CO-EXPRESSION OF MULTIPLE GENES IN MAMMALIAN CELLS

(71) Applicant: AGENCY FOR SCIENCE, TECHNOLOGY AND RESEARCH, Singapore (SG)

(72) Inventors: Yuansheng YANG, Singapore (SG); Han Kee ONG, Singapore (SG); Mariati MARIATI, Singapore (SG); Tran Bich Ngan NGUYEN, Singapore (SG); Xuezhi BI, Singapore (SG); Pei Zhen Jessica NG, Singapore (SG)

(21) Appl. No.: 19/161,813

(22) PCT Filed: Mar. 6, 2024
(86) PCT No.: PCT/SG2024/050135

§ 371 Date: Sep. 3, 2025

(30) Foreign Application Priority Data
Mar. 6, 2023 (SG) 10202300591X

(51) Int. Cl. C12N 15/85; C07K 16/46

(52) U.S. Cl. CPC C12N 15/85; C07K 16/46; C07K 2319/50; C07K 2319/912; C12N 2840/203

(57) ABSTRACT

The present disclosure relates to an expression system for an antigen binding molecule. In particular, the present disclosure relates to an expression system comprising plasmid vectors for co-expression of multiple genes, such as but not limited to the light chain and heavy chain of antibodies at high levels and homogeneously across cells.

### CROSS-REFERENCE TO RELATED APPLICATIONS

This application claims the benefit of priority of SG provisional application No. 10202300591X, filed on 6 Mar. 2023, the contents of it being hereby incorporated by reference in its entirety for all purposes.

### TECHNICAL FIELD

The present disclosure relates to, but is not limited to an expression system for an antigen binding molecule. In particular, the present disclosure relates to an expression system comprising plasmid vectors for co-expression of multiple genes, such as but not limited to the light chain and heavy chain of antibodies at high levels and homogeneously across cells.

### BACKGROUND

The display of full length of monoclonal antibodies (mAbs) on the surface of mammalian cells requires tightly coupled co-expression of light chain and heavy chain genes in one single vector, and is an important aspect of various biotechnological applications such as antibody production and development. Conventionally, achieving the surface display of mAbs on mammalian cells has involved the use of diverse molecular tools, with multiple promoters (MP) and internal ribosome entry site (IRES) strategies.

Co-expression of light chain and heavy chain of mAbs using MP and IRES results in either low antibody expression or heterogenous antibody expression across transfected cells. Therefore, there is a need to develop an expression system that addresses and mitigates these limitations, for enhanced secretion of antibodies with the correct size without compromising the level of secreted antibody titers and homogeneity of antibody display. There is a need to provide an improved expression system comprising plasmid vectors for co-expression of multiple genes, such as the light chain and heavy chain of mAbs at high levels and homogeneously across cells.

### SUMMARY

In one aspect, the present disclosure refers to an expression system for an antigen binding molecule, comprising:
a first polynucleotide encoding a first part of the antigen binding molecule;
a second polynucleotide encoding a first cleavage site comprising an amino acid sequence Z-R-X1-X2-R-X3-X4-X5-X6 (SEQ ID NO: 256), wherein the second polynucleotide is located downstream of the first polynucleotide;
a third polynucleotide encoding a second cleavage site comprising a 2A polypeptide or a fragment thereof, wherein the third polynucleotide is located downstream of the second polynucleotide; and
a fourth polynucleotide encoding a second part of the antigen binding molecule, wherein the fourth polynucleotide is located downstream of the third polynucleotide,
wherein when the first and second cleavage sites are cleaved, an antigen binding molecule comprising the first and second parts of the antigen binding molecule is released,
wherein Z is R, P, G, A, K, N, Q, H, I, L, M, F, S, T, W, Y, or V, and
wherein X1, X2, X3, X4, X5 and X6 is R, P, G, A, D, C, E, K, N, Q, H, I, L, M, F, S, T, W, Y, or V.

In one aspect, the present disclosure refers to an expression system for an antigen binding molecule, wherein the antigen binding molecule is either secretable or membrane-bound, comprising:
a first polynucleotide encoding a first part of the antigen binding molecule;
a second polynucleotide encoding a first cleavage site comprising an amino acid sequence Z-R-X1-X2-R-X3-X4-X5-X6 (SEQ ID NO: 256), wherein the second polynucleotide is located downstream of the first polynucleotide;
a third polynucleotide encoding a second cleavage site comprising a 2A polypeptide or a fragment thereof, wherein the third polynucleotide is located downstream of the second polynucleotide;
a fourth polynucleotide encoding a second part of the antigen binding molecule, wherein the fourth polynucleotide is located downstream of the third polynucleotide;
a fifth polynucleotide encoding a third cleavage site comprising a Furin cleavage sequence variant comprising:
   (i) a Furin consensus sequence R-X-K-R (SEQ ID NO: 18); or
   (ii) a Furin consensus sequence R-X-R-R (SEQ ID NO: 19),
wherein the fifth polynucleotide is located downstream of the fourth polynucleotide; and
a sixth polynucleotide encoding a membrane anchor polypeptide, wherein the sixth polynucleotide is located downstream of the fifth polynucleotide;
wherein when the first, second and third cleavage sites are cleaved, a secretable antigen binding molecule comprising the first and second parts of the antigen binding molecule is released;
wherein when the first and second cleavage sites are cleaved and the third cleavage site is not cleaved, a membrane-bound antigen binding molecule comprising the first and second parts of the antigen binding molecule, the third cleavage site, and the membrane anchor polypeptide is released,
wherein Z is R, P, G, A, K, N, Q, H, I, L, M, F, S, T, W, Y, or V,
wherein X1, X2, X3, X4, X5 and X6 is R, P, G, A, D, C, E, K, N, Q, H, I, L, M, F, S, T, W, Y, or V, and
wherein X is any amino acid.

In one aspect, the present disclosure refers to a vector comprising the expression system disclosed herein.

In one aspect, the present disclosure refers to a host cell comprising the expression system disclosed herein or the vector disclosed herein.

In one aspect, the present disclosure refers to a kit comprising the expression system disclosed herein, the vector disclosed herein, or the host cell disclosed herein.

In one aspect, the present disclosure refers to the expression system disclosed herein, the vector disclosed herein, the host cell disclosed herein, or the kit disclosed herein for use in screening antibody libraries or antibody production.

In one aspect, the present disclosure refers to a method of producing one or more secretable antigen binding molecules and/or one or more membrane-bound antigen binding molecules, comprising culturing the host cell disclosed herein under suitable culture conditions such that one or more secretable antigen binding molecules and/or one or more membrane-bound antigen binding molecules are produced.

In one aspect, the present disclosure refers to a method for detecting the presence of one or more secretable antigen binding molecules and/or one or more membrane-bound antigen binding molecules, the method comprising:
providing an expression system disclosed herein;
delivering said expression system to one or more target cells;
wherein said target cells transcribe said expression system into one or more amino acid sequences,
wherein when all of the cleavage sites in the one or more amino acid sequences are cleaved, one or more secretable antigen binding molecules comprising the first and second parts of the antigen binding molecules are secreted by the target cells,
detecting the presence or absence of the one or more secretable antigen binding molecules and/or one or more membrane-bound antigen binding molecules.

### BRIEF DESCRIPTION OF DRAWINGS

The disclosure will be better understood with reference to the detailed description when considered in conjunction with the non-limiting examples and the accompanying drawings, in which:

FIG. 1 (comprised of FIGS. 1A and 1B) is an overview of the recombinase-mediated cassette exchange (RMCE) strategy and the design of targeting vectors for co-expression of light chain and heavy chain of mAbs. FIG. 1A is a schematic overview of a CHO targeted integration platform for high throughput screening of antibody libraries. FIG. 1B is a schematic representation of targeting vectors for IRES, MP and 2A peptides co-expression of light chain and heavy chain of mAbs in the context of the CHO targeted integration platform. hCMV, human cytomegalovirus major (CMV) major immediate-early gene enhancer and promoter; mCMV, murine CMV enhancer and promoter; ChiP, chimeric promoter consisting of mCMV enhancer, human CMV major immediate-early gene promoter and intron A; Flpe, enhanced flippase; HYGR, cDNA encoding hygromycin resistant gene; (ATG-) Pur, ATG-start codon removed puromycin resistant gene; F and F3, wild-type and mutated flippase recognition targets; pA, SV40 polyadenylation signal; IRES, wild-type encephalomyocarditis virus (EMCV) internal ribosome entry site (IRES); LC, light chain cDNA; HC, heavy chain cDNA; DRS, DNA regulatory elements such as IRES, promoter or FCS-2A peptide; FCS, engineered furin cleavage sequence for simultaneous display and secretion of antibodies; FCSm, minimal furin cleavage sequence, RRKR; FCS1, engineered FCS variant RRKRSVDTS; GPI, DNA encoding the glycosidylphosphatidylinositol membrane anchor derived from human decay-accelerating factor; T2A, 2A peptide derived from Thosea asigna virus.

FIG. 2 (comprised of FIGS. 2A, 2B, 2C, and 2D) shows the various comparative data of cells transfected with IRES, MP, Fm-2A, F1-2A, F2-2A and F3-2A targeting vectors. FIG. 2A is a line chart showing the viability of cells transfected with IRES, MP, Fm-2A, F1-2A, F2-2A and F3-2A targeting vectors. FIG. 2B is a flow cytometry analysis data showing the level of homogeneity of antibody display in stable pool of cells transfected with IRES, MP, Fm-2A, F1-2A, F2-2A and F3-2A targeting vectors. FIG. 2C is a bar chart showing the secreted antibody titers in fed-batch cultures of cells transfected with IRES, MP, Fm-2A, F1-2A, F2-2A and F3-2A targeting vectors. FIG. 2D is an SDS-PAGE gel showing the quality of the secreted antibodies by cells transfected with IRES, MP, Fm-2A, F1-2A, F2-2A and F3-2A targeting vectors, as determined by SDS-PAGE under reducing conditions.

FIG. 3 (comprised of FIGS. 3A, 3B, 3C, 3D, 3E, 3F, and 3G) shows the intact mass spectrometry (MS) analysis data of light chain and heavy chain polypeptides expressed from different targeting vectors. FIG. 3A shows the intact MS analysis data of light chain and heavy chain polypeptides expressed from control (IRES)-mediated targeting vector. FIG. 3B shows the intact MS analysis data of light chain and heavy chain polypeptides expressed from Fm-2A-mediated targeting vector. FIG. 3C shows the intact MS analysis data of light chain and heavy chain polypeptides expressed from F1-2A-mediated targeting vector. FIG. 3D shows the intact MS analysis data of light chain and heavy chain polypeptides expressed from F2-2A-mediated targeting vector. FIG. 3E shows the intact MS analysis data of light chain and heavy chain polypeptides expressed from F3-2A-mediated targeting vector. FIG. 3F shows the zoomed in image for intact MS analysis data of light chain polypeptides expressed from F3-2A-mediated targeting vector. FIG. 3G shows the percentage cleavage efficiency of the Fm, F1, F2, and F3 furin cleavage sequences. The cleavage efficiency of the furin cleavage sequences was determined as the intensity of cleaved light chain species divided by the intensity of total light chain species, including both cleaved light chain and un-cleaved light chain attached with 2A peptide.

FIG. 4 (comprised of FIGS. 4A and 4B) shows the evaluation of cleavage efficiency of 16 FCS variants. FIG. 4A shows the amino acid sequences of the 16 different FCS variants.

FIG. 4B shows the SDS-PAGE analysis of secreted antibodies expressed from targeting vectors containing combinations of different FCS variants and 2A under reducing conditions.

FIG. 5 (comprised of FIGS. 5A, 5B, 5C, 5D, 5E, and 5F) shows the evaluation of cleavage efficiency of 95 FCS variants. FIG. 5A shows the amino acid sequences of the 95 different FCS variants. FIG. 5B shows the SDS-PAGE analysis of secreted antibodies expressed from targeting vectors containing unique mutations at position P5 of furin cleavage variants F19-F37. FIG. 5C shows the SDS-PAGE analysis of secreted antibodies expressed from targeting vectors containing unique mutations at position P1′ of furin cleavage variants F38-F56. FIG. 5D shows the SDS-PAGE analysis of secreted antibodies expressed from targeting vectors containing unique mutations at position P2′ of furin cleavage variants F57-F75. FIG. 5E shows the SDS-PAGE analysis of secreted antibodies expressed from targeting vectors containing unique mutations at position P3′ of furin cleavage variants F76-F94. FIG. 5F shows the SDS-PAGE analysis of secreted antibodies expressed from targeting vectors containing unique mutations at position P4′ of furin cleavage variants F95-F113.

### DETAILED DESCRIPTION

The present disclosure describes an expression system comprising plasmid vectors containing engineered furin cleavage sequences for co-expression of multiple genes, such as light chain and heavy of mAbs at high levels and homogeneously across cells.

In one aspect, the present disclosure refers to an expression system for an antigen binding molecule, comprising:
a first polynucleotide encoding a first part of the antigen binding molecule;
a second polynucleotide encoding a first cleavage site comprising an amino acid sequence Z-R-X1-X2-R-X3-X4-X5-X6 (SEQ ID NO: 256), wherein the second polynucleotide is located downstream of the first polynucleotide;
a third polynucleotide encoding a second cleavage site comprising a 2A polypeptide or a fragment thereof, wherein the third polynucleotide is located downstream of the second polynucleotide; and
a fourth polynucleotide encoding a second part of the antigen binding molecule, wherein the fourth polynucleotide is located downstream of the third polynucleotide,
wherein when the first and second cleavage sites are cleaved, an antigen binding molecule comprising the first and second parts of the antigen binding molecule is released,
wherein Z is R, P, G, A, K, N, Q, H, I, L, M, F, S, T, W, Y, or V, and
wherein X1, X2, X3, X4, X5 and X6 is R, P, G, A, D, C, E, K, N, Q, H, I, L, M, F, S, T, W, Y, or V.

In one example of the preceding aspect, the present disclosure refers to an expression system for an antigen binding molecule, comprising:
a first polynucleotide encoding a first part of the antigen binding molecule;
a second polynucleotide encoding a first cleavage site comprising an amino acid sequence R-R-X1-X2-R-X3-X4-X5-X6 (SEQ ID NO: 1), wherein the second polynucleotide is located downstream of the first polynucleotide;
a third polynucleotide encoding a second cleavage site comprising a 2A polypeptide or a fragment thereof, wherein the third polynucleotide is located downstream of the second polynucleotide; and
a fourth polynucleotide encoding a second part of the antigen binding molecule, wherein the fourth polynucleotide is located downstream of the third polynucleotide, wherein when the first and second cleavage sites are cleaved, an antigen binding molecule comprising the first and second parts of the antigen binding molecule is released.

In one example, the term “expression system” used herein refers to a DNA construct that is designed to produce a protein, or an RNA (ribonucleic acid), either inside or outside a cell. The expression system can exist on its own or be incorporated into a vector. Exemplary expression systems include, but are not limited to, a mammalian expression system, an insect expression system, a yeast expression system, a bacteria expression system, an algae expression system or a cell-free expression system. The expression system can comprise the following components including, but not limited to, a promoter, one or more genes of interest, and one or more identification tags. In one example, the expression system can be used for dual screening purpose to screen for the expression levels of a secretable and/or membrane-bound antigen binding molecule.

In one example, the term “antigen binding molecule” used herein refers to an antibody, or other protein construct, such as a domain. In one example, the antigen binding molecule described herein is an antibody. In one example, the first part of the antigen binding molecule is a light chain of an antibody, or other protein construct. In one example, the antigen binding molecule described herein is a light chain or heavy chain variable region of an antibody. In one example, the antigen binding molecule described herein is a heavy chain variable region or light chain variable region. In one specific example, the antigen binding molecule described herein is a light chain variable region of an antibody.

In one example, the expression system for an antigen binding molecule described herein comprises a first polynucleotide encoding a first part of the antigen binding molecule. In one example, the first polynucleotide encodes a light chain of an antibody. The term “polynucleotide” used herein refers to a nucleotide sequence that encodes for the product of interest, or a fragment, derivative, mutein, or variant thereof. A polynucleotide includes DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. In one example, the polynucleotide may encode a first or second part of the antigen binding molecule, a first or second or third cleavage site, or a membrane anchor polypeptide.

In one example, the expression system for an antigen binding molecule described herein comprises a second polynucleotide encoding a first cleavage site. In one example, the first cleavage site comprises a Furin cleavage sequence (FCS). In one example, the first cleavage site comprises a minimal furin cleavage sequence flanked by one or more additional amino acids, wherein the additional amino acids may be any amino acids. The amino acids may be naturally occurring amino acids or non-naturally occurring amino acids. In one example, the first cleavage site comprises an amino acid sequence Z-R-X1-X2-R-X3-X4-X5-X6 (SEQ ID NO: 256), wherein X1-X6 may be any amino acids. In one example, X1-X6 are naturally occurring amino acids or non-naturally occurring amino acids or a combination thereof. In one example, X1-X6 are naturally occurring amino acids. In one example, the naturally occurring amino acids are Arginine (R), Proline (P), Glycine (G), Alanine (A), Aspartic Acid (D), Cysteine (C), Glutamic Acid (E), Lysine (K), Asparagine (N), Glutamine (Q), Histidine (H), Isoleucine (I), Leucine (L), Methionine (M), Phenylalanine (F), Serine(S), Threonine (T), Tryptophan (W), Tyrosine (Y), or Valine (V). The term “Furin” used herein refers to a ubiquitous subtilisin-like proprotein convertase which cleaves proteins containing its recognition site. The terms, not limited to “Furin consensus sequence”, “minimal cleavage site”, “minimal Furin cleavage consensus sequence” or “Furin recognition site” as used herein, refer to an amino acid sequence of R-X-K-R (SEQ ID NO: 18) or R-X-R-R (SEQ ID NO: 19), wherein X can be any amino acids. In one example, X is a naturally occurring amino acid. In one example, X is Arginine (R). Furin recognizes a protein that comprises a Furin consensus sequence, which leads to the cleavage of the protein that comprises the Furin consensus sequence. The cleavage can take place in, for example, the Golgi. In one example, the amino acid sequence of the first cleavage site comprises an amino acid sequence R-R-X1-X2-R-X3-X4-X5-X6 (SEQ ID NO: 1). In one example, the second polynucleotide encodes a first cleavage site comprising an amino acid sequence R-R-R-K-R-S-V-D-T (SEQ ID NO: 2). In one example, the cleavage site comprising an amino acid sequence R-R-R-K-R-S-V-D-T (SEQ ID NO: 2) corresponds to FCS variant F2. In one example, the second polynucleotide encodes a first cleavage site comprising an amino acid sequence R-R-R-K-R-A-V-D-T (SEQ ID NO: 3). In one example, the cleavage site comprising an amino acid sequence R-R-R-K-R-A-V-D-T (SEQ ID NO: 3) corresponds to FCS variant F4. In one example, the second polynucleotide encodes a first cleavage site comprising an amino acid sequence R-R-R-K-R-S-L-D-T (SEQ ID NO: 4). In one example, the cleavage site comprising an amino acid sequence R-R-R-K-R-S-L-D-T (SEQ ID NO: 4) corresponds to FCS variant F5. In one example, the second polynucleotide encodes a first cleavage site comprising an amino acid sequence R-R-R-K-R-A-L-D-T (SEQ ID NO: 5). In one example, the cleavage site comprising an amino acid sequence R-R-R-K-R-A-L-D-T (SEQ ID NO: 5) corresponds to FCS variant F6. In one example, the second polynucleotide encodes a first cleavage site comprising an amino acid sequence R-R-R-K-R-S-V-S-T (SEQ ID NO: 6). In one example, the cleavage site comprising an amino acid sequence R-R-R-K-R-S-V-S-T (SEQ ID NO: 6) corresponds to FCS variant F7. In one example, the second polynucleotide encodes a first cleavage site comprising an amino acid sequence R-R-R-K-R-A-V-S-T (SEQ ID NO: 7). In one example, the cleavage site comprising an amino acid sequence R-R-R-K-R-A-V-S-T (SEQ ID NO: 7) corresponds to FCS variant F8. In one example, the second polynucleotide encodes a first cleavage site comprising an amino acid sequence R-R-R-K-R-A-L-S-T (SEQ ID NO: 8). In one example, the cleavage site comprising an amino acid sequence R-R-R-K-R-A-L-S-T (SEQ ID NO: 8) corresponds to FCS variant F9. In one example, the second polynucleotide encodes a first cleavage site comprising an amino acid sequence R-R-R-K-R-S-L-S-T (SEQ ID NO: 9). In one example, the cleavage site comprising an amino acid sequence R-R-R-K-R-S-L-S-T (SEQ ID NO: 9) corresponds to FCS variant F10. In one example, the second polynucleotide encodes a first cleavage site comprising an amino acid sequence R-R-R-K-R-S-V-D-L (SEQ ID NO: 10). In one example, the cleavage site comprising an amino acid sequence R-R-R-K-R-S-V-D-L (SEQ ID NO: 10) corresponds to FCS variant F11. In one example, the second polynucleotide encodes a first cleavage site comprising an amino acid sequence R-R-R-K-R-A-V-D-L (SEQ ID NO: 11). In one example, the cleavage site comprising an amino acid sequence R-R-R-K-R-A-V-D-L (SEQ ID NO: 11) corresponds to FCS variant F12. In one example, the second polynucleotide encodes a first cleavage site comprising an amino acid sequence R-R-R-K-R-S-L-D-L (SEQ ID NO: 12). In one example, the cleavage site comprising an amino acid sequence R-R-R-K-R-S-L-D-L (SEQ ID NO: 12) corresponds to FCS variant F13. In one example, the second polynucleotide encodes a first cleavage site comprising an amino acid sequence R-R-R-K-R-A-L-D-L (SEQ ID NO: 13). In one example, the cleavage site comprising an amino acid sequence R-R-R-K-R-A-L-D-L (SEQ ID NO: 13) corresponds to FCS variant F14. In one example, the second polynucleotide encodes a first cleavage site comprising an amino acid sequence R-R-R-K-R-S-V-S-L (SEQ ID NO: 14). In one example, the cleavage site comprising an amino acid sequence R-R-R-K-R-S-V-S-L (SEQ ID NO: 14) corresponds to FCS variant F15. In one example, the second polynucleotide encodes a first cleavage site comprising an amino acid sequence R-R-R-K-R-A-V-S-L (SEQ ID NO: 15). In one example, the cleavage site comprising an amino acid sequence R-R-R-K-R-A-V-S-L (SEQ ID NO: 15) corresponds to FCS variant F16. In one example, the second polynucleotide encodes a first cleavage site comprising an amino acid sequence R-R-R-K-R-A-L-S-L (SEQ ID NO: 16). In one example, the cleavage site comprising an amino acid sequence R-R-R-K-R-A-L-S-L (SEQ ID NO: 16) corresponds to FCS variant F17. In one example, the second polynucleotide encodes a first cleavage site comprising an amino acid sequence R-R-R-K-R-S-L-S-L (SEQ ID NO: 17). In one example, the cleavage site comprising an amino acid sequence R-R-R-K-R-S-L-S-L (SEQ ID NO: 17) corresponds to FCS variant F18. In one example, the second polynucleotide is located downstream of the first polynucleotide. In one example, the second polynucleotide encodes a first cleavage site comprising an amino acid sequence P-R-R-K-R-S-V-D-T (SEQ ID NO: 66). In one example, the cleavage site comprising an amino acid sequence P-R-R-K-R-S-V-D-T (SEQ ID NO: 66) corresponds to FCS variant F19. In one example, the second polynucleotide encodes a first cleavage site comprising an amino acid sequence G-R-R-K-R-S-V-D-T (SEQ ID NO: 67). In one example, the cleavage site comprising an amino acid sequence G-R-R-K-R-S-V-D-T (SEQ ID NO: 67) corresponds to FCS variant F20. In one example, the second polynucleotide encodes a first cleavage site comprising an amino acid sequence A-R-R-K-R-S-V-D-T (SEQ ID NO: 68). In one example, the cleavage site comprising an amino acid sequence A-R-R-K-R-S-V-D-T (SEQ ID NO: 68) corresponds to FCS variant F21. In one example, the second polynucleotide encodes a first cleavage site comprising an amino acid sequence K-R-R-K-R-S-V-D-T (SEQ ID NO: 69). In one example, the cleavage site comprising an amino acid sequence K-R-R-K-R-S-V-D-T (SEQ ID NO: 69) corresponds to FCS variant F22. In one example, the second polynucleotide encodes a first cleavage site comprising an amino acid sequence N-R-R-K-R-S-V-D-T (SEQ ID NO: 70). In one example, the cleavage site comprising an amino acid sequence N-R-R-K-R-S-V-D-T (SEQ ID NO: 70) corresponds to FCS variant F23. In one example, the second polynucleotide encodes a first cleavage site comprising an amino acid sequence D-R-R-K-R-S-V-D-T (SEQ ID NO: 71). In one example, the cleavage site comprising an amino acid sequence D-R-R-K-R-S-V-D-T (SEQ ID NO: 71) corresponds to FCS variant F24. In one example, the second polynucleotide encodes a first cleavage site comprising an amino acid sequence C-R-R-K-R-S-V-D-T (SEQ ID NO: 72). In one example, the cleavage site comprising an amino acid sequence C-R-R-K-R-S-V-D-T (SEQ ID NO: 72) corresponds to FCS variant F25. In one example, the second polynucleotide encodes a first cleavage site comprising an amino acid sequence E-R-R-K-R-S-V-D-T (SEQ ID NO: 73). In one example, the cleavage site comprising an amino acid sequence E-R-R-K-R-S-V-D-T (SEQ ID NO: 73) corresponds to FCS variant F26. In one example, the second polynucleotide encodes a first cleavage site comprising an amino acid sequence Q-R-R-K-R-S-V-D-T (SEQ ID NO: 74). In one example, the cleavage site comprising an amino acid sequence Q-R-R-K-R-S-V-D-T (SEQ ID NO: 74) corresponds to FCS variant F27. In one example, the second polynucleotide encodes a first cleavage site comprising an amino acid sequence H-R-R-K-R-S-V-D-T (SEQ ID NO: 75). In one example, the cleavage site comprising an amino acid sequence H-R-R-K-R-S-V-D-T (SEQ ID NO: 75) corresponds to FCS variant F28. In one example, the second polynucleotide encodes a first cleavage site comprising an amino acid sequence I-R-R-K-R-S-V-D-T (SEQ ID NO: 76). In one example, the cleavage site comprising an amino acid sequence I-R-R-K-R-S-V-D-T (SEQ ID NO: 76) corresponds to FCS variant F29. In one example, the second polynucleotide encodes a first cleavage site comprising an amino acid sequence L-R-R-K-R-S-V-D-T (SEQ ID NO: 77). In one example, the cleavage site comprising an amino acid sequence L-R-R-K-R-S-V-D-T (SEQ ID NO: 77) corresponds to FCS variant F30. In one example, the second polynucleotide encodes a first cleavage site comprising an amino acid sequence M-R-R-K-R-S-V-D-T (SEQ ID NO: 78). In one example, the cleavage site comprising an amino acid sequence M-R-R-K-R-S-V-D-T (SEQ ID NO: 78) corresponds to FCS variant F31. In one example, the second polynucleotide encodes a first cleavage site comprising an amino acid sequence F-R-R-K-R-S-V-D-T (SEQ ID NO: 79). In one example, the cleavage site comprising an amino acid sequence F-R-R-K-R-S-V-D-T (SEQ ID NO: 79) corresponds to FCS variant F32. In one example, the second polynucleotide encodes a first cleavage site comprising an amino acid sequence S-R-R-K-R-S-V-D-T (SEQ ID NO: 80). In one example, the cleavage site comprising an amino acid sequence S-R-R-K-R-S-V-D-T (SEQ ID NO: 80) corresponds to FCS variant F33. In one example, the second polynucleotide encodes a first cleavage site comprising an amino acid sequence T-R-R-K-R-S-V-D-T (SEQ ID NO: 81). In one example, the cleavage site comprising an amino acid sequence T-R-R-K-R-S-V-D-T (SEQ ID NO: 81) corresponds to FCS variant F34. In one example, the second polynucleotide encodes a first cleavage site comprising an amino acid sequence W-R-R-K-R-S-V-D-T (SEQ ID NO: 82). In one example, the cleavage site comprising an amino acid sequence W-R-R-K-R-S-V-D-T (SEQ ID NO: 82) corresponds to FCS variant F35. In one example, the second polynucleotide encodes a first cleavage site comprising an amino acid sequence Y-R-R-K-R-S-V-D-T (SEQ ID NO: 83). In one example, the cleavage site comprising an amino acid sequence Y-R-R-K-R-S-V-D-T (SEQ ID NO: 83) corresponds to FCS variant F36. In one example, the second polynucleotide encodes a first cleavage site comprising an amino acid sequence V-R-R-K-R-S-V-D-T (SEQ ID NO: 84). In one example, the cleavage site comprising an amino acid sequence V-R-R-K-R-S-V-D-T (SEQ ID NO: 84) corresponds to FCS variant F37. In one example, the second polynucleotide encodes a first cleavage site comprising an amino acid sequence R-R-R-K-R-P-V-D-T (SEQ ID NO: 85). In one example, the cleavage site comprising an amino acid sequence R-R-R-K-R-P-V-D-T (SEQ ID NO: 85) corresponds to FCS variant F38. In one example, the second polynucleotide encodes a first cleavage site comprising an amino acid sequence R-R-R-K-R-G-V-D-T (SEQ ID NO: 86). In one example, the cleavage site comprising an amino acid sequence R-R-R-K-R-G-V-D-T (SEQ ID NO: 86) corresponds to FCS variant F39. In one example, the second polynucleotide encodes a first cleavage site comprising an amino acid sequence R-R-R-K-R-A-V-D-T (SEQ ID NO: 87). In one example, the cleavage site comprising an amino acid sequence R-R-R-K-R-A-V-D-T (SEQ ID NO: 87) corresponds to FCS variant F40. In one example, the second polynucleotide encodes a first cleavage site comprising an amino acid sequence R-R-R-K-R-R-V-D-T (SEQ ID NO: 88). In one example, the cleavage site comprising an amino acid sequence R-R-R-K-R-R-V-D-T (SEQ ID NO: 88) corresponds to FCS variant F41. In one example, the second polynucleotide encodes a first cleavage site comprising an amino acid sequence R-R-R-K-R-K-V-D-T (SEQ ID NO: 89). In one example, the cleavage site comprising an amino acid sequence R-R-R-K-R-K-V-D-T (SEQ ID NO: 89) corresponds to FCS variant F42. In one example, the second polynucleotide encodes a first cleavage site comprising an amino acid sequence R-R-R-K-R-N-V-D-T (SEQ ID NO: 90). In one example, the cleavage site comprising an amino acid sequence R-R-R-K-R-N-V-D-T (SEQ ID NO: 90) corresponds to FCS variant F43. In one example, the second polynucleotide encodes a first cleavage site comprising an amino acid sequence R-R-R-K-R-D-V-D-T (SEQ ID NO: 91). In one example, the cleavage site comprising an amino acid sequence R-R-R-K-R-D-V-D-T (SEQ ID NO: 91) corresponds to FCS variant F44. In one example, the second polynucleotide encodes a first cleavage site comprising an amino acid sequence R-R-R-K-R-C-V-D-T (SEQ ID NO: 92). In one example, the cleavage site comprising an amino acid sequence R-R-R-K-R-C-V-D-T (SEQ ID NO: 92) corresponds to FCS variant F45. In one example, the second polynucleotide encodes a first cleavage site comprising an amino acid sequence R-R-R-K-R-E-V-D-T (SEQ ID NO: 93). In one example, the cleavage site comprising an amino acid sequence R-R-R-K-R-E-V-D-T (SEQ ID NO: 93) corresponds to FCS variant F46. In one example, the second polynucleotide encodes a first cleavage site comprising an amino acid sequence R-R-R-K-R-Q-V-D-T (SEQ ID NO: 94). In one example, the cleavage site comprising an amino acid sequence R-R-R-K-R-Q-V-D-T (SEQ ID NO: 94) corresponds to FCS variant F47. In one example, the second polynucleotide encodes a first cleavage site comprising an amino acid sequence R-R-R-K-R-H-V-D-T (SEQ ID NO: 95). In one example, the cleavage site comprising an amino acid sequence R-R-R-K-R-H-V-D-T (SEQ ID NO: 95) corresponds to FCS variant F48. In one example, the second polynucleotide encodes a first cleavage site comprising an amino acid sequence R-R-R-K-R-I-V-D-T (SEQ ID NO: 96). In one example, the cleavage site comprising an amino acid sequence R-R-R-K-R-I-V-D-T (SEQ ID NO: 96) corresponds to FCS variant F49. In one example, the second polynucleotide encodes a first cleavage site comprising an amino acid sequence R-R-R-K-R-L-V-D-T (SEQ ID NO: 97). In one example, the cleavage site comprising an amino acid sequence R-R-R-K-R-L-V-D-T (SEQ ID NO: 97) corresponds to FCS variant F50. In one example, the second polynucleotide encodes a first cleavage site comprising an amino acid sequence R-R-R-K-R-M-V-D-T (SEQ ID NO: 98). In one example, the cleavage site comprising an amino acid sequence R-R-R-K-R-M-V-D-T (SEQ ID NO: 98) corresponds to FCS variant F51. In one example, the second polynucleotide encodes a first cleavage site comprising an amino acid sequence R-R-R-K-R-F-V-D-T (SEQ ID NO: 99). In one example, the cleavage site comprising an amino acid sequence R-R-R-K-R-F-V-D-T (SEQ ID NO: 99) corresponds to FCS variant F52. In one example, the second polynucleotide encodes a first cleavage site comprising an amino acid sequence R-R-R-K-R-T-V-D-T (SEQ ID NO: 100). In one example, the cleavage site comprising an amino acid sequence R-R-R-K-R-T-V-D-T (SEQ ID NO: 100) corresponds to FCS variant F53. In one example, the second polynucleotide encodes a first cleavage site comprising an amino acid sequence R-R-R-K-R-W-V-D-T (SEQ ID NO: 101). In one example, the cleavage site comprising an amino acid sequence R-R-R-K-R-W-V-D-T (SEQ ID NO: 101) corresponds to FCS variant F54. In one example, the second polynucleotide encodes a first cleavage site comprising an amino acid sequence R-R-R-K-R-Y-V-D-T (SEQ ID NO: 102). In one example, the cleavage site comprising an amino acid sequence R-R-R-K-R-Y-V-D-T (SEQ ID NO: 102) corresponds to FCS variant F55. In one example, the second polynucleotide encodes a first cleavage site comprising an amino acid sequence R-R-R-K-R-V-V-D-T (SEQ ID NO: 103). In one example, the cleavage site comprising an amino acid sequence R-R-R-K-R-V-V-D-T (SEQ ID NO: 103) corresponds to FCS variant F56. In one example, the second polynucleotide encodes a first cleavage site comprising an amino acid sequence R-R-R-K-R-S-P-D-T (SEQ ID NO: 104). In one example, the cleavage site comprising an amino acid sequence R-R-R-K-R-S-P-D-T (SEQ ID NO: 104) corresponds to FCS variant F57. In one example, the second polynucleotide encodes a first cleavage site comprising an amino acid sequence R-R-R-K-R-S-G-D-T (SEQ ID NO: 105). In one example, the cleavage site comprising an amino acid sequence R-R-R-K-R-S-G-D-T (SEQ ID NO: 105) corresponds to FCS variant F58. In one example, the second polynucleotide encodes a first cleavage site comprising an amino acid sequence R-R-R-K-R-S-A-D-T (SEQ ID NO: 106). In one example, the cleavage site comprising an amino acid sequence R-R-R-K-R-S-A-D-T (SEQ ID NO: 106) corresponds to FCS variant F59. In one example, the second polynucleotide encodes a first cleavage site comprising an amino acid sequence R-R-R-K-R-S-R-D-T (SEQ ID NO: 107). In one example, the cleavage site comprising an amino acid sequence R-R-R-K-R-S-R-D-T (SEQ ID NO: 107) corresponds to FCS variant F60. In one example, the second polynucleotide encodes a first cleavage site comprising an amino acid sequence R-R-R-K-R-S-K-D-T (SEQ ID NO: 108). In one example, the cleavage site comprising an amino acid sequence R-R-R-K-R-S-K-D-T (SEQ ID NO: 108) corresponds to FCS variant F61. In one example, the second polynucleotide encodes a first cleavage site comprising an amino acid sequence R-R-R-K-R-S-N-D-T (SEQ ID NO: 109). In one example, the cleavage site comprising an amino acid sequence R-R-R-K-R-S-N-D-T (SEQ ID NO: 109) corresponds to FCS variant F62. In one example, the second polynucleotide encodes a first cleavage site comprising an amino acid sequence R-R-R-K-R-S-D-D-T (SEQ ID NO: 110). In one example, the cleavage site comprising an amino acid sequence R-R-R-K-R-S-D-D-T (SEQ ID NO: 110) corresponds to FCS variant F63. In one example, the second polynucleotide encodes a first cleavage site comprising an amino acid sequence R-R-R-K-R-S-C-D-T (SEQ ID NO: 111). In one example, the cleavage site comprising an amino acid sequence R-R-R-K-R-S-C-D-T (SEQ ID NO: 111) corresponds to FCS variant F64. In one example, the second polynucleotide encodes a first cleavage site comprising an amino acid sequence R-R-R-K-R-S-E-D-T (SEQ ID NO: 112). In one example, the cleavage site comprising an amino acid sequence R-R-R-K-R-S-E-D-T (SEQ ID NO: 112) corresponds to FCS variant F65. In one example, the second polynucleotide encodes a first cleavage site comprising an amino acid sequence R-R-R-K-R-S-Q-D-T (SEQ ID NO: 113). In one example, the cleavage site comprising an amino acid sequence R-R-R-K-R-S-Q-D-T (SEQ ID NO: 113) corresponds to FCS variant F66. In one example, the second polynucleotide encodes a first cleavage site comprising an amino acid sequence R-R-R-K-R-S-H-D-T (SEQ ID NO: 114). In one example, the cleavage site comprising an amino acid sequence R-R-R-K-R-S-H-D-T (SEQ ID NO: 114) corresponds to FCS variant F67. In one example, the second polynucleotide encodes a first cleavage site comprising an amino acid sequence R-R-R-K-R-S-I-D-T (SEQ ID NO: 115). In one example, the cleavage site comprising an amino acid sequence R-R-R-K-R-S-I-D-T (SEQ ID NO: 115) corresponds to FCS variant F68. In one example, the second polynucleotide encodes a first cleavage site comprising an amino acid sequence R-R-R-K-R-S-L-D-T (SEQ ID NO: 116). In one example, the cleavage site comprising an amino acid sequence R-R-R-K-R-S-L-D-T (SEQ ID NO: 116) corresponds to FCS variant F69. In one example, the second polynucleotide encodes a first cleavage site comprising an amino acid sequence R-R-R-K-R-S-M-D-T (SEQ ID NO: 117). In one example, the cleavage site comprising an amino acid sequence R-R-R-K-R-S-M-D-T (SEQ ID NO: 117) corresponds to FCS variant F70. In one example, the second polynucleotide encodes a first cleavage site comprising an amino acid sequence R-R-R-K-R-S-F-D-T (SEQ ID NO: 118). In one example, the cleavage site comprising an amino acid sequence R-R-R-K-R-S-F-D-T (SEQ ID NO: 118) corresponds to FCS variant F71. In one example, the second polynucleotide encodes a first cleavage site comprising an amino acid sequence R-R-R-K-R-S-S-D-T (SEQ ID NO: 119). In one example, the cleavage site comprising an amino acid sequence R-R-R-K-R-S-S-D-T (SEQ ID NO: 119) corresponds to FCS variant F72. In one example, the second polynucleotide encodes a first cleavage site comprising an amino acid sequence R-R-R-K-R-S-T-D-T (SEQ ID NO: 120). In one example, the cleavage site comprising an amino acid sequence R-R-R-K-R-S-T-D-T (SEQ ID NO: 120) corresponds to FCS variant F73. In one example, the second polynucleotide encodes a first cleavage site comprising an amino acid sequence R-R-R-K-R-S-W-D-T (SEQ ID NO: 121). In one example, the cleavage site comprising an amino acid sequence R-R-R-K-R-S-W-D-T (SEQ ID NO: 121) corresponds to FCS variant F74. In one example, the second polynucleotide encodes a first cleavage site comprising an amino acid sequence R-R-R-K-R-S-Y-D-T (SEQ ID NO: 122). In one example, the cleavage site comprising an amino acid sequence R-R-R-K-R-S-Y-D-T (SEQ ID NO: 122) corresponds to FCS variant F75. In one example, the second polynucleotide encodes a first cleavage site comprising an amino acid sequence R-R-R-K-R-S-V-P-T (SEQ ID NO: 123). In one example, the cleavage site comprising an amino acid sequence R-R-R-K-R-S-V-P-T (SEQ ID NO: 123) corresponds to FCS variant F76. In one example, the second polynucleotide encodes a first cleavage site comprising an amino acid sequence R-R-R-K-R-S-V-G-T (SEQ ID NO: 124). In one example, the cleavage site comprising an amino acid sequence R-R-R-K-R-S-V-G-T (SEQ ID NO: 124) corresponds to FCS variant F77. In one example, the second polynucleotide encodes a first cleavage site comprising an amino acid sequence R-R-R-K-R-S-V-A-T (SEQ ID NO: 125). In one example, the cleavage site comprising an amino acid sequence R-R-R-K-R-S-V-A-T (SEQ ID NO: 125) corresponds to FCS variant F78. In one example, the second polynucleotide encodes a first cleavage site comprising an amino acid sequence R-R-R-K-R-S-V-R-T (SEQ ID NO: 126). In one example, the cleavage site comprising an amino acid sequence R-R-R-K-R-S-V-R-T (SEQ ID NO: 126) corresponds to FCS variant F79. In one example, the second polynucleotide encodes a first cleavage site comprising an amino acid sequence R-R-R-K-R-S-V-K-T (SEQ ID NO: 127). In one example, the cleavage site comprising an amino acid sequence R-R-R-K-R-S-V-K-T (SEQ ID NO: 127) corresponds to FCS variant F80. In one example, the second polynucleotide encodes a first cleavage site comprising an amino acid sequence R-R-R-K-R-S-V-N-T (SEQ ID NO: 128). In one example, the cleavage site comprising an amino acid sequence R-R-R-K-R-S-V-N-T (SEQ ID NO: 128) corresponds to FCS variant F81. In one example, the second polynucleotide encodes a first cleavage site comprising an amino acid sequence R-R-R-K-R-S-V-C-T (SEQ ID NO: 129). In one example, the cleavage site comprising an amino acid sequence R-R-R-K-R-S-V-C-T (SEQ ID NO: 129) corresponds to FCS variant F82. In one example, the second polynucleotide encodes a first cleavage site comprising an amino acid sequence R-R-R-K-R-S-V-E-T (SEQ ID NO: 130). In one example, the cleavage site comprising an amino acid sequence R-R-R-K-R-S-V-E-T (SEQ ID NO: 130) corresponds to FCS variant F83. In one example, the second polynucleotide encodes a first cleavage site comprising an amino acid sequence R-R-R-K-R-S-V-Q-T (SEQ ID NO: 131). In one example, the cleavage site comprising an amino acid sequence R-R-R-K-R-S-V-Q-T (SEQ ID NO: 131) corresponds to FCS variant F84. In one example, the second polynucleotide encodes a first cleavage site comprising an amino acid sequence R-R-R-K-R-S-V-H-T (SEQ ID NO: 132). In one example, the cleavage site comprising an amino acid sequence R-R-R-K-R-S-V-H-T (SEQ ID NO: 132) corresponds to FCS variant F85. In one example, the second polynucleotide encodes a first cleavage site comprising an amino acid sequence R-R-R-K-R-S-V-I-T (SEQ ID NO: 133). In one example, the cleavage site comprising an amino acid sequence R-R-R-K-R-S-V-I-T (SEQ ID NO: 133) corresponds to FCS variant F86. In one example, the second polynucleotide encodes a first cleavage site comprising an amino acid sequence R-R-R-K-R-S-V-L-T (SEQ ID NO: 134). In one example, the cleavage site comprising an amino acid sequence R-R-R-K-R-S-V-L-T (SEQ ID NO: 134) corresponds to FCS variant F87. In one example, the second polynucleotide encodes a first cleavage site comprising an amino acid sequence R-R-R-K-R-S-V-M-T (SEQ ID NO: 135). In one example, the cleavage site comprising an amino acid sequence R-R-R-K-R-S-V-M-T (SEQ ID NO: 135) corresponds to FCS variant F88. In one example, the second polynucleotide encodes a first cleavage site comprising an amino acid sequence R-R-R-K-R-S-V-F-T (SEQ ID NO: 136). In one example, the cleavage site comprising an amino acid sequence R-R-R-K-R-S-V-F-T (SEQ ID NO: 136) corresponds to FCS variant F89. In one example, the second polynucleotide encodes a first cleavage site comprising an amino acid sequence R-R-R-K-R-S-V-S-T (SEQ ID NO: 137). In one example, the cleavage site comprising an amino acid sequence R-R-R-K-R-S-V-S-T (SEQ ID NO: 137) corresponds to FCS variant F90. In one example, the second polynucleotide encodes a first cleavage site comprising an amino acid sequence R-R-R-K-R-S-V-T-T (SEQ ID NO: 138). In one example, the cleavage site comprising an amino acid sequence R-R-R-K-R-S-V-T-T (SEQ ID NO: 138) corresponds to FCS variant F91. In one example, the second polynucleotide encodes a first cleavage site comprising an amino acid sequence R-R-R-K-R-S-V-W-T (SEQ ID NO: 139). In one example, the cleavage site comprising an amino acid sequence R-R-R-K-R-S-V-W-T (SEQ ID NO: 139) corresponds to FCS variant F92. In one example, the second polynucleotide encodes a first cleavage site comprising an amino acid sequence R-R-R-K-R-S-V-Y-T (SEQ ID NO: 140). In one example, the cleavage site comprising an amino acid sequence R-R-R-K-R-S-V-Y-T (SEQ ID NO: 140) corresponds to FCS variant F93. In one example, the second polynucleotide encodes a first cleavage site comprising an amino acid sequence R-R-R-K-R-S-V-V-T (SEQ ID NO: 141). In one example, the cleavage site comprising an amino acid sequence R-R-R-K-R-S-V-V-T (SEQ ID NO: 141) corresponds to FCS variant F94. In one example, the second polynucleotide encodes a first cleavage site comprising an amino acid sequence R-R-R-K-R-S-V-D-P (SEQ ID NO: 142). In one example, the cleavage site comprising an amino acid sequence R-R-R-K-R-S-V-D-P (SEQ ID NO: 142) corresponds to FCS variant F95. In one example, the second polynucleotide encodes a first cleavage site comprising an amino acid sequence R-R-R-K-R-S-V-D-G (SEQ ID NO: 143). In one example, the cleavage site comprising an amino acid sequence R-R-R-K-R-S-V-D-G (SEQ ID NO: 143) corresponds to FCS variant F96. In one example, the second polynucleotide encodes a first cleavage site comprising an amino acid sequence R-R-R-K-R-S-V-D-A (SEQ ID NO: 144). In one example, the cleavage site comprising an amino acid sequence R-R-R-K-R-S-V-D-A (SEQ ID NO: 144) corresponds to FCS variant F97. In one example, the second polynucleotide encodes a first cleavage site comprising an amino acid sequence R-R-R-K-R-S-V-D-R (SEQ ID NO: 145). In one example, the cleavage site comprising an amino acid sequence R-R-R-K-R-S-V-D-R (SEQ ID NO: 145) corresponds to FCS variant F98. In one example, the second polynucleotide encodes a first cleavage site comprising an amino acid sequence R-R-R-K-R-S-V-D-K (SEQ ID NO: 146). In one example, the cleavage site comprising an amino acid sequence R-R-R-K-R-S-V-D-K (SEQ ID NO: 146) corresponds to FCS variant F99. In one example, the second polynucleotide encodes a first cleavage site comprising an amino acid sequence R-R-R-K-R-S-V-D-N(SEQ ID NO: 147). In one example, the cleavage site comprising an amino acid sequence R-R-R-K-R-S-V-D-N(SEQ ID NO: 147) corresponds to FCS variant F100. In one example, the second polynucleotide encodes a first cleavage site comprising an amino acid sequence R-R-R-K-R-S-V-D-D (SEQ ID NO: 148). In one example, the cleavage site comprising an amino acid sequence R-R-R-K-R-S-V-D-D (SEQ ID NO: 148) corresponds to FCS variant F101. In one example, the second polynucleotide encodes a first cleavage site comprising an amino acid sequence R-R-R-K-R-S-V-D-C(SEQ ID NO: 149). In one example, the cleavage site comprising an amino acid sequence R-R-R-K-R-S-V-D-C(SEQ ID NO: 149) corresponds to FCS variant F102. In one example, the second polynucleotide encodes a first cleavage site comprising an amino acid sequence R-R-R-K-R-S-V-D-E (SEQ ID NO: 150). In one example, the cleavage site comprising an amino acid sequence R-R-R-K-R-S-V-D-E (SEQ ID NO: 150) corresponds to FCS variant F103. In one example, the second polynucleotide encodes a first cleavage site comprising an amino acid sequence R-R-R-K-R-S-V-D-Q (SEQ ID NO: 151). In one example, the cleavage site comprising an amino acid sequence R-R-R-K-R-S-V-D-Q (SEQ ID NO: 151) corresponds to FCS variant F104. In one example, the second polynucleotide encodes a first cleavage site comprising an amino acid sequence R-R-R-K-R-S-V-D-H (SEQ ID NO: 152). In one example, the cleavage site comprising an amino acid sequence R-R-R-K-R-S-V-D-H (SEQ ID NO: 152) corresponds to FCS variant F105. In one example, the second polynucleotide encodes a first cleavage site comprising an amino acid sequence R-R-R-K-R-S-V-D-I (SEQ ID NO: 153). In one example, the cleavage site comprising an amino acid sequence R-R-R-K-R-S-V-D-I (SEQ ID NO: 153) corresponds to FCS variant F106. In one example, the second polynucleotide encodes a first cleavage site comprising an amino acid sequence R-R-R-K-R-S-V-D-L (SEQ ID NO: 154). In one example, the cleavage site comprising an amino acid sequence R-R-R-K-R-S-V-D-L (SEQ ID NO: 154) corresponds to FCS variant F107. In one example, the second polynucleotide encodes a first cleavage site comprising an amino acid sequence R-R-R-K-R-S-V-D-M (SEQ ID NO: 155). In one example, the cleavage site comprising an amino acid sequence R-R-R-K-R-S-V-D-M (SEQ ID NO: 155) corresponds to FCS variant F108. In one example, the second polynucleotide encodes a first cleavage site comprising an amino acid sequence R-R-R-K-R-S-V-D-F (SEQ ID NO: 156). In one example, the cleavage site comprising an amino acid sequence R-R-R-K-R-S-V-D-F (SEQ ID NO: 156) corresponds to FCS variant F109. In one example, the second polynucleotide encodes a first cleavage site comprising an amino acid sequence R-R-R-K-R-S-V-D-S(SEQ ID NO: 157). In one example, the cleavage site comprising an amino acid sequence R-R-R-K-R-S-V-D-S(SEQ ID NO: 157) corresponds to FCS variant F110. In one example, the second polynucleotide encodes a first cleavage site comprising an amino acid sequence R-R-R-K-R-S-V-D-W (SEQ ID NO: 158). In one example, the cleavage site comprising an amino acid sequence R-R-R-K-R-S-V-D-W (SEQ ID NO: 158) corresponds to FCS variant F111. In one example, the second polynucleotide encodes a first cleavage site comprising an amino acid sequence R-R-R-K-R-S-V-D-Y (SEQ ID NO: 159). In one example, the cleavage site comprising an amino acid sequence R-R-R-K-R-S-V-D-Y (SEQ ID NO: 159) corresponds to FCS variant F112. In one example, the second polynucleotide encodes a first cleavage site comprising an amino acid sequence R-R-R-K-R-S-V-D-V (SEQ ID NO: 160). In one example, the cleavage site comprising an amino acid sequence R-R-R-K-R-S-V-D-V (SEQ ID NO: 160) corresponds to FCS variant F113.

In one example, the expression system for an antigen binding molecule described herein comprises a third polynucleotide. In one example, the third polynucleotide encodes a second cleavage site comprising a 2A polypeptide or a fragment thereof. In one example, the second cleavage site is a 2A polypeptide or a fragment thereof. The terms, not limited to “2A polypeptide”, “2A peptide”, “2A protein” as used herein, refer to a peptide that mediates “self-cleavage” or “self-processing” of proteins during translation in eukaryotic cells. The 2A polypeptide is, for example, usually 18-25 amino-acid in length, and originates from viruses. 2A polypeptide is capable of self-cleaving, which occurs co-translationally between, for example, between the last two amino acids, glycine and proline. In one example, the 2A polypeptide or a fragment thereof is selected from a group consisting of P2A, F2A, E2A and T2A, or a fragment thereof. F2A is a 2A peptide derived from foot-and-mouth disease virus; E2A is a 2A peptide derived from equine rhinitis A virus; T2A is a 2A peptide derived from Thosea asigna virus; P2A is a 2A peptide derived from porcine teschovirus-1. In another example, the 2A polypeptide or a fragment thereof is a P2A polypeptide or a fragment thereof. In one example, the second polypeptide encoding a first cleavage site requires linkage with 2A peptides to ensure efficient co-expression of antibody light chain and heavy chain genes. In another example, the 2A polypeptide or a fragment there of is a T2A polypeptide or a fragment thereof. In one example, the P2A polypeptide is ATNFSLLKQAGDVEENPGP (SEQ ID NO: 20). In another example, the F2A polypeptide is APVKQTLNFDLLKLAGDVESNPGP (SEQ ID NO: 21). In another example, the E2A polypeptide is QCTNYALLKLAGDVESNPGP (SEQ ID NO: 22). In another example, the T2A polypeptide is EGRGSLLTCGDVEENPGP (SEQ ID NO: 23). In one example, the cleavage of 2A polypeptides such as T2A (EGRGSLLTCGDVEENPGP; SEQ ID NO: 23) happens at the last two amino acids, G and P. In one example, the amino acid sequence EGRGSLLTCGDVEENPG (SEQ ID NO: 24) will be attached to the first part of the antigen binding molecule (such as the light chain of an antibody) if there is no Furin cleavage sequence or its variant in front of the 2A polypeptide. In one example, the second polynucleotide is located downstream of the first polynucleotide.

In one example, the expression system for an antigen binding molecule described herein comprises a fourth polynucleotide encoding a second part of the antigen binding molecule. In one example, the second part of the antigen binding molecule is a heavy chain of an antibody or other protein construct. In another example, the second part of the antigen binding molecule is the heavy chain of an antibody or other protein construct. In one example, the fourth polynucleotide is located downstream of the third polynucleotide. In one example, when the first and second cleavage sites are cleaved, an antigen binding molecule comprising the first and second parts of the antigen binding molecule is released.

In one aspect, the present disclosure refers to an expression system for an antigen binding molecule, wherein the antigen binding molecule is either secretable or membrane-bound, comprising:
a first polynucleotide encoding a first part of the antigen binding molecule;
a second polynucleotide encoding a first cleavage site comprising an amino acid sequence Z-R-X1-X2-R-X3-X4-X5-X6 (SEQ ID NO: 256), wherein the second polynucleotide is located downstream of the first polynucleotide;
a third polynucleotide encoding a second cleavage site comprising a 2A polypeptide or a fragment thereof, wherein the third polynucleotide is located downstream of the second polynucleotide;
a fourth polynucleotide encoding a second part of the antigen binding molecule, wherein the fourth polynucleotide is located downstream of the third polynucleotide;
a fifth polynucleotide encoding a third cleavage site comprising a Furin cleavage sequence variant comprising:
   (i) a Furin consensus sequence R-X-K-R (SEQ ID NO: 18) and at least the first five amino acids derived from a 2A polypeptide; or
   (ii) a Furin consensus sequence R-X-R-R (SEQ ID NO: 19) and at least the first five amino acids derived from a 2A polypeptide,
wherein the fifth polynucleotide is located downstream of the fourth polynucleotide; and
a sixth polynucleotide encoding a membrane anchor polypeptide, wherein the sixth polynucleotide is located downstream of the fifth polynucleotide;
wherein when the first, second and third cleavage sites are cleaved, a secretable antigen binding molecule comprising the first and second parts of the antigen binding molecule is released;
wherein when the first and second cleavage sites are cleaved and the third cleavage site is not cleaved, a membrane-bound antigen binding molecule comprising the first and second parts of the antigen binding molecule, the third cleavage site, and the membrane anchor polypeptide is released,
wherein Z is R, P, G, A, K, N, Q, H, I, L, M, F, S, T, W, Y, or V,
wherein X1, X2, X3, X4, X5 and X6 is R, P, G, A, D, C, E, K, N, Q, H, I, L, M, F, S, T, W, Y, or V, and
wherein X is any amino acid.

In one example of the preceding aspect, the present disclosure refers to an expression system for an antigen binding molecule, wherein the antigen binding molecule is either secretable or membrane-bound, comprising:
a first polynucleotide encoding a first part of the antigen binding molecule;
a second polynucleotide encoding a first cleavage site comprising an amino acid sequence R-R-X1-X2-R-X3-X4-X5-X6 (SEQ ID NO: 1), wherein the second polynucleotide is located downstream of the first polynucleotide;
a third polynucleotide encoding a second cleavage site comprising a 2A polypeptide or a fragment thereof, wherein the third polynucleotide is located downstream of the second polynucleotide;
a fourth polynucleotide encoding a second part of the antigen binding molecule, wherein the fourth polynucleotide is located downstream of the third polynucleotide;
a fifth polynucleotide encoding a third cleavage site comprising a Furin cleavage sequence variant comprising:
   (i) a Furin consensus sequence R-X-K-R (SEQ ID NO: 18) and at least the first five amino acids derived from a 2A polypeptide; or
   (ii) a Furin consensus sequence R-X-R-R (SEQ ID NO: 19) and at least the first five amino acids derived from a 2A polypeptide,
wherein the fifth polynucleotide is located downstream of the fourth polynucleotide; and
a sixth polynucleotide encoding a membrane anchor polypeptide, wherein the sixth polynucleotide is located downstream of the fifth polynucleotide;
wherein when the first, second and third cleavage sites are cleaved, a secretable antigen binding molecule comprising the first and second parts of the antigen binding molecule is released;
wherein when the first and second cleavage sites are cleaved and the third cleavage site is not cleaved, a membrane-bound antigen binding molecule comprising the first and second parts of the antigen binding molecule, the third cleavage site, and the membrane anchor polypeptide is released.

In one example, the antigen binding molecule produced or derived from the expression system as disclosed herein is capable of being secreted from a cell and may be known as a “secretable antigen binding molecule”. In one example, the term “secretable antigen binding molecule” used herein refers to an antigen binding molecule that is capable of being secreted from a cell, wherein the antigen binding molecule comprises first and second parts of the antigen binding molecule derived from the expression system disclosed herein.

In one example, the antigen binding molecule produced or derived from the expression system as disclosed herein is capable of being anchored or bound to a cell membrane and may be known as a “membrane bound antigen binding molecule”. In one example, the term “membrane bound antigen binding molecule” as used herein refers to an antigen binding molecule that is capable of being anchored or bound to a cell membrane, wherein the antigen binding molecule comprises first and second parts of the antigen binding molecule, a third cleavage site and a membrane anchor polypeptide derived from the expression system as disclosed herein. The levels of membrane bound antigen binding molecule is determined by the display level. The term “display level” used herein refers to the expression of a membrane-bound antigen binding molecule on a cell surface, wherein the membrane-bound antigen binding molecule comprises first and second parts of the antigen binding molecule.

In one example, the antigen binding molecule produced or derived from the expression system as disclosed herein may be an antigen binding molecule that remains within a cell, wherein it is not secreted from the cell or is trapped on the surface of the cell. In one example, the antigen binding molecule may remain within the cell if the signal peptides on them are removed. In one example, the antigen binding molecule may remain within the cell if the Furin consensus sequence or its variant between the second part of the antigen binding molecule (such as the heavy chain of an antibody) and the membrane anchor polypeptide (such as GPI) is removed. In one example, the antigen binding molecule may remain within the cell if the cleavage function of the Furin consensus sequence or its variant between the second part of the antigen binding molecule (such as the heavy chain of an antibody) and the membrane anchor polypeptide (such as GPI) is abolished. In one example, the Furin consensus sequence or its variant and the GPI affect the amount of antigen binding molecules that are trapped on the surface of the cell.

In one example, the expression system for an antigen binding molecule, wherein the antigen binding molecule is either secretable or membrane-bound, comprises a fifth polynucleotide encoding a third cleavage site comprising a Furin cleavage sequence variant. In one example, the third cleavage site comprises a Furin cleavage sequence variant comprising a Furin consensus sequence R-X-K-R (SEQ ID NO: 18) or R-X-R-R (SEQ ID NO: 19) and at least the first amino acid derived from a 2A polypeptide, at least the first two amino acids derived from a 2A polypeptide, at least the first three amino acids derived from a 2A polypeptide, at least the first four amino acids derived from a 2A polypeptide, or at least the first five amino acids derived from a 2A polypeptide. In one example, the third cleavage site comprises a Furin cleavage sequence variant comprising a Furin consensus sequence R-X-K-R (SEQ ID NO: 18) or R-X-R-R (SEQ ID NO: 19) and at least the first five amino acids derived from a 2A polypeptide.

In one example, the expression system for an antigen binding molecule, wherein the antigen binding molecule is either secretable or membrane-bound, comprises a sixth polynucleotide encoding a membrane anchor polypeptide. In one example, the sixth polynucleotide is located downstream of the fifth polynucleotide. In one example, when the first, second and third cleavage sites are cleaved, a secretable antigen binding molecule comprising the first and second parts of the antigen binding molecule is released. In one example, when the first and second cleavage sites are cleaved and the third cleavage site is not cleaved, a membrane-bound antigen binding molecule comprising the first and second parts of the antigen binding molecule, the third cleavage site, and the membrane anchor polypeptide is released. In one example, the fifth polynucleotide does not require linkage with 2A peptides for the simultaneous display and secretion of antibodies.

In one example, the expression system for an antigen binding molecule described herein comprises a second polynucleotide encoding a first cleavage site comprising an amino acid sequence Z-R-X1-X2-R-X3-X4-X5-X6 (SEQ ID NO: 256). In one example, Z is Arginine (R), Proline (P), Glycine (G), Alanine (A), Lysine (K), Asparagine (N), Glutamine (Q), Histidine (H), Isoleucine (I), Leucine (L), Methionine (M), Phenylalanine (F), Serine(S), Threonine (T), Tryptophan (W), Tyrosine (Y), or Valine (V); X3 is any one of Serine(S), Alanine (A), Aspartic acid (D), or Glutamic acid (E); X4 is any one of Valine (V), Alanine (A), Isoleucine (I), Leucine (L), or Threonine (T); X5 is any one of Arginine (R), Proline (P), Glycine (G), Alanine (A), Aspartic acid (D), Cysteine (C), Glutamic acid (E), Lysine (K), Asparagine (N), Glutamine (Q), Histidine (H), Isoleucine (I), Leucine (L), Methionine (M), Phenylalanine (F), Serine(S), Threonine (T), Tryptophan (W), Tyrosine (Y), or Valine (V); and X6 is any one of Arginine (R), Proline (P), Glycine (G), Alanine (A), Aspartic acid (D), Cysteine (C), Glutamic acid (E), Lysine (K), Asparagine (N), Glutamine (Q), Histidine (H), Isoleucine (I), Leucine (L), Methionine (M), Phenylalanine (F), Serine(S), Threonine (T), Tryptophan (W), Tyrosine (Y), or Valine (V). In one example, Z is Arginine (R). In one example, the expression system for an antigen binding molecule described herein comprises a second polynucleotide encoding a first cleavage site comprising an amino acid sequence R-R-X1-X2-R-X3-X4-X5-X6 (SEQ ID NO: 1). In one example, X1-X6 may be any amino acids. In one example, X1 is Arginine (R), X2 is Lysine (K) or Arginine (R), and X3, X4, X5, and X6 is any one of Proline (P), Glycine (G), Alanine (A), Aspartic Acid (D), Cysteine (C), Glutamic Acid (E), Lysine (K), Asparagine (N), Glutamine (Q), Histidine (H), Isoleucine (I), Leucine (L), Methionine (M), Phenylalanine (F), Serine(S), Threonine (T), Tryptophan (W), Tyrosine (Y), or Valine (V). In one example, X1 is Arginine (R), X2 is Lysine (K) or Arginine (R), X3 is Serine(S) or Alanine (A), X4 is Valine (V) or Leucine (L), X5 is Aspartic Acid (D) or Serine(S), and X6 is Threonine (T) or Leucine (L). In one example, X3 is any one of Proline (P), Glycine (G), Alanine (A), Aspartic Acid (D), Glutamic Acid (E), Histidine (H), Isoleucine (I), Leucine (L), Methionine (M), Phenylalanine (F), or Serine (S); X4 is any one of Proline (P), Alanine (A), Isoleucine (I), Leucine (L), Threonine (T), or Valine (V); X5 is any one of Arginine (R), Proline (P), Glycine (G), Alanine (A), Aspartic acid (D), Cysteine (C), Glutamic acid (E), Lysine (K), Asparagine (N), Glutamine (Q), Histidine (H), Isoleucine (I), Leucine (L), Methionine (M), Phenylalanine (F), Serine(S), Threonine (T), Tryptophan (W), Tyrosine (Y), or Valine (V); and X6 is any one of Arginine (R), Proline (P), Glycine (G), Alanine (A), Aspartic acid (D), Cysteine (C), Glutamic acid (E), Lysine (K), Asparagine (N), Glutamine (Q), Histidine (H), Isoleucine (I), Leucine (L), Methionine (M), Phenylalanine (F), Serine(S), Threonine (T), Tryptophan (W), Tyrosine (Y), or Valine (V). In one example, Z is any one of Arginine (R), Proline (P), Glycine (G), Alanine (A), Lysine (K), Asparagine (N), Glutamine (Q), Histidine (H), Isoleucine (I), Leucine (L), Methionine (M), Phenylalanine (F), Serine(S), Threonine (T), Tryptophan (W), Tyrosine (Y), or Valine (V); X3 is any one of Serine(S), Alanine (A), Aspartic Acid (D), or Glutamic Acid (E); X4 is any one of Valine (V), Alanine (A), Isoleucine (I), Leucine (L), or Threonine (T); X5 is any one of Arginine (R), Proline (P), Glycine (G), Alanine (A), Aspartic Acid (D), Cysteine (C), Glutamic Acid (E), Lysine (K), Asparagine (N), Glutamine (Q), Histidine (H), Isoleucine (I), Leucine (L), Methionine (M), Phenylalanine (F), Serine(S), Threonine (T), Tryptophan (W), Tyrosine (Y), or Valine (V); and X6 is any one of Arginine (R), Proline (P), Glycine (G), Alanine (A), Aspartic Acid (D), Cysteine (C), Glutamic Acid (E), Lysine (K), Asparagine (N), Glutamine (Q), Histidine (H), Isoleucine (I), Leucine (L), Methionine (M), Phenylalanine (F), Serine(S), Threonine (T), Tryptophan (W), Tyrosine (Y), or Valine (V).

In one example, the first cleavage site encoded by the second polynucleotide comprises an amino acid sequence set forth in SEQ ID NOs: 2-17 and SEQ ID NOs: 66-160. In one example, the first cleavage site encoded by the second polynucleotide comprises an amino acid sequence set forth in SEQ ID NO: 2. In one example, the first cleavage site encoded by the second polynucleotide comprises an amino acid sequence set forth in SEQ ID NO: 3. In one example, the first cleavage site encoded by the second polynucleotide comprises an amino acid sequence set forth in SEQ ID NO: 4. In one example, the first cleavage site encoded by the second polynucleotide comprises an amino acid sequence set forth in SEQ ID NO: 5. In one example, the first cleavage site encoded by the second polynucleotide comprises an amino acid sequence set forth in SEQ ID NO: 6. In one example, the first cleavage site encoded by the second polynucleotide comprises an amino acid sequence set forth in SEQ ID NO: 7. In one example, the first cleavage site encoded by the second polynucleotide comprises an amino acid sequence set forth in SEQ ID NO: 8. In one example, the first cleavage site encoded by the second polynucleotide comprises an amino acid sequence set forth in SEQ ID NO: 9. In one example, the first cleavage site encoded by the second polynucleotide comprises an amino acid sequence set forth in SEQ ID NO: 10. In one example, the first cleavage site encoded by the second polynucleotide comprises an amino acid sequence set forth in SEQ ID NO: 11. In one example, the first cleavage site encoded by the second polynucleotide comprises an amino acid sequence set forth in SEQ ID NO: 12. In one example, the first cleavage site encoded by the second polynucleotide comprises an amino acid sequence set forth in SEQ ID NO: 13. In one example, the first cleavage site encoded by the second polynucleotide comprises an amino acid sequence set forth in SEQ ID NO: 14. In one example, the first cleavage site encoded by the second polynucleotide comprises an amino acid sequence set forth in SEQ ID NO: 15. In one example, the first cleavage site encoded by the second polynucleotide comprises an amino acid sequence set forth in SEQ ID NO: 16. In one example, the first cleavage site encoded by the second polynucleotide comprises an amino acid sequence set forth in SEQ ID NO: 17. In one example, the first cleavage site encoded by the second polynucleotide comprises an amino acid sequence set forth in SEQ ID NO: 66. In one example, the first cleavage site encoded by the second polynucleotide comprises an amino acid sequence set forth in SEQ ID NO: 67. In one example, the first cleavage site encoded by the second polynucleotide comprises an amino acid sequence set forth in SEQ ID NO: 68. In one example, the first cleavage site encoded by the second polynucleotide comprises an amino acid sequence set forth in SEQ ID NO: 69. In one example, the first cleavage site encoded by the second polynucleotide comprises an amino acid sequence set forth in SEQ ID NO: 70. In one example, the first cleavage site encoded by the second polynucleotide comprises an amino acid sequence set forth in SEQ ID NO: 71. In one example, the first cleavage site encoded by the second polynucleotide comprises an amino acid sequence set forth in SEQ ID NO: 72. In one example, the first cleavage site encoded by the second polynucleotide comprises an amino acid sequence set forth in SEQ ID NO: 73. In one example, the first cleavage site encoded by the second polynucleotide comprises an amino acid sequence set forth in SEQ ID NO: 74. In one example, the first cleavage site encoded by the second polynucleotide comprises an amino acid sequence set forth in SEQ ID NO: 75. In one example, the first cleavage site encoded by the second polynucleotide comprises an amino acid sequence set forth in SEQ ID NO: 76. In one example, the first cleavage site encoded by the second polynucleotide comprises an amino acid sequence set forth in SEQ ID NO: 77. In one example, the first cleavage site encoded by the second polynucleotide comprises an amino acid sequence set forth in SEQ ID NO: 78. In one example, the first cleavage site encoded by the second polynucleotide comprises an amino acid sequence set forth in SEQ ID NO: 79. In one example, the first cleavage site encoded by the second polynucleotide comprises an amino acid sequence set forth in SEQ ID NO: 80. In one example, the first cleavage site encoded by the second polynucleotide comprises an amino acid sequence set forth in SEQ ID NO: 81. In one example, the first cleavage site encoded by the second polynucleotide comprises an amino acid sequence set forth in SEQ ID NO: 82. In one example, the first cleavage site encoded by the second polynucleotide comprises an amino acid sequence set forth in SEQ ID NO: 83. In one example, the first cleavage site encoded by the second polynucleotide comprises an amino acid sequence set forth in SEQ ID NO: 84. In one example, the first cleavage site encoded by the second polynucleotide comprises an amino acid sequence set forth in SEQ ID NO: 85. In one example, the first cleavage site encoded by the second polynucleotide comprises an amino acid sequence set forth in SEQ ID NO: 86. In one example, the first cleavage site encoded by the second polynucleotide comprises an amino acid sequence set forth in SEQ ID NO: 87. In one example, the first cleavage site encoded by the second polynucleotide comprises an amino acid sequence set forth in SEQ ID NO: 88. In one example, the first cleavage site encoded by the second polynucleotide comprises an amino acid sequence set forth in SEQ ID NO: 89. In one example, the first cleavage site encoded by the second polynucleotide comprises an amino acid sequence set forth in SEQ ID NO: 90. In one example, the first cleavage site encoded by the second polynucleotide comprises an amino acid sequence set forth in SEQ ID NO: 91. In one example, the first cleavage site encoded by the second polynucleotide comprises an amino acid sequence set forth in SEQ ID NO: 92. In one example, the first cleavage site encoded by the second polynucleotide comprises an amino acid sequence set forth in SEQ ID NO: 93. In one example, the first cleavage site encoded by the second polynucleotide comprises an amino acid sequence set forth in SEQ ID NO: 94. In one example, the first cleavage site encoded by the second polynucleotide comprises an amino acid sequence set forth in SEQ ID NO: 95. In one example, the first cleavage site encoded by the second polynucleotide comprises an amino acid sequence set forth in SEQ ID NO: 96. In one example, the first cleavage site encoded by the second polynucleotide comprises an amino acid sequence set forth in SEQ ID NO: 97. In one example, the first cleavage site encoded by the second polynucleotide comprises an amino acid sequence set forth in SEQ ID NO: 98. In one example, the first cleavage site encoded by the second polynucleotide comprises an amino acid sequence set forth in SEQ ID NO: 99. In one example, the first cleavage site encoded by the second polynucleotide comprises an amino acid sequence set forth in SEQ ID NO: 100. In one example, the first cleavage site encoded by the second polynucleotide comprises an amino acid sequence set forth in SEQ ID NO: 101. In one example, the first cleavage site encoded by the second polynucleotide comprises an amino acid sequence set forth in SEQ ID NO: 102. In one example, the first cleavage site encoded by the second polynucleotide comprises an amino acid sequence set forth in SEQ ID NO: 103. In one example, the first cleavage site encoded by the second polynucleotide comprises an amino acid sequence set forth in SEQ ID NO: 104. In one example, the first cleavage site encoded by the second polynucleotide comprises an amino acid sequence set forth in SEQ ID NO: 105. In one example, the first cleavage site encoded by the second polynucleotide comprises an amino acid sequence set forth in SEQ ID NO: 106. In one example, the first cleavage site encoded by the second polynucleotide comprises an amino acid sequence set forth in SEQ ID NO: 107. In one example, the first cleavage site encoded by the second polynucleotide comprises an amino acid sequence set forth in SEQ ID NO: 108. In one example, the first cleavage site encoded by the second polynucleotide comprises an amino acid sequence set forth in SEQ ID NO: 109. In one example, the first cleavage site encoded by the second polynucleotide comprises an amino acid sequence set forth in SEQ ID NO: 110. In one example, the first cleavage site encoded by the second polynucleotide comprises an amino acid sequence set forth in SEQ ID NO: 111. In one example, the first cleavage site encoded by the second polynucleotide comprises an amino acid sequence set forth in SEQ ID NO: 112. In one example, the first cleavage site encoded by the second polynucleotide comprises an amino acid sequence set forth in SEQ ID NO: 113. In one example, the first cleavage site encoded by the second polynucleotide comprises an amino acid sequence set forth in SEQ ID NO: 114. In one example, the first cleavage site encoded by the second polynucleotide comprises an amino acid sequence set forth in SEQ ID NO: 115. In one example, the first cleavage site encoded by the second polynucleotide comprises an amino acid sequence set forth in SEQ ID NO: 116. In one example, the first cleavage site encoded by the second polynucleotide comprises an amino acid sequence set forth in SEQ ID NO: 117. In one example, the first cleavage site encoded by the second polynucleotide comprises an amino acid sequence set forth in SEQ ID NO: 118. In one example, the first cleavage site encoded by the second polynucleotide comprises an amino acid sequence set forth in SEQ ID NO: 119. In one example, the first cleavage site encoded by the second polynucleotide comprises an amino acid sequence set forth in SEQ ID NO: 120. In one example, the first cleavage site encoded by the second polynucleotide comprises an amino acid sequence set forth in SEQ ID NO: 121. In one example, the first cleavage site encoded by the second polynucleotide comprises an amino acid sequence set forth in SEQ ID NO: 122. In one example, the first cleavage site encoded by the second polynucleotide comprises an amino acid sequence set forth in SEQ ID NO: 123. In one example, the first cleavage site encoded by the second polynucleotide comprises an amino acid sequence set forth in SEQ ID NO: 124. In one example, the first cleavage site encoded by the second polynucleotide comprises an amino acid sequence set forth in SEQ ID NO: 125. In one example, the first cleavage site encoded by the second polynucleotide comprises an amino acid sequence set forth in SEQ ID NO: 126. In one example, the first cleavage site encoded by the second polynucleotide comprises an amino acid sequence set forth in SEQ ID NO: 127. In one example, the first cleavage site encoded by the second polynucleotide comprises an amino acid sequence set forth in SEQ ID NO: 128. In one example, the first cleavage site encoded by the second polynucleotide comprises an amino acid sequence set forth in SEQ ID NO: 129. In one example, the first cleavage site encoded by the second polynucleotide comprises an amino acid sequence set forth in SEQ ID NO: 130. In one example, the first cleavage site encoded by the second polynucleotide comprises an amino acid sequence set forth in SEQ ID NO: 131. In one example, the first cleavage site encoded by the second polynucleotide comprises an amino acid sequence set forth in SEQ ID NO: 132. In one example, the first cleavage site encoded by the second polynucleotide comprises an amino acid sequence set forth in SEQ ID NO: 133. In one example, the first cleavage site encoded by the second polynucleotide comprises an amino acid sequence set forth in SEQ ID NO: 134. In one example, the first cleavage site encoded by the second polynucleotide comprises an amino acid sequence set forth in SEQ ID NO: 135. In one example, the first cleavage site encoded by the second polynucleotide comprises an amino acid sequence set forth in SEQ ID NO: 136. In one example, the first cleavage site encoded by the second polynucleotide comprises an amino acid sequence set forth in SEQ ID NO: 137. In one example, the first cleavage site encoded by the second polynucleotide comprises an amino acid sequence set forth in SEQ ID NO: 138. In one example, the first cleavage site encoded by the second polynucleotide comprises an amino acid sequence set forth in SEQ ID NO: 139. In one example, the first cleavage site encoded by the second polynucleotide comprises an amino acid sequence set forth in SEQ ID NO: 140. In one example, the first cleavage site encoded by the second polynucleotide comprises an amino acid sequence set forth in SEQ ID NO: 141. In one example, the first cleavage site encoded by the second polynucleotide comprises an amino acid sequence set forth in SEQ ID NO: 142. In one example, the first cleavage site encoded by the second polynucleotide comprises an amino acid sequence set forth in SEQ ID NO: 143. In one example, the first cleavage site encoded by the second polynucleotide comprises an amino acid sequence set forth in SEQ ID NO: 144. In one example, the first cleavage site encoded by the second polynucleotide comprises an amino acid sequence set forth in SEQ ID NO: 145. In one example, the first cleavage site encoded by the second polynucleotide comprises an amino acid sequence set forth in SEQ ID NO: 146. In one example, the first cleavage site encoded by the second polynucleotide comprises an amino acid sequence set forth in SEQ ID NO: 147. In one example, the first cleavage site encoded by the second polynucleotide comprises an amino acid sequence set forth in SEQ ID NO: 148. In one example, the first cleavage site encoded by the second polynucleotide comprises an amino acid sequence set forth in SEQ ID NO: 149. In one example, the first cleavage site encoded by the second polynucleotide comprises an amino acid sequence set forth in SEQ ID NO: 150. In one example, the first cleavage site encoded by the second polynucleotide comprises an amino acid sequence set forth in SEQ ID NO: 151. In one example, the first cleavage site encoded by the second polynucleotide comprises an amino acid sequence set forth in SEQ ID NO: 152. In one example, the first cleavage site encoded by the second polynucleotide comprises an amino acid sequence set forth in SEQ ID NO: 153. In one example, the first cleavage site encoded by the second polynucleotide comprises an amino acid sequence set forth in SEQ ID NO: 154. In one example, the first cleavage site encoded by the second polynucleotide comprises an amino acid sequence set forth in SEQ ID NO: 155. In one example, the first cleavage site encoded by the second polynucleotide comprises an amino acid sequence set forth in SEQ ID NO: 156. In one example, the first cleavage site encoded by the second polynucleotide comprises an amino acid sequence set forth in SEQ ID NO: 157. In one example, the first cleavage site encoded by the second polynucleotide comprises an amino acid sequence set forth in SEQ ID NO: 158. In one example, the first cleavage site encoded by the second polynucleotide comprises an amino acid sequence set forth in SEQ ID NO: 159. In one example, the first cleavage site encoded by the second polynucleotide comprises an amino acid sequence set forth in SEQ ID NO: 160. In one example, the first cleavage site encoded by the second polynucleotide comprises an amino acid sequence set forth in SEQ ID NOs: 2-17, 66-70, 74-84, 86, 87, 91, 93, 95-99, 104, 106, 115, 116, 120, 123-129, and 130-160. In one example, the first cleavage site encoded by the second polynucleotide comprises an amino acid sequence set forth in 2, 87, 91, 93, 106, 115, 116, and 120.

In one example, the expression system for an antigen binding molecule described herein further comprises a polynucleotide encoding a linker Serine-Glycine-Serine-Glycine (SGSG) between the second and third polynucleotides.

In one example, the third cleavage site of the fifth polynucleotide of the expression system described herein comprises a Furin cleavage sequence variant comprising a Furin consensus sequence R-X-K-R (SEQ ID NO: 18). In one example, the third cleavage site of the fifth polynucleotide of the expression system described herein comprises a Furin cleavage sequence variant comprising Furin consensus sequence R-X-R-R (SEQ ID NO: 19). In one example, the third cleavage site of the fifth polynucleotide of the expression system described herein comprises a Furin cleavage sequence variant comprising a Furin consensus sequence R-X-K-R (SEQ ID NO: 18) and at least the first five amino acids derived from a 2A polypeptide. In one example, the third cleavage site of the fifth polynucleotide of the expression system described herein comprises a Furin cleavage sequence variant comprising Furin consensus sequence R-X-R-R (SEQ ID NO: 19) and at least the first five amino acids derived from a 2A polypeptide. In one example, the third cleavage site of the fifth polynucleotide of the expression system described herein comprises a Furin cleavage sequence variant comprising a Furin consensus sequence R-X-K-R (SEQ ID NO: 18) and the first five amino acids derived from a 2A polypeptide. In one example, the third cleavage site of the fifth polynucleotide of the expression system described herein comprises a Furin cleavage sequence variant comprising Furin consensus sequence R-X-R-R (SEQ ID NO: 19) and the first five amino acids derived from a 2A polypeptide. In one example, the first five amino acids derived from the 2A polypeptide comprise one or more point mutations. In one example, the third cleavage site comprises a Furin cleavage sequence variant comprising a Furin consensus sequence R-X-K-R (SEQ ID NO: 18) or R-X-R-R (SEQ ID NO: 19) and the first five amino acids derived from a 2A polypeptide, wherein the first five amino acids derived from the 2A polypeptide comprise one or more point mutations. In one example, the first five amino acids derived from the 2A polypeptide comprise one point mutation. In one example, the first five amino acids derived from the 2A polypeptide comprise two point mutations. In one example, the first five amino acids derived from the 2A polypeptide comprise three point mutations. In one example, the first five amino acids derived from the 2A polypeptide comprise four point mutations. In one example, the first five amino acids derived from the 2A polypeptide comprise five point mutations. In one example, the first five amino acids is derived from a 2A polypeptide selected from the group consisting of P2A, F2A, E2A and T2A. In one example, the first five amino acid is derived from P2A. In one example, the first five amino acid is derived from T2A. In one example, the first five amino acid is derived from P2A and the one or more point mutations is selected from the group consisting of A1P, A1G, T2G, T2P, N3P, N3A, F4P, F4A and S5P, as shown in Table 1 below. In one example, the FCS variants listed in Table 1 may be utilized for simultaneous display and secretion of antibodies, positioned between the antibody heavy chain and the membrane anchor. In one example, the fifth polynucleotide is located downstream of the fourth polynucleotide. It would also be understood that the 2A polypeptide fragment thereof refers to a section of the 2A polypeptide as disclosed herein. In one example, the 2A polypeptide fragment thereof comprises a section of at least 3 amino acids from the 2A polypeptide. In another example, the 2A polypeptide fragment thereof comprises a section of, but is not limited to, about 3-10 amino acids, or about 3, 4, 5, 6, 7, 8, 9, or 10 amino acids from the 2A polypeptide. In another example, the 2A polypeptide fragment thereof is selected from a group consisting of P2A, F2A, E2A and T2A fragment thereof. In another example, the 2A polypeptide fragment thereof is a P2A polypeptide fragment thereof. In another example, the 2A polypeptide fragment thereof is a T2A polypeptide fragment thereof. In one example, the 2A polypeptide fragment thereof can be the first 3-10 amino acids of the 2A polypeptide as disclosed herein, or the last 3-10 amino acids of the 2A polypeptide as disclosed herein. In another example, the 2A polypeptide fragment thereof is the first five amino acids of a P2A, F2A, E2A or T2A polypeptide. In another example, the P2A polypeptide fragment thereof is the first five amino acids of a P2A polypeptide. In another example, the T2A polypeptide fragment thereof is the first five amino acids of a T2A polypeptide. In one example, the term “point mutation” refers to a mutation where a single amino acid is substituted, inserted or deleted from an amino acid sequence.

TABLE 1

| Name of sequence | Sequence |
|---|---|
| First 5 amino acids | ATNFS (SEQ ID NO: 25) |
| of P2A | |
| A1P mutant | PTNFS (SEQ ID NO: 26) |
| A1G mutant | GTNFS (SEQ ID NO: 27) |
| T2G mutant | AGNFS (SEQ ID NO: 28) |
| T2P mutant | APNFS (SEQ ID NO: 29) |
| N3P mutant | ATPFS (SEQ ID NO: 30) |
| N3A mutant | ATAFS (SEQ ID NO: 31) |
| F4P mutant | ATNPS (SEQ ID NO: 32) |
| F4A mutant | ATNAS (SEQ ID NO: 33) |
| S5P mutant | ATNFP (SEQ ID NO: 34) |

In one example, the membrane anchor polypeptide encoded by the sixth polynucleotide comprises glycophospholipid transmembrane domain (GPI), platelet-derived growth factor receptor (PDGFR) beta chain transmembrane domain (PTM), or immunoglobulin C2-type extracellular-transmembrane-cytosolic domains of murin B7-1 antigen. In another example, the membrane anchor polypeptide is glycophospholipid transmembrane domain (GPI). The terms not limiting to “glycophospholipid transmembrane domain”, “glycophospholipid membrane anchor”, “GM” or “GPI” used herein refer to membrane proteins that are anchored to a membrane in a cell by a structure comprising phosphatidylinositol, carbohydrate, and ethanolamine. In one example, the glycophospholipid transmembrane domain (GPI) is a glycosidylphosphatidylinositol membrane anchor derived from human decay-accelerating factor.

In one example, the second polynucleotide of the expression system as disclosed herein encodes a first cleavage site having a high or improved cleavage efficiency. In one example, the second polynucleotide encodes a first cleavage site having a higher cleavage efficiency of at least 80%, 81%, 82%, 83%, 84%, 85%, 86%, 87%, 88%, 89%, 90%, 91%, 92%, 93%, 94%, 95%, 96%, 97%, 98% or 99% compared to an expression system comprising a second polynucleotide that encodes a first cleavage site comprising a Furin consensus sequence R-X-K-R (SEQ ID NO: 18) or R-X-R-R (SEQ ID NO: 19).

In one example, the expression system for an antigen binding molecule described herein is capable of improving the accuracy of producing or releasing an antigen binding molecule comprising a first part of the antigen binding molecule having the correct polypeptide sequence. In one example, the expression system as disclosed herein is capable of releasing an antigen binding molecule comprising a first part of the antigen binding molecule having the correct polypeptide sequence, wherein the first part of the antigen binding molecule does not contain one or more residual amino acids from the second cleavage site after the cleavage of the first and second cleavage sites.

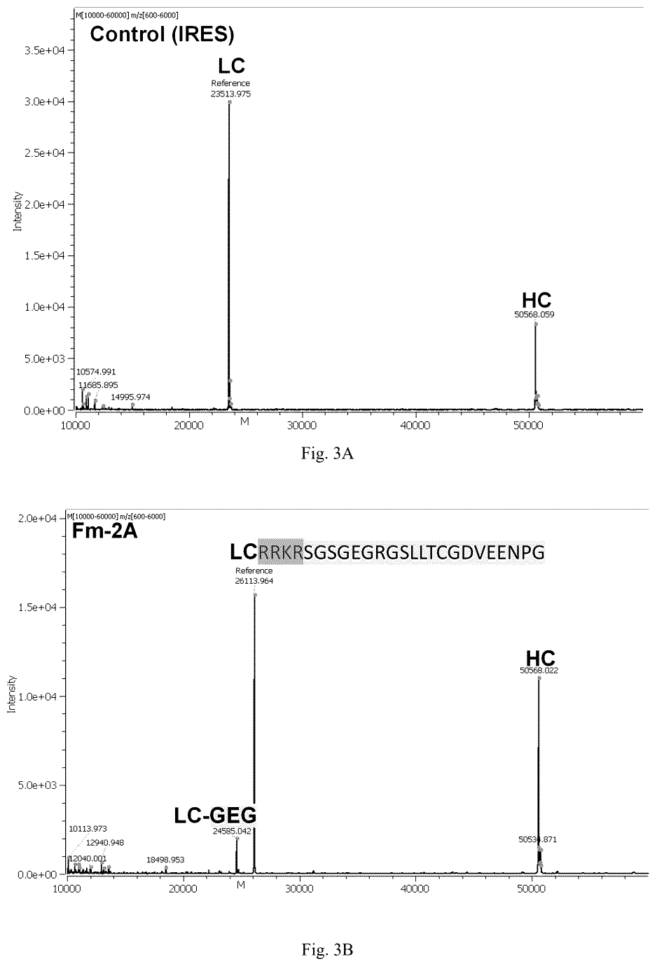

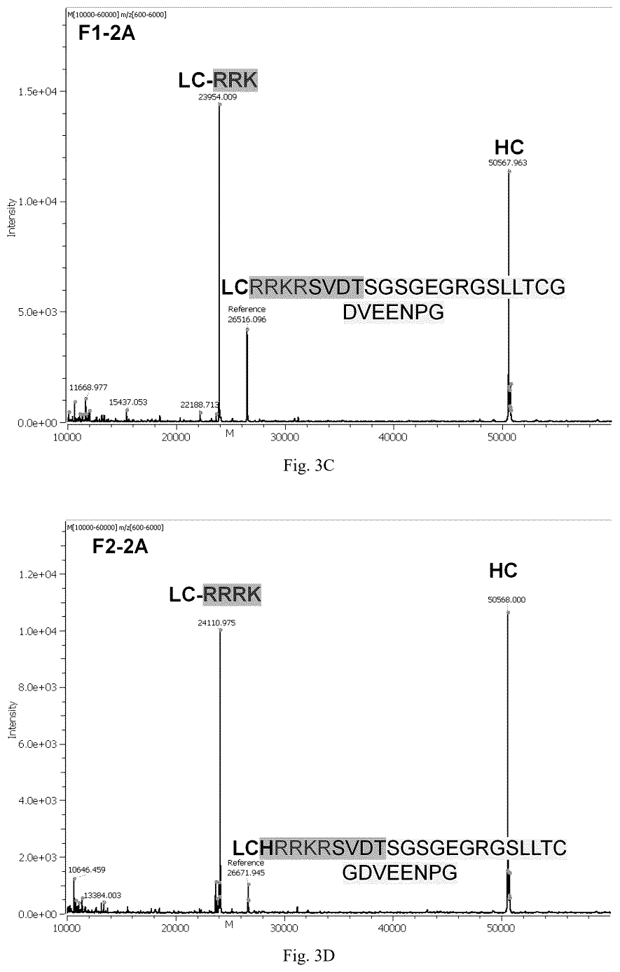

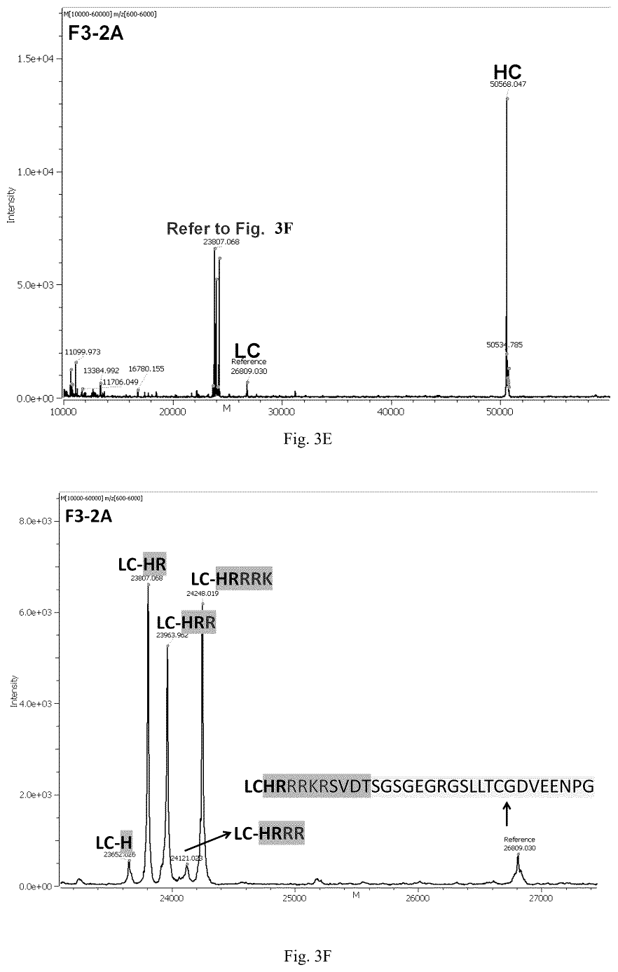

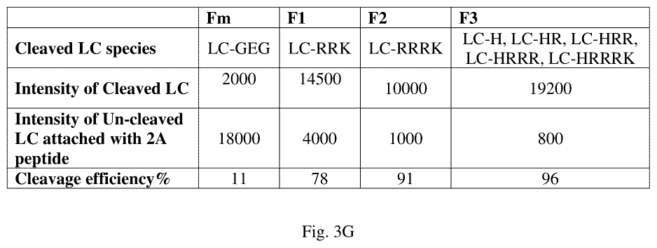

In one example, the expression system for an antigen binding molecule described herein is capable of improving the homogeneity of the antigen binding molecules produced. In one example, the term “homogeneity” means cleavage at the first cleavage site results in one major species of the first part of the antigen binding molecule (such as one major species of the light chain of an antibody as shown in FIG. 3D and FIG. 3G without attachment of 2A peptide) instead of multiple species of the first part of the antigen binding molecule (such as multiple species of the light chain of an antibody as shown in FIG. 3E, FIG. 3F and FIG. 3G). In one example, the expression system as disclosed herein is capable of improving the homogeneity of the antigen binding molecules produced, wherein homogeneity is achieved when a plurality of antigen binding molecules are produced, wherein each antigen binding molecule in the plurality of antigen binding molecules comprises a first part having the same amino acid sequence and/or molecular weight.

In one example, the expression system for an antigen binding molecule described herein is delivered to a target cell prior to transcription. In one example, the target cell is an animal cell, a yeast cell, a plant cell, an insect cell, or a fungal cell. In one example, the target cell is an animal cell. In one example, the animal cell is a mammalian cell.

In one example, the expression system for an antigen binding molecule described herein comprises a second polynucleotide comprising a nucleotide sequence set forth in SEQ ID NOs: 35-50 and SEQ ID NOs: 161-255. In one example, the expression system for an antigen-binding molecule described herein comprises a second polynucleotide with a nucleotide sequence set forth in SEQ ID NO: 35. In one example, the expression system for an antigen-binding molecule described herein comprises a second polynucleotide with a nucleotide sequence set forth in SEQ ID NO: 36. In one example, the expression system for an antigen-binding molecule described herein comprises a second polynucleotide with a nucleotide sequence set forth in SEQ ID NO: 37. In one example, the expression system for an antigen-binding molecule described herein comprises a second polynucleotide with a nucleotide sequence set forth in SEQ ID NO: 38. In one example, the expression system for an antigen-binding molecule described herein comprises a second polynucleotide with a nucleotide sequence set forth in SEQ ID NO: 39. In one example, the expression system for an antigen-binding molecule described herein comprises a second polynucleotide with a nucleotide sequence set forth in SEQ ID NO: 40. In one example, the expression system for an antigen-binding molecule described herein comprises a second polynucleotide with a nucleotide sequence set forth in SEQ ID NO: 41. In one example, the expression system for an antigen-binding molecule described herein comprises a second polynucleotide with a nucleotide sequence set forth in SEQ ID NO: 42. In one example, the expression system for an antigen-binding molecule described herein comprises a second polynucleotide with a nucleotide sequence set forth in SEQ ID NO: 43. In one example, the expression system for an antigen-binding molecule described herein comprises a second polynucleotide with a nucleotide sequence set forth in SEQ ID NO: 44. In one example, the expression system for an antigen-binding molecule described herein comprises a second polynucleotide with a nucleotide sequence set forth in SEQ ID NO: 45. In one example, the expression system for an antigen-binding molecule described herein comprises a second polynucleotide with a nucleotide sequence set forth in SEQ ID NO: 46. In one example, the expression system for an antigen-binding molecule described herein comprises a second polynucleotide with a nucleotide sequence set forth in SEQ ID NO: 47. In one example, the expression system for an antigen-binding molecule described herein comprises a second polynucleotide with a nucleotide sequence set forth in SEQ ID NO: 48. In one example, the expression system for an antigen-binding molecule described herein comprises a second polynucleotide with a nucleotide sequence set forth in SEQ ID NO: 49. In one example, the expression system for an antigen-binding molecule described herein comprises a second polynucleotide with a nucleotide sequence set forth in SEQ ID NO: 50. In one example, the expression system for an antigen-binding molecule described herein comprises a second polynucleotide with a nucleotide sequence set forth in SEQ ID NO: 161. In one example, the expression system for an antigen-binding molecule described herein comprises a second polynucleotide with a nucleotide sequence set forth in SEQ ID NO: 162. In one example, the expression system for an antigen-binding molecule described herein comprises a second polynucleotide with a nucleotide sequence set forth in SEQ ID NO: 163. In one example, the expression system for an antigen-binding molecule described herein comprises a second polynucleotide with a nucleotide sequence set forth in SEQ ID NO: 164. In one example, the expression system for an antigen-binding molecule described herein comprises a second polynucleotide with a nucleotide sequence set forth in SEQ ID NO: 165. In one example, the expression system for an antigen-binding molecule described herein comprises a second polynucleotide with a nucleotide sequence set forth in SEQ ID NO: 166. In one example, the expression system for an antigen-binding molecule described herein comprises a second polynucleotide with a nucleotide sequence set forth in SEQ ID NO: 167. In one example, the expression system for an antigen-binding molecule described herein comprises a second polynucleotide with a nucleotide sequence set forth in SEQ ID NO: 168. In one example, the expression system for an antigen-binding molecule described herein comprises a second polynucleotide with a nucleotide sequence set forth in SEQ ID NO: 169. In one example, the expression system for an antigen-binding molecule described herein comprises a second polynucleotide with a nucleotide sequence set forth in SEQ ID NO: 170. In one example, the expression system for an antigen-binding molecule described herein comprises a second polynucleotide with a nucleotide sequence set forth in SEQ ID NO: 171. In one example, the expression system for an antigen-binding molecule described herein comprises a second polynucleotide with a nucleotide sequence set forth in SEQ ID NO: 172. In one example, the expression system for an antigen-binding molecule described herein comprises a second polynucleotide with a nucleotide sequence set forth in SEQ ID NO: 173. In one example, the expression system for an antigen-binding molecule described herein comprises a second polynucleotide with a nucleotide sequence set forth in SEQ ID NO: 174. In one example, the expression system for an antigen-binding molecule described herein comprises a second polynucleotide with a nucleotide sequence set forth in SEQ ID NO: 175. In one example, the expression system for an antigen-binding molecule described herein comprises a second polynucleotide with a nucleotide sequence set forth in SEQ ID NO: 176. In one example, the expression system for an antigen-binding molecule described herein comprises a second polynucleotide with a nucleotide sequence set forth in SEQ ID NO: 177. In one example, the expression system for an antigen-binding molecule described herein comprises a second polynucleotide with a nucleotide sequence set forth in SEQ ID NO: 178. In one example, the expression system for an antigen-binding molecule described herein comprises a second polynucleotide with a nucleotide sequence set forth in SEQ ID NO: 179. In one example, the expression system for an antigen-binding molecule described herein comprises a second polynucleotide with a nucleotide sequence set forth in SEQ ID NO: 180. In one example, the expression system for an antigen-binding molecule described herein comprises a second polynucleotide with a nucleotide sequence set forth in SEQ ID NO: 181. In one example, the expression system for an antigen-binding molecule described herein comprises a second polynucleotide with a nucleotide sequence set forth in SEQ ID NO: 182. In one example, the expression system for an antigen-binding molecule described herein comprises a second polynucleotide with a nucleotide sequence set forth in SEQ ID NO: 183. In one example, the expression system for an antigen-binding molecule described herein comprises a second polynucleotide with a nucleotide sequence set forth in SEQ ID NO: 184. In one example, the expression system for an antigen-binding molecule described herein comprises a second polynucleotide with a nucleotide sequence set forth in SEQ ID NO: 185. In one example, the expression system for an antigen-binding molecule described herein comprises a second polynucleotide with a nucleotide sequence set forth in SEQ ID NO: 186. In one example, the expression system for an antigen-binding molecule described herein comprises a second polynucleotide with a nucleotide sequence set forth in SEQ ID NO: 187. In one example, the expression system for an antigen-binding molecule described herein comprises a second polynucleotide with a nucleotide sequence set forth in SEQ ID NO: 188. In one example, the expression system for an antigen-binding molecule described herein comprises a second polynucleotide with a nucleotide sequence set forth in SEQ ID NO: 189. In one example, the expression system for an antigen-binding molecule described herein comprises a second polynucleotide with a nucleotide sequence set forth in SEQ ID NO: 190. In one example, the expression system for an antigen-binding molecule described herein comprises a second polynucleotide with a nucleotide sequence set forth in SEQ ID NO: 191. In one example, the expression system for an antigen-binding molecule described herein comprises a second polynucleotide with a nucleotide sequence set forth in SEQ ID NO: 192. In one example, the expression system for an antigen-binding molecule described herein comprises a second polynucleotide with a nucleotide sequence set forth in SEQ ID NO: 193. In one example, the expression system for an antigen-binding molecule described herein comprises a second polynucleotide with a nucleotide sequence set forth in SEQ ID NO: 194. In one example, the expression system for an antigen-binding molecule described herein comprises a second polynucleotide with a nucleotide sequence set forth in SEQ ID NO: 195. In one example, the expression system for an antigen-binding molecule described herein comprises a second polynucleotide with a nucleotide sequence set forth in SEQ ID NO: 196. In one example, the expression system for an antigen-binding molecule described herein comprises a second polynucleotide with a nucleotide sequence set forth in SEQ ID NO: 197. In one example, the expression system for an antigen-binding molecule described herein comprises a second polynucleotide with a nucleotide sequence set forth in SEQ ID NO: 198. In one example, the expression system for an antigen-binding molecule described herein comprises a second polynucleotide with a nucleotide sequence set forth in SEQ ID NO: 199. In one example, the expression system for an antigen-binding molecule described herein comprises a second polynucleotide with a nucleotide sequence set forth in SEQ ID NO: 200. In one example, the expression system for an antigen-binding molecule described herein comprises a second polynucleotide with a nucleotide sequence set forth in SEQ ID NO: 201. In one example, the expression system for an antigen-binding molecule described herein comprises a second polynucleotide with a nucleotide sequence set forth in SEQ ID NO: 202. In one example, the expression system for an antigen-binding molecule described herein comprises a second polynucleotide with a nucleotide sequence set forth in SEQ ID NO: 203. In one example, the expression system for an antigen-binding molecule described herein comprises a second polynucleotide with a nucleotide sequence set forth in SEQ ID NO: 204. In one example, the expression system for an antigen-binding molecule described herein comprises a second polynucleotide with a nucleotide sequence set forth in SEQ ID NO: 205. In one example, the expression system for an antigen-binding molecule described herein comprises a second polynucleotide with a nucleotide sequence set forth in SEQ ID NO: 206. In one example, the expression system for an antigen-binding molecule described herein comprises a second polynucleotide with a nucleotide sequence set forth in SEQ ID NO: 207. In one example, the expression system for an antigen-binding molecule described herein comprises a second polynucleotide with a nucleotide sequence set forth in SEQ ID NO: 208. In one example, the expression system for an antigen-binding molecule described herein comprises a second polynucleotide with a nucleotide sequence set forth in SEQ ID NO: 209. In one example, the expression system for an antigen-binding molecule described herein comprises a second polynucleotide with a nucleotide sequence set forth in SEQ ID NO: 210. In one example, the expression system for an antigen-binding molecule described herein comprises a second polynucleotide with a nucleotide sequence set forth in SEQ ID NO: 211. In one example, the expression system for an antigen-binding molecule described herein comprises a second polynucleotide with a nucleotide sequence set forth in SEQ ID NO: 212. In one example, the expression system for an antigen-binding molecule described herein comprises a second polynucleotide with a nucleotide sequence set forth in SEQ ID NO: 213. In one example, the expression system for an antigen-binding molecule described herein comprises a second polynucleotide with a nucleotide sequence set forth in SEQ ID NO: 214. In one example, the expression system for an antigen-binding molecule described herein comprises a second polynucleotide with a nucleotide sequence set forth in SEQ ID NO: 215. In one example, the expression system for an antigen-binding molecule described herein comprises a second polynucleotide with a nucleotide sequence set forth in SEQ ID NO: 216. In one example, the expression system for an antigen-binding molecule described herein comprises a second polynucleotide with a nucleotide sequence set forth in SEQ ID NO: 217. In one example, the expression system for an antigen-binding molecule described herein comprises a second polynucleotide with a nucleotide sequence set forth in SEQ ID NO: 218. In one example, the expression system for an antigen-binding molecule described herein comprises a second polynucleotide with a nucleotide sequence set forth in SEQ ID NO: 219. In one example, the expression system for an antigen-binding molecule described herein comprises a second polynucleotide with a nucleotide sequence set forth in SEQ ID NO: 220. In one example, the expression system for an antigen-binding molecule described herein comprises a second polynucleotide with a nucleotide sequence set forth in SEQ ID NO: 221. In one example, the expression system for an antigen-binding molecule described herein comprises a second polynucleotide with a nucleotide sequence set forth in SEQ ID NO: 222. In one example, the expression system for an antigen-binding molecule described herein comprises a second polynucleotide with a nucleotide sequence set forth in SEQ ID NO: 223. In one example, the expression system for an antigen-binding molecule described herein comprises a second polynucleotide with a nucleotide sequence set forth in SEQ ID NO: 224. In one example, the expression system for an antigen-binding molecule described herein comprises a second polynucleotide with a nucleotide sequence set forth in SEQ ID NO: 225. In one example, the expression system for an antigen-binding molecule described herein comprises a second polynucleotide with a nucleotide sequence set forth in SEQ ID NO: 226. In one example, the expression system for an antigen-binding molecule described herein comprises a second polynucleotide with a nucleotide sequence set forth in SEQ ID NO: 227. In one example, the expression system for an antigen-binding molecule described herein comprises a second polynucleotide with a nucleotide sequence set forth in SEQ ID NO: 228. In one example, the expression system for an antigen-binding molecule described herein comprises a second polynucleotide with a nucleotide sequence set forth in SEQ ID NO: 229. In one example, the expression system for an antigen-binding molecule described herein comprises a second polynucleotide with a nucleotide sequence set forth in SEQ ID NO: 230. In one example, the expression system for an antigen-binding molecule described herein comprises a second polynucleotide with a nucleotide sequence set forth in SEQ ID NO: 231. In one example, the expression system for an antigen-binding molecule described herein comprises a second polynucleotide with a nucleotide sequence set forth in SEQ ID NO: 232. In one example, the expression system for an antigen-binding molecule described herein comprises a second polynucleotide with a nucleotide sequence set forth in SEQ ID NO: 233. In one example, the expression system for an antigen-binding molecule described herein comprises a second polynucleotide with a nucleotide sequence set forth in SEQ ID NO: 234. In one example, the expression system for an antigen-binding molecule described herein comprises a second polynucleotide with a nucleotide sequence set forth in SEQ ID NO: 235. In one example, the expression system for an antigen-binding molecule described herein comprises a second polynucleotide with a nucleotide sequence set forth in SEQ ID NO: 236. In one example, the expression system for an antigen-binding molecule described herein comprises a second polynucleotide with a nucleotide sequence set forth in SEQ ID NO: 237. In one example, the expression system for an antigen-binding molecule described herein comprises a second polynucleotide with a nucleotide sequence set forth in SEQ ID NO: 238. In one example, the expression system for an antigen-binding molecule described herein comprises a second polynucleotide with a nucleotide sequence set forth in SEQ ID NO: 239. In one example, the expression system for an antigen-binding molecule described herein comprises a second polynucleotide with a nucleotide sequence set forth in SEQ ID NO: 240. In one example, the expression system for an antigen-binding molecule described herein comprises a second polynucleotide with a nucleotide sequence set forth in SEQ ID NO: 241. In one example, the expression system for an antigen-binding molecule described herein comprises a second polynucleotide with a nucleotide sequence set forth in SEQ ID NO: 242. In one example, the expression system for an antigen-binding molecule described herein comprises a second polynucleotide with a nucleotide sequence set forth in SEQ ID NO: 243. In one example, the expression system for an antigen-binding molecule described herein comprises a second polynucleotide with a nucleotide sequence set forth in SEQ ID NO: 244. In one example, the expression system for an antigen-binding molecule described herein comprises a second polynucleotide with a nucleotide sequence set forth in SEQ ID NO: 245. In one example, the expression system for an antigen-binding molecule described herein comprises a second polynucleotide with a nucleotide sequence set forth in SEQ ID NO: 246. In one example, the expression system for an antigen-binding molecule described herein comprises a second polynucleotide with a nucleotide sequence set forth in SEQ ID NO: 247. In one example, the expression system for an antigen-binding molecule described herein comprises a second polynucleotide with a nucleotide sequence set forth in SEQ ID NO: 248. In one example, the expression system for an antigen-binding molecule described herein comprises a second polynucleotide with a nucleotide sequence set forth in SEQ ID NO: 249. In one example, the expression system for an antigen-binding molecule described herein comprises a second polynucleotide with a nucleotide sequence set forth in SEQ ID NO: 250. In one example, the expression system for an antigen-binding molecule described herein comprises a second polynucleotide with a nucleotide sequence set forth in SEQ ID NO: 251. In one example, the expression system for an antigen-binding molecule described herein comprises a second polynucleotide with a nucleotide sequence set forth in SEQ ID NO: 252. In one example, the expression system for an antigen-binding molecule described herein comprises a second polynucleotide with a nucleotide sequence set forth in SEQ ID NO: 253. In one example, the expression system for an antigen-binding molecule described herein comprises a second polynucleotide with a nucleotide sequence set forth in SEQ ID NO: 254. In one example, the expression system for an antigen-binding molecule described herein comprises a second polynucleotide with a nucleotide sequence set forth in SEQ ID NO: 255.

In one aspect, the present disclosure refers to a vector comprising the expression system disclosed herein. The term “vector” as used herein refers to a means, typically a nucleic acid, of transporting and expressing a target gene in a host cell. For example, the vector may include a plasmid vector, a cosmid vector, or a virus vector, such as a bacteriophage vector, an adenovirus vector, a retrovirus vector, and an adeno-associated virus vector. The recombinant vector may be prepared by manipulating a plasmid, a phage, or a virus known in the art.

In one aspect, the present disclosure refers to a host cell comprising the expression system disclosed herein or the vector disclosed herein. In one example, there is provided a host cell comprising the expression system or vector as disclosed herein. The host cell may be prokaryotic or eukaryotic host cells. The host cell, which is capable of stably and continuously cloning or expressing the expression system or vector, may be any host cell known in the art.

In one aspect, the present disclosure refers to a kit comprising the expression system disclosed herein, the vector disclosed herein, the host cell disclosed herein, additional buffers and/or reagents required for use of the kit, and/or instructions for use of the kit and any other relevant information. A skilled person familiar with molecular biology techniques would know or be able to determine the contents of the buffers and/or reagents commonly used in the art. In one example, the present disclosure refers to a kit comprising the expression system disclosed herein. In one example, the present disclosure refers to a kit comprising the vector disclosed herein. In one example, the present disclosure refers to a kit comprising the host cell disclosed herein. In one example, the expression system, the vector, or the host cell, provided in the kit as described herein may be provided in separate containers comprising the components independently distributed in one or more containers.

In one aspect, the present disclosure refers to the expression system disclosed herein, the vector disclosed herein, the host cell disclosed herein, or the kit disclosed herein for use in screening antibody libraries or antibody production. In one example, the present disclosure refers to the expression system disclosed herein for use in screening antibody libraries or antibody production. In one example, the present disclosure refers to the vector disclosed herein for use in screening antibody libraries or antibody production. In one example, the present disclosure refers to the host cell disclosed herein for use in screening antibody libraries or antibody production. In one example, the present disclosure refers to the kit disclosed herein for use in screening antibody libraries or antibody production. In one example, the present disclosure refers to the expression system disclosed herein, the vector disclosed herein, the host cell disclosed herein, or the kit disclosed herein for use in screening antibody libraries. In one example, screening antibody libraries involves, but is not limited to, identifying and selecting antibodies with desired properties, such as binding specificity or affinity. A person skilled in the art would know how to use methods known in the art such as, but not limited to, SDS-PAGE, ELISA, western blotting, flow cytometry, and immunohistochemistry, for screening antibody libraries using the expression system, the vector, the host cell, or the kit disclosed herein. In one example, the present disclosure refers to the expression system disclosed herein, the vector disclosed herein, the host cell disclosed herein, or the kit disclosed herein for use in antibody production. In one example, antibody production using an expression system, vector, host cell, or kit typically involves, but are not limited to, steps such as cloning the antibody genes into an expression vector, and culturing host cells for protein expression. A person skilled in the art would know how to use methods known in the art such as, but not limited to, antibody cloning, host cell transformation, antibody purification and quantification, for antibody production using the expression system, the vector, the host cell, or the kit disclosed herein.

In one aspect, the present disclosure refers to a method of producing one or more secretable antigen binding molecules and/or one or more membrane-bound antigen binding molecules, comprising culturing the host cell disclosed herein under suitable culture conditions such that one or more secretable antigen binding molecules and/or one or more membrane-bound antigen binding molecules are produced. In one example, the present disclosure refers to a method of producing one or more secretable antigen binding molecules and one or more membrane-bound antigen binding molecules, comprising culturing the host cell disclosed herein under suitable culture conditions such that one or more secretable antigen binding molecules and one or more membrane-bound antigen binding molecules are produced. In one example, the present disclosure refers to a method of producing one or more secretable antigen binding molecules comprising culturing the host cell disclosed herein under suitable culture conditions such that one or more secretable antigen binding molecules are produced. In one example, the present disclosure refers to a method of producing one or more membrane-bound antigen binding molecules, comprising culturing the host cell disclosed herein under suitable culture conditions such that one or more membrane-bound antigen binding molecules are produced.

In one example, the method of producing one or more secretable antigen binding molecules and/or one or more membrane-bound antigen binding molecules disclosed herein, further comprises recovering the one or more secretable antigen binding molecules. In one example, the method of producing one or more secretable antigen binding molecules disclosed herein, further comprises recovering the one or more secretable antigen binding molecules. In one example, the method of producing one or more membrane-bound antigen binding molecules disclosed herein, further comprises recovering the one or more secretable antigen binding molecules. In one example, recovering secretable antigen-binding molecules typically involves, but are not limited to, isolating and purifying the molecules from the culture medium or cellular extracts. A person skilled in the art would be well-versed in conventional methods for recovering antigen-binding molecules such as, but not limited to, protein affinity chromatography, ion exchange chromatography, size-exclusion chromatography, precipitation and ultrafiltration.

In one aspect, the present disclosure refers to a method for detecting the presence of one or more secretable antigen binding molecules or one or more membrane-bound antigen binding molecules, or one or more secretable antigen binding molecules and one or more membrane-bound antigen binding molecules, the method comprising:
providing an expression system disclosed herein;
delivering said expression system to one or more target cells;
   wherein said target cells transcribe said expression system into one or more amino acid sequences,
   wherein when all of the cleavage sites in the one or more amino acid sequences are cleaved, one or more secretable antigen binding molecules comprising the first and second parts of the antigen binding molecules are secreted by the target cells,
   wherein when the first and second cleavage sites of the one or more amino acid sequences are cleaved and the third cleavage site of the one or more amino acid sequences is not cleaved, one or more membrane-bound antigen binding molecules comprising the first and second parts of the antigen binding molecules are bound to the surface of the target cells; and
detecting the presence or absence of the one or more secretable antigen binding molecules and/or one or more membrane-bound antigen binding molecules.

In one example, the present disclosure refers to a method for detecting the presence of one or more secretable antigen binding molecules and/or one or more membrane-bound antigen binding molecules disclosed herein, wherein:
(i) the presence or absence of the one or more secretable antigen binding molecules is detected using enzyme-linked immunosorbent assay (ELISA) or alternative binding assay; and/or
(ii) the presence or absence of the one or more membrane-bound antigen binding molecules is detected using flow cytometry analysis, wherein the first part of the antigen binding molecules, the second part of the antigen binding molecules and/or an antigen specific to the antigen binding molecules are stained prior to the flow cytometry analysis. In one example, the one or more secretable antigen binding molecules and/or one or more membrane-bound antigen binding molecules can be detected using any conventional methods known in the art. A person skilled in the art would be well-versed in conventional methods for detecting secretable antigen binding molecules such as, but not limited to, SDS-PAGE, ELISA, western blotting, immunofluorescence, protein microarray, and surface plasmon resonance. A person skilled in the art would also be well-versed in conventional methods for detecting membrane-bound antigen binding molecules such as, but not limited to, flow cytometry, immunohistochemistry, immunoprecipitation, surface plasmon resonance, SDS-PAGE, western blotting, and Fluorescence-Activated Cell Sorting (FACS). A person skilled in the art would readily understand that flow cytometry analysis involves passing cells labeled with fluorescent markers through a laser beam, detecting emitted fluorescence and scattered light using sensors. Standard laboratory protocols and commercially available flow cytometers are well-known to those skilled in the art, for performing the flow cytometry analysis for various applications, including immunophenotyping, cell sorting, and the study of diverse cellular functions.

As used in this application, the singular form “a,” “an,” and “the” include plural references unless the context clearly dictates otherwise. For example, the term “a primer” includes a plurality of primers, including mixtures and combinations thereof.

As used herein, the terms “increase” and “decrease” refer to the relative alteration of a chosen trait or characteristic in a subset of a population in comparison to the same trait or characteristic as present in the whole population. An increase thus indicates a change on a positive scale, whereas a decrease indicates a change on a negative scale. The term “change”, as used herein, also refers to the difference between a chosen trait or characteristic of an isolated population subset in comparison to the same trait or characteristic in the population as a whole. However, this term is without valuation of the difference seen.

As used herein, the term “about” in the context of concentration of a substance, size of a substance, length of time, or other stated values means+/−5% of the stated value, or +/−4% of the stated value, or +/−3% of the stated value, or +/−2% of the stated value, or +/−1% of the stated value, or +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The disclosure illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the disclosures embodied therein herein disclosed may be resorted to by those skilled in the art, and that such modifications and variations are considered to be within the scope of this disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

Other embodiments are within the following claims and non-limiting examples. In addition, where features or aspects of the disclosure are described in terms of Markush groups, those skilled in the art will recognize that the disclosure is also thereby described in terms of any individual member or subgroup of members of the Markush group.

### EXAMPLES

### Methods

### Cell Culture and Media for Maintenance of CHO K1 Master Cell Line (MCL)

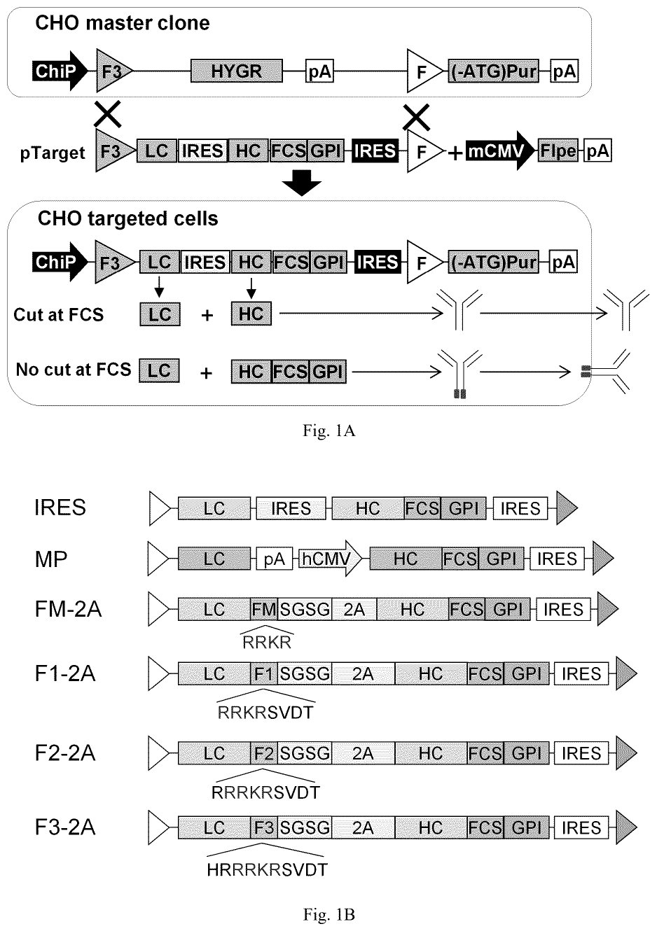

The CHO K1 master cell line (MCL) was generated by nucleofection of a landing pad vector into CHO K1 cells (ATCC), followed by screening clones for single copy integration by southern blotting. The landing pad vector expressed a hygromycin resistant gene (HYG) using a chimeric promoter (ChiP) which consisted of the murine CMV enhancer (M11788), the hCMV core promoter and the hCMV intron A (M60321). The HYG expression cassette was flanked by FRT3 and FRT. An impaired puromycin resistant gene lacking start codon ((ATG-) Puro) followed by the simian virus 40 (SV40) polyadenylation signal (pA) was placed downstream of FRT for selecting correct cassette exchange by RMCE (FIG. 1A). It was confirmed that the MCL contained only one copy of landing pad vector at a single integration site by southern blotting and targeted locus amplification (TLA) analyses (Cergentis). The MCL was grown in a protein-free medium (maintenance media) consisting of 50% HyQ PF (GE Healthcare Life Sciences) and 50% CD CHO (ThermoFisher) supplemented with 1 g/L sodium carbonate (Sigma), 6 mM glutamine (Sigma) and 0.1% Pluronic F-68 (Thermo Fisher) in a humidified Kuhner shaker (Adolf Kühner AG) with 8% CO₂ at 37° C. Routine subculture was conducted every three to four days by seeding cells at density of 3×10⁵ cells/mL in 15 mL of fresh medium in 125 mL shake flasks (Corning). Cell density and viability were determined by trypan blue exclusion method on Vi-Cell XR viability analysers (Beckman Coulter).

Generating Stable mAb-Producing Cell Lines Via Recombinase-Mediated-Cassette-Exchange (RMCE) and Random Integration

The MCL was co-transfected with an appropriate targeting vector and a vector expressing FLPe using Amaxa SG Cell Line 4D-Nucleofector® X Kit and program FF-137 (Lonza). In each transfection, 1×10⁷ cells were transfected with 5 μg of targeting plasmid vector and 5 μg of FLPe plasmid vector in circular format. The transfected cells were then re-suspended in 2 mL of maintenance media preloaded in 6-well suspension culture plates (NUNC™) and incubated in static incubators (IncuSafe, Sanyo). At 24 hour post-transfection, they were collected by centrifugation (100×g, 5 min) and re-suspended in 15 mL of protein-free maintenance medium in 125 mL shake flasks in the humidified Kuhner shaker (Adolf Kühner AG) with 8% CO₂ at 37° C. Four days later, the transfected cells were subjected to selection in the maintenance media containing Puromycin (InvivoGen) at 20 μg/mL. Selection was continued for two weeks by passaging in the selection medium every three to four days. Stably transfected cell pools were deemed established when cell viabilities recovered over 95%.

The protocol for further transfection of the three multi-cistronic vectors expressing B4GALT1, ST6GAL1 and the combination of these two genes respectively into the targeted pools already expressing either the combination of B4GALT1 and ST6GAL1 or MGAT5 was the same as that described for RMCE with slight modifications. In each transfection 5 μg of linearized plasmids were transfected to 1×10⁷ cells. After incubating in 2 mL of protein-free medium in the 6-well suspension culture plates (NUNC™) overnight, the transfected cells were collected by centrifuge at 100×g for 5 minutes and then resuspended in 15 mL of protein-free medium supplemented with blasticidin (Thermofisher Scientific) at 20 μg/mL. Passaging in selection medium was subsequently carried out every three to four days until cell viabilities recovered over 95%.

### Characterization of Growth and Productivity of Stable Pools

Stable cell pools were subjected to seven-day fed-batch production by seeding 30 mL of cultures at viable cell density of 3×10⁵ cells/mL in 50 ml tube spin (TPP) in the humidified Kuhner shaker (Adolf Kühner AG) with 8% CO₂ at 37° C. 3 mL of Ex-Cell Advanced CHO Feed 1 (with glucose) (SAFC, Sigma) and 400 μL 45% (w/v) D-glucose (Sigma) were added at day five. Cell density, viability and antibody titer were monitored at day three, five and seven using the Vi-Cell XR viability analyzer (Beckman Coulter) and an IMMAGE 800 immunochemistry system (Beckman Coulter), respectively. The IMMAGE 800 immunochemistry system utilized anti-human Fc region antibodies for IgG quantification. The specific mAb productivity (qP) in the exponential phase of cultures was calculated as the difference in mAb concentration between day five and seven divided by the integrated viable cell density (IVCD) which was determined based on the trapezoidal method. Two sets of ten million cells were collected from each culture at day five for analysis of mRNA and protein levels, respectively. Flow cytometry was performed at day five on BD FACSCalibur to determine the homogenous expression of the DsRed protein in the stable pools. Flow cytometry data were analyzed using FlowJo software. Culture supernatant was harvested at day seven, by centrifuging at 5000×g over 10 min to remove cells and use for N-glycan analysis.

### SDS-PAGE

The purified samples were subjected to SDS-PAGE, followed by Coomassie Blue staining to assess the presence of different species in a sample. 2-3 μg of each sample were resolved on the NuPAGE™ 4-12% Bis-Tris Protein Gels (Thermo Fisher Scientific) in MOPS buffer following manufacturer's protocol under both reducing and non-reducing conditions. The gels were then incubated in the fixing buffer (50% Methanol & 10% Acetic acid) for 10 mins, and then followed by 10-minutes staining in 0.1% Coomassie blue staining solution on an orbital shaker. De-staining was performed using 30% ethanol until the background is clear. Gel photos were taken with the ChemiDoc Imaging System (Biorad).

### Intact MS

The molecular masses of all samples were measured by a quadrupole time-of-flight (QTOF) mass analyzer coupled to a reversed-phase LC system. The analysis was carried out by directly injecting 4 μL of each sample (125 ng/μL) into a C4 column coupled to the QTOF mass spectrometer, and the acquisition of mass spectrum was set at the mass range between 1000 m/z to 4000 m/z. The column temperature was maintained at 60° C. The bound mAb was eluted at a 50 μL/minute flow rate using a LC gradient shown in Table 3 over 12 minutes. In the 12 minute LC gradient, the first 3 minute was on-column desalting followed by 9 minute of linear gradient for mAb separation and column re-equilibration. The MS method was built in two periods: 3 minute with ion spray voltage floating (ISVF) set to 0 V during on-column desalting to avoid spraying salt into MS, followed by 9 minute with ISVF set to 5500 V for sample analysis. MS data processing, including spectral deconvolution, mass reconstruction and analysis of glycoforms and other PTMs, was performed using Protein Metrics Intact Mass Software V3.1-19. Peaks representing major glycoforms or subunits and the corresponding molecular mass were derived from the deconvoluted mass spectra for biosimilar and reference lots. The relative ratios of major glycoforms and subunit peaks (i.e. relative intensity of each peak in percentage) for each lot were defined as the intensity relative to the highest intensity peak in each individual sample lot.

### Flow Cytometry

The targeted cells were stained with anti-human IgG (y-chain specific) FITC conjugate for quantification of bound antibodies on the cell surface using flow cytometry.

### Results

Integration of IRES-Mediated Targeting Vector, MP-Mediated Targeting Vector, and 2A Peptides-Mediated Targeting Vector into the Landing Pad in the CHO Master Clone

A CHO targeted integration platform for high throughput screening of antibody libraries was developed as basis for integration of the IRES-mediated targeting vector, MP-mediated targeting vector, and 2A peptides-mediated targeting vector (FIG. 1A). The CHO targeted integration platform consisted of two key components: 1) a CHO master clone containing a single copy landing pad in a genomic site that provides stable and high-level gene expression; and 2) a targeting vector which allowed simultaneous display and secretion of antibodies. The targeting vector used IRES to express the antibody light chain and antibody heavy chain in one transcript. Simultaneous display and secretion of antibodies were achieved by linking a glycosidylphosphatidylinositol membrane anchor (GimmunoPI) to the C-terminus of the heavy chain through an engineered FCS with incomplete cleavage efficiency. Integration of single-copy targeting vectors carrying different antibody genes into the landing pad in each CHO master cell was achieved through either FLP/FRT-based RMCE or CRISPR knock-in. It was demonstrated that the CHO targeted integration platform enables screening of antibody libraries consisting of over millions of molecules and produces secreted antibodies with titers around 200 mg/L in fed-batch cultures. The CHO targeted integration platform provided a powerful tool for enhancing the efficiency of antibody discovery and engineering. However, the low antibody titer provided by the CHO targeted integration platform itself limits its wider application in developability and function studies.

When the IRES-mediated targeting vector was integrated into the landing pad in the CHO master clone, the light chain gene was under the control of the built-in promoter and translated by the canonical cap-dependent mechanism while the heavy chain gene was driven by IRES and translated through a cap-independent mechanism. The IRES-driven cap-independent translation had lower efficiency than the cap-dependent translation, resulting in low heavy chain gene expression and thus low antibody titers. To further enhance the secreted antibody titers, the inventors of the present disclosure compared the IRES, MP and 2A peptides for co-expression of light chain and heavy chain in the context of the CHO targeted integration platform (FIG. 1B). When expressing multiple genes in one vector using MP, each gene was driven by its own promoter and transcribed separately. The potential drawback of using an MP-mediated targeting vector was that transcriptional interference, where an active transcriptional unit suppressed the expression of another unit, may result in lower expression of one gene than the other gene. In contrast, 2A peptides-linked light chain and heavy chain were expressed in one single open reading frame and “self-cleavage” occurred co-translationally between the last two amino acids, GP, at the C-terminus of the 2A polypeptide, giving rise to equal amounts of light chain and heavy chain polypeptides, which were beneficial for enhancing antibody expression levels. It is well-known in the art that Furin is a ubiquitous subtilisin-like proprotein convertase with a minimal cleavage site of R-X-R/K-R and the cleavage of proteins occurs in Golgi. To remove 2A residues which would otherwise be attached to the light chain, a minimal FCS (Fm), RRKR, was inserted upstream of the 2A peptide. In addition, an SGSG linker was inserted between Fm and 2A peptide to enhance the cleavage efficiency of 2A peptide.

Comparative Data of Cells Transfected with IRES, MP, Fm-2A, F1-2A, F2-2A and F3-2A Targeting Vectors

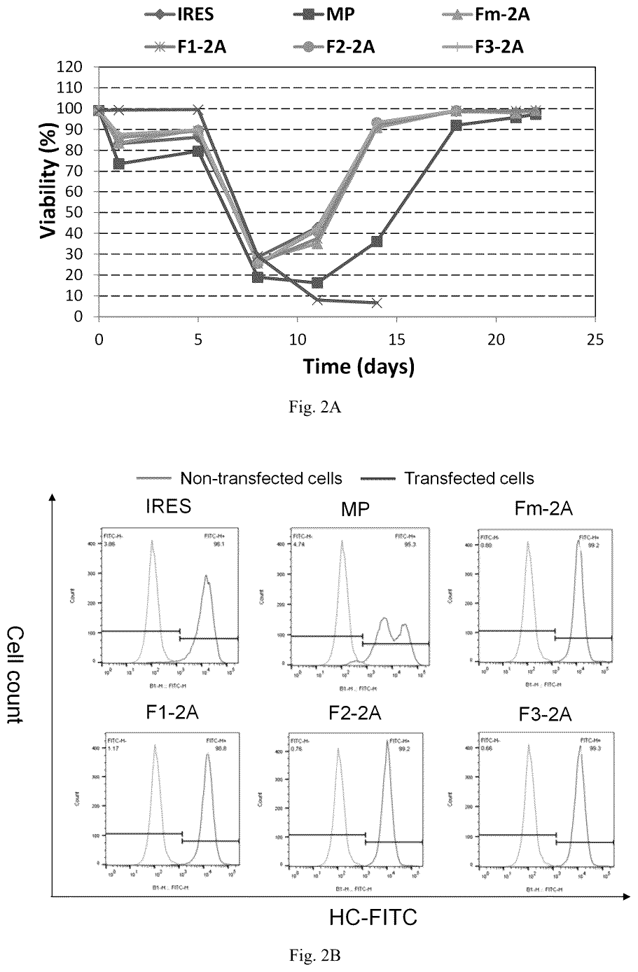

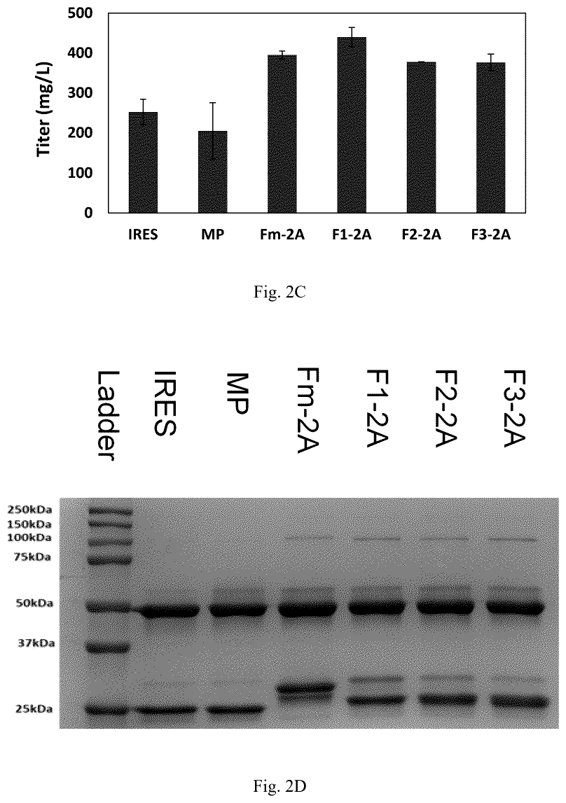

Each of the three targeting vectors, IRES, MP and Fm-2A, was co-transfected with a vector expressing Flpe into the CHO master clone. The transfected cells were incubated in the incubator over 5 days to allow RMCE occurrence. The transfected cells were subsequently passaged in medium containing puromycin to select for transfected cells in which the targeting vector correctly integrated into the landing pad. When the pools of transfected cells were recovered, they were stained with anti-human IgG (y-chain specific) FITC conjugate for quantification of bound antibodies on the cell surface by flow cytometry. The stably transfected pools for each targeting vector were also characterized for productivity in 14-day fed-cultures. The culture supernatant was harvested at the end of fed-batch and purified by protein A. The purified antibodies for each targeting vector were analyzed using SDS PAGE under reducing conditions. The viability of transfected cells gradually dropped and then slowly picked up when the transfected cells were passaged in the selection medium. The pools of cells transfected with IRES and Fm-2A targeting vectors had their viabilities recovered over 95% in 18 days while the pools generated using MP targeting vector took three extra days to recover (FIG. 2A). Flow cytometry analysis of the antibodies displayed on the cell surface indicated that IRES and Fm-2A vectors gave homogeneous expression, while the pool of cells generated using MP vector had heterogeneous expression indicated by two peaks in the histogram (FIG. 2B). Compared to IRES-mediated targeting vector, the MP vector gave slightly lower titer in fed-batch cultures, while Fm-2A enhanced the secreted antibody titer by about one-fold (FIG. 2C). However, the light chain polypeptides expressed from Fm-2A had bigger size than those from IRES and MP vectors. MS analysis confirmed that 2A residues were attached to the light chain, indicating cleavage at Fm was not successful (FIG. 3B).

The amino acids flanking the minimal cleavage site of R-X-R/K-R affected the furin cleavage efficiency. The inventors of the present disclosure designed three FCS variants, F1, F2 and F3 to include the conserved amino acids around RRKR in the 2A targeting vector to enhance cleavage efficiency (FIG. 1B). The three new design targeting vectors, F1-2A, F2-2A and F3-2A took similar time to generate stable pools compared to the IRES targeting vector (FIG. 2A). The antibodies displayed on the cell surface from these three 2A vectors were homogenous (FIG. 2B). In fed-batch cultures, F1-2A, F2-2A and F3-2A gave similar antibody titers as that from the Fm-2A vector. SDS-PAGE analysis indicated F1-2A, F2-2A and F3-2A produced light chain polypeptides with reduced sizes compared to that from the Fm-2A and similar size as that expressed from IRES and MP targeting vectors (FIG. 2D). Further analysis of the molecular weight found out that the product expressed from F1-2A contained 60% of light chain polypeptides with 2A removed, while those from F2-2A and F3-2A had 90% of light chain polypeptides with 2A removed. However, the light chain polypeptides from F3-2A were heterogeneous due to the attachment of different number of amino acids residues from furin cleavage sequence. Overall, F2-2A was the best for co-expression of antibody light chain and heavy chain in terms of secreted antibody titers and quality.

### Evaluation of Cleavage Efficiency of FCS Variants

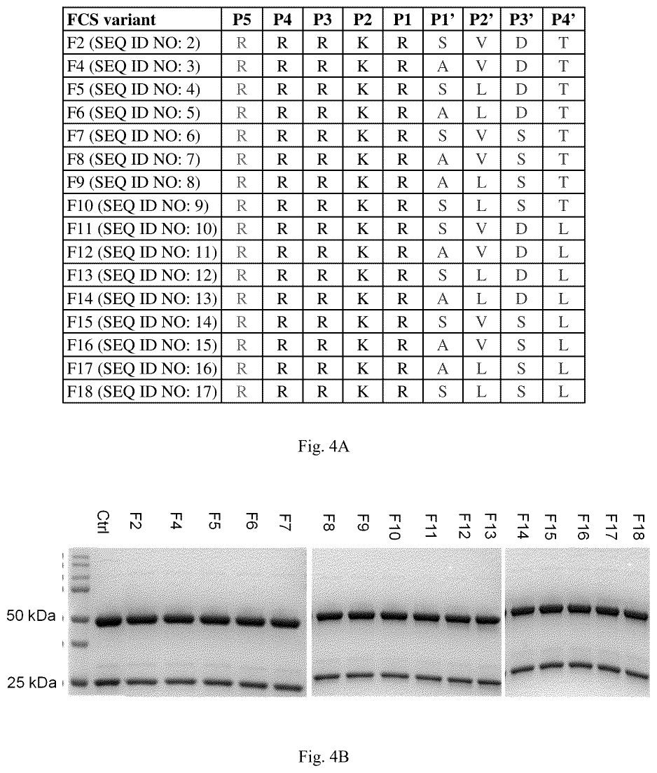

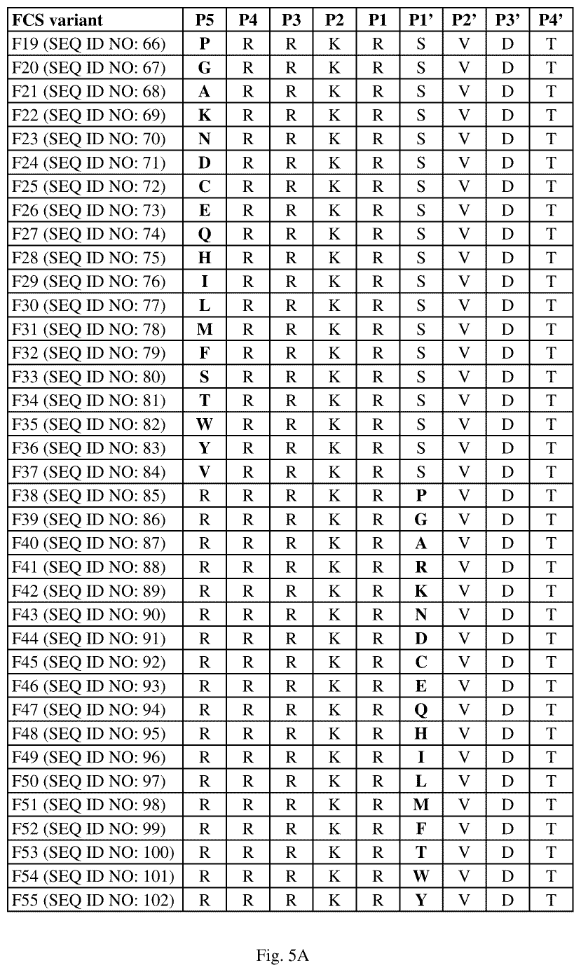

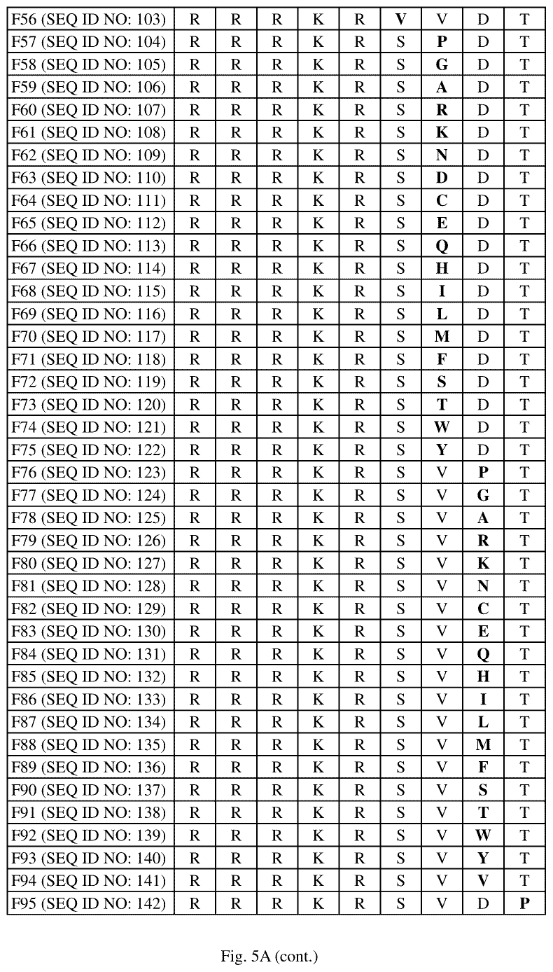

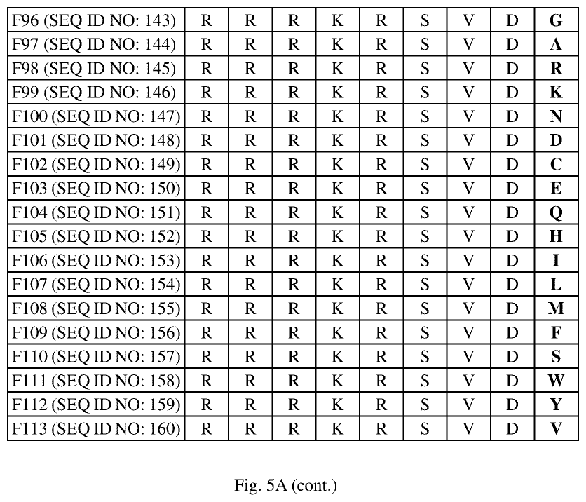

The inventors further designed another 15 FCS variants, F4 to F18 to have RRKR flanked with R at P5, S/A at P1′, V/L at P2′, D/S at P3′ and T/L at P4′ (FIG. 4A). Combination of these 15 variants and 2A were tested for co-expression of light chain and heavy chain in the dual display and secretion targeting vectors using the same procedure as described above. SDS-PAGE analysis indicated that the light chain and heavy chain polypeptides expressed from these FCS variants-2A had same sizes as those expressed from the control IRES vector (FIG. 4B).

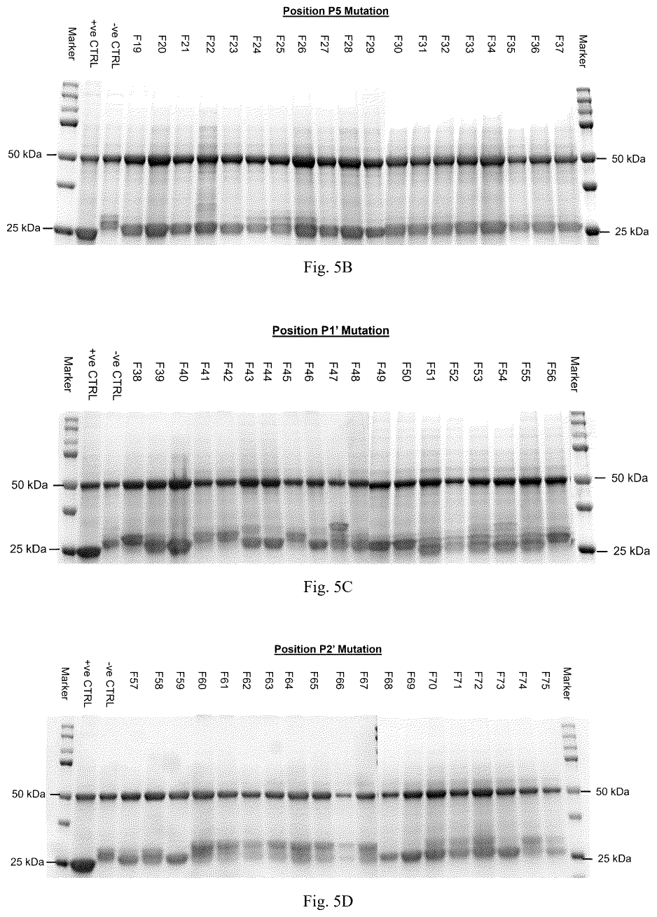

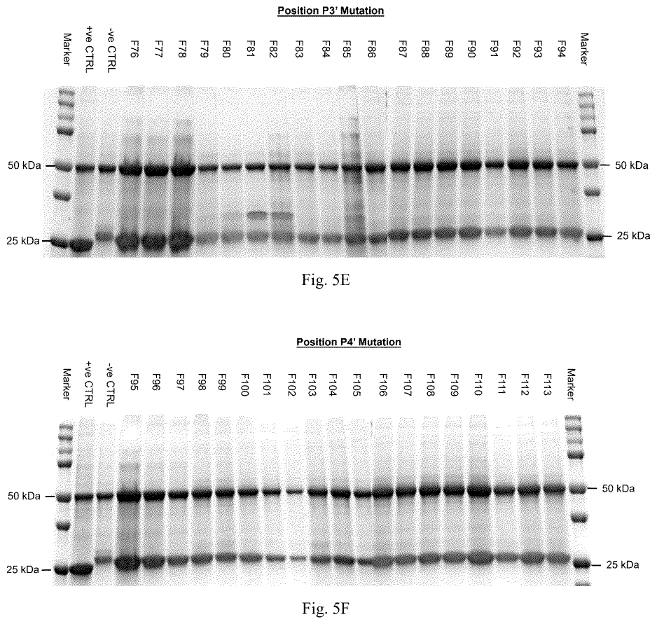

To investigate the influence of distinct amino acid residues at individual positions within the furin cleavage sequence, the inventors designed 95 variants, altering one amino acid at a time in the RRRKRSVDT (SEQ ID NO: 2) sequence while keeping other amino acids constant (FIG. 5A). These variants underwent testing for the co-expression of antibody light chain and antibody heavy chain in both the dual display and secretion targeting vectors, following the procedure as described above in the Methods section Notably, substituting the amino acid at the P5 position from Arginine to Aspartic acid (R to D) [FCS variant F24], Arginine to Cysteine (R to C) [FCS variant F25], or Arginine to Glutamic acid (R to E) [FCS variant F26] resulted in inefficient cleavage, whereas altering it to other amino acid types did not affect cleavage efficiency (FIG. 5B). At the P1′ position, replacing Serine(S) with Alanine (A) [FCS variant F40], Aspartic acid (D) [FCS variant F44], or Glutamic acid (E) [FCS variant F46] maintained high cleavage efficiency, while substituting it with other amino acids led to decreased cleavage efficiency. Similarly, at the P2′ position, substituting Valine (V) with Leucine (L) [FCS variant F69], Threonine (T) [FCS variant F73], Isoleucine (I) [FCS variant F68], or Alanine (A) [FCS variant F59] maintained high cleavage efficiency, while substitution with other amino acids resulted in decreased cleavage efficiency. Mutations at P3′ and P4′ did not impact cleavage efficiency. FIG. 4 and FIG. 5 show that having amino acids Serine(S), Alanine (A), Aspartic acid (D), and Glutamic acid (E) at P1′ position, amino acids Valine (V), Leucine (L), Threonine (T), Isoleucine (I), and Alanine (A) at P2′ position, and all 20 amino acids except Aspartic acid (D), Cysteine (C), and Glutamic acid (E) at P5 position are most conducive to enhancing cleavage efficiency. Any combinations of these amino acids at P5, P1′ and P2′ positions in FCS variants with R-X-(K/R)-R at P4, P3, P2, P1 positions, respectively, result in FCS variants with high cleavage efficiency. It was found that when a combination of amino acids that provide high cleavage efficiency is used at positions P5, P1′, and P2′ in FCS variants with R-X-(K/R)-R at P4, P3, P2, P1 positions, respectively, using any amino acids at positions P3′ and P4′ still results in FCS variants with high cleavage efficiency.

### Discussion

Displaying full length mAbs on the surface of mammalian cells requires tightly coupled co-expression of light chain and heavy chain genes in one single vector. This can be achieved by the use of MP, IRES and the combination of furin cleavage sequence and 2A peptides (F-2A). Compared to IRES and MP, F-2A linked genes are expressed in one single open reading frame, giving rise to equal amounts of the different genes. The inventors of the present disclosure compared the IRES, MP and F-2A peptide for co-expression of antibody light chain and heavy chain for obtaining simultaneous display and secretion of antibodies in a CHO targeted integration platform. In each targeting vector, the antibody light chain was arranged as the first gene, followed by either IRES, MP or F-2A to drive the expression of heavy chain. The antibody heavy chain was further linked to a membrane anchor through an engineered furin cleavage sequence with decreased cleavage efficiency. To remove 2A residues which would otherwise be attached to the light chain expressed from the 2A targeting vector, a minimal furin cleavage sequence (Fm), RRKR, was inserted upstream of the 2A peptide. In addition, a SGSG linker was inserted between Fm and 2A to enhance the cleavage efficiency of 2A peptide. Fm-2A provided enhanced level of antibody secretion and homogeneity of antibody display on the cell surface compared to the use of IRES and MP. However, the majority of secreted antibodies had 2A residues attached to the C-terminus of light chain polypeptides, indicating inefficient cleavage at Fm. To overcome this issue, the inventors designed engineered furin cleavage sequence variants with highly conserved amino acids flanking RRKR. The combination of these newly designed furin cleavage sequence variants with 2A peptides enhanced the secretion of antibodies with correct size without compromising the level of secreted antibody titers and homogeneity of antibody display.

The amino acids flanking the minimal furin cleavage sequence R-X-R/K-R affected furin cleavage efficiency. A huge number of tests needed to be done to identify the types of amino acids and particular combinations for enhancing the furin cleavage efficiency. The inventors of the present disclosure found that the addition of amino acids around R-X-R/K-R in naturally found furin cleavage sequence was able to enhance the cleavage efficiency. Accordingly, the design of furin cleavage sequence based on this principle showed improved furin cleavage efficiency.

Notable features of the method disclosed herein include:
1. The expression vector described herein that comprises at least the following domains:
   Light chain variable region of an antibody;
   Z-R-X1-X2-R-X3-X4-X5-X6;
   2A; and
   Heavy chain variable region of an antibody.
2. Plasmid expression vectors containing engineered furin cleavage sequences for co-expression of multiple genes, such as light chain and heavy chain of mAbs at high levels and homogeneously across cells.

The expression system of the present disclosure has the following advantages:
1. The expression system described herein is capable of producing higher levels of antibodies for cell surface display (membrane-bound) and secretable antibodies compared to conventional methods known in the art.
2. The expression system described herein is capable of producing secretable antibodies with high homogeneity compared to conventional methods known in the art.
3. An improved expression system with high cleavage efficiency capable of co-expression of antibody light chains and heavy chains for the screening of antibody libraries, providing a more efficient process for the development of therapeutic antibodies, compared to expression systems known in the art.
4. An improved expression system with high cleavage efficiency capable of co-expression of antibody light chains and heavy chains, that can more efficiently generate therapeutically specific and functional antibody proteins for the development of mRNA-based therapies.

SEQUENCE LISTING

| Sequence Name | Sequence | Polynucleotide sequence |
|---|---|---|
| General | R-R-X1-X2-R-X3-X4- | |
| sequence of a | X5-X6 (SEQ ID NO: 1) | |
| first cleavage site | | |
| FCS variant F2 | R-R-R-K-R-S-V-D-T | CGTAGACGGAAACGCAGCGTTGACAC |
| | (SEQ ID NO: 2) | C (SEQ ID NO: 35) |
| FCS variant F4 | R-R-R-K-R-A-V-D-T | CGTAGACGGAAACGCGCCGTTGACAC |
| | (SEQ ID NO: 3) | C (SEQ ID NO: 36) |
| FCS variant F5 | R-R-R-K-R-S-L-D-T | CGTAGACGGAAACGCAGCCTCGACAC |
| | (SEQ ID NO: 4) | C (SEQ ID NO: 37) |
| FCS variant F6 | R-R-R-K-R-A-L-D-T | CGTAGACGGAAACGCGCCCTCGACAC |
| | (SEQ ID NO: 5) | C (SEQ ID NO: 38) |
| FCS variant F7 | R-R-R-K-R-S-V-S-T | CGTAGACGGAAACGCAGCGTTTCTACC |
| | (SEQ ID NO: 6 | (SEQ ID NO: 39) |
| FCS variant F8 | R-R-R-K-R-A-V-S-T | CGTAGACGGAAACGCGCCGTTTCTAC |
| | (SEQ ID NO: 7) | C (SEQ ID NO: 40) |
| FCS variant F9 | R-R-R-K-R-A-L-S-T | CGTAGACGGAAACGCGCCCTCTCTAC |
| | (SEQ ID NO: 8) | C (SEQ ID NO: 41) |
| FCS variant F10 | R-R-R-K-R-S-L-S-T | CGTAGACGGAAACGCTCCCTCTCTACC |
| | (SEQ ID NO: 9) | (SEQ ID NO: 42) |
| FCS variant F11 | R-R-R-K-R-S-V-D-L | CGTAGACGGAAACGCAGCGTTGACCT |
| | (SEQ ID NO: 10) | C (SEQ ID NO: 43) |
| FCS variant F12 | R-R-R-K-R-A-V-D-L | CGTAGACGGAAACGCGCCGTTGACCT |
| | (SEQ ID NO: 11) | C (SEQ ID NO: 44) |
| FCS variant F13 | R-R-R-K-R-S-L-D-L | CGTAGACGGAAACGCAGCCTCGACCT |
| | (SEQ ID NO: 12) | C (SEQ ID NO: 45) |
| FCS variant F14 | R-R-R-K-R-A-L-D-L | CGTAGACGGAAACGCGCCCTCGACCT |
| | (SEQ ID NO: 13) | C (SEQ ID NO: 46) |
| FCS variant F15 | R-R-R-K-R-S-V-S-L | CGTAGACGGAAACGCAGCGTTTCTCTC |
| | (SEQ ID NO: 14) | (SEQ ID NO: 47) |
| FCS variant F16 | R-R-R-K-R-A-V-S-L | CGTAGACGGAAACGCGCCGTTTCTCTC |
| | (SEQ ID NO: 15) | (SEQ ID NO: 48) |
| FCS variant F17 | R-R-R-K-R-A-L-S-L | CGTAGACGGAAACGCGCCCTCTCTCT |
| | (SEQ ID NO: 16) | C (SEQ ID NO: 49) |
| FCS variant F18 | R-R-R-K-R-S-L-S-L | CGTAGACGGAAACGCTCTCTCTCTCTC |
| | (SEQ ID NO: 17) | (SEQ ID NO: 50) |
| Furin consensus | R-X-K-R | |
| sequence | (SEQ ID NO: 18) | |
| Furin consensus | R-X-R-R | |
| sequence | (SEQ ID NO: 19) | |
| P2A polypeptide | ATNFSLLKQAGDVEE | GCTACTAACTTCAGCCTGCTGAAGCAG |
| | NPGP (SEQ ID NO: 20) | GCTGGAGACGTGGAGGAGAACCCTGG |
| | | GCCC (SEQ ID NO: 51) |
| F2A polypeptide | APVKQTLNFDLLKLAG | GCACCGGTGAAACAGACTTTGAATTTT |
| | DVESNPGP (SEQ ID | GACCTTCTGAAGTTGGCAGGAGACGTT |
| | NO: 21) | GAGTCCAACCCTGGGCCC (SEQ ID NO: |
| | | 52) |
| E2A polypeptide | QCTNYALLKLAGDVE | CAGTGTACTAATTATGCTCTCTTGAAAT |
| | SNPGP (SEQ ID NO: | TGGCTGGAGATGTTGAGAGCAACCCT |
| | 22) | GGGCCC (SEQ ID NO: 53) |
| T2A polypeptide | EGRGSLLTCGDVEEN | GAGGGCAGAGGAAGTCTGCTAACATG |
| | PGP (SEQ ID NO: 23) | CGGTGACGTCGAGGAGAATCCTGGGC |
| | | CC (SEQ ID NO: 54) |
| Cleaved T2A | EGRGSLLTCGDVEEN | GAGGGCAGAGGAAGTCTGCTAACATG |
| polypeptide | PG (SEQ ID NO: 24) | CGGTGACGTCGAGGAGAATCCTGGG |
| | | (SEQ ID NO: 55) |
| First 5 amino | ATNFS (SEQ ID NO: | GCTACTAACTTCAGC (SEQ ID NO: 56) |
| acids of P2A | 25 | |
| A1P mutant | PTNFS (SEQ ID NO: | CCTACTAACTTCAGC (SEQ ID NO: 57) |
| | 26) | |
| A1G mutant | GTNFS (SEQ ID NO: | GGCACTAACTTCAGC (SEQ ID NO: 58) |
| | 27) | |
| T2G mutant | AGNFS (SEQ ID NO: | GCTGGCAACTTCAGC (SEQ ID NO: 59) |
| | 28) | |
| T2P mutant | APNFS (SEQ ID NO: | GCTCCTAACTTCAGC (SEQ ID NO: 60) |
| | 29) | |
| N3P mutant | ATPFS (SEQ ID NO: | GCTACTCCTTTCAGC (SEQ ID NO: 61) |
| | 30) | |
| N3A mutant | ATAFS (SEQ ID NO: | GCTACTGCTTTCAGC (SEQ ID NO: 62) |
| | 31) | |
| F4P mutant | ATNPS (SEQ ID NO: | GCTACTAACCCTAGC (SEQ ID NO: 63) |
| | 32) | |
| F4A mutant | ATNAS (SEQ ID NO: | GCTACTAACGCTAGC (SEQ ID NO: 64) |
| | 33) | |
| S5P mutant | ATNFP (SEQ ID NO: | GCTACTAACTTCCCT (SEQ ID NO: 65) |
| | 34) | |
| FCS variant F19 | P-R-R-K-R-S-V-D-T | CCTAGACGGAAACGCAGCGTTGACAC |
| | (SEQ ID NO. 66) | C |
| | | (SEQ ID NO: 161) |
| FCS variant F20 | G-R-R-K-R-S-V-D-T | GGCAGACGGAAACGCAGCGTTGACAC |
| | (SEQ ID NO. 67) | C |
| | | (SEQ ID NO: 162) |
| FCS variant F21 | A-R-R-K-R-S-V-D-T | GCCAGACGGAAACGCAGCGTTGACAC |
| | (SEQ ID NO. 68) | C |
| | | (SEQ ID NO: 163) |
| FCS variant F22 | K-R-R-K-R-S-V-D-T | AAGAGACGGAAACGCAGCGTTGACAC |
| | (SEQ ID NO. 69) | C (SEQ ID NO: 164) |
| FCS variant F23 | N-R-R-K-R-S-V-D-T | AACAGACGGAAACGCAGCGTTGACAC |
| | (SEQ ID NO. 70) | C (SEQ ID NO: 165) |
| FCS variant F24 | D-R-R-K-R-S-V-D-T | GACAGACGGAAACGCAGCGTTGACAC |
| | (SEQ ID NO. 71) | C (SEQ ID NO: 166) |
| FCS variant F25 | C-R-R-K-R-S-V-D-T | TGTAGACGGAAACGCAGCGTTGACAC |
| | (SEQ ID NO. 72) | C (SEQ ID NO: 167) |
| FCS variant F26 | E-R-R-K-R-S-V-D-T | GAGAGACGGAAACGCAGCGTTGACAC |
| | (SEQ ID NO. 73) | C (SEQ ID NO: 168) |
| FCS variant F27 | Q-R-R-K-R-S-V-D-T | CAGAGACGGAAACGCAGCGTTGACAC |
| | (SEQ ID NO. 74) | C (SEQ ID NO: 169) |
| FCS variant F28 | H-R-R-K-R-S-V-D-T | CACAGACGGAAACGCAGCGTTGACAC |
| | (SEQ ID NO. 75) | C (SEQ ID NO: 170) |
| FCS variant F29 | I-R-R-K-R-S-V-D-T | ATCAGACGGAAACGCAGCGTTGACAC |
| | (SEQ ID NO. 76) | C (SEQ ID NO: 171) |
| FCS variant F30 | L-R-R-K-R-S-V-D-T | CTGAGACGGAAACGCAGCGTTGACAC |
| | (SEQ ID NO. 77) | C (SEQ ID NO: 172) |
| FCS variant F31 | M-R-R-K-R-S-V-D-T | ATGAGACGGAAACGCAGCGTTGACAC |
| | (SEQ ID NO. 78) | C (SEQ ID NO: 173) |
| FCS variant F32 | F-R-R-K-R-S-V-D-T | TTCAGACGGAAACGCAGCGTTGACAC |
| | (SEQ ID NO. 79) | C (SEQ ID NO: 174) |
| FCS variant F33 | S-R-R-K-R-S-V-D-T | TCCAGACGGAAACGCAGCGTTGACAC |
| | (SEQ ID NO. 80) | C (SEQ ID NO: 175) |
| FCS variant F34 | T-R-R-K-R-S-V-D-T | ACCAGACGGAAACGCAGCGTTGACAC |
| | (SEQ ID NO. 81) | C (SEQ ID NO: 176) |
| FCS variant F35 | W-R-R-K-R-S-V-D-T | TGGAGACGGAAACGCAGCGTTGACAC |
| | (SEQ ID NO. 82) | C (SEQ ID NO: 177) |
| FCS variant F36 | Y-R-R-K-R-S-V-D-T | TACAGACGGAAACGCAGCGTTGACAC |
| | (SEQ ID NO. 83) | C (SEQ ID NO: 178) |
| FCS variant F37 | V-R-R-K-R-S-V-D-T | GTGAGACGGAAACGCAGCGTTGACAC |
| | (SEQ ID NO. 84) | C (SEQ ID NO: 179) |
| FCS variant F38 | R-R-R-K-R-P-V-D-T | AGAAGACGGAAACGCCCTGTTGACAC |
| | (SEQ ID NO. 85) | C (SEQ ID NO: 180) |
| FCS variant F39 | R-R-R-K-R-G-V-D-T | AGAAGACGGAAACGCGGCGTTGACAC |
| | (SEQ ID NO. 86) | C (SEQ ID NO: 181) |
| FCS variant F40 | R-R-R-K-R-A-V-D-T | AGAAGACGGAAACGCGCCGTTGACAC |
| | (SEQ ID NO. 87) | C (SEQ ID NO: 182) |
| FCS variant F41 | R-R-R-K-R-R-V-D-T | AGAAGACGGAAACGCAGAGTTGACAC |
| | (SEQ ID NO. 88) | C (SEQ ID NO: 183) |
| FCS variant F42 | R-R-R-K-R-K-V-D-T | AGAAGACGGAAACGCAAGGTTGACAC |
| | (SEQ ID NO. 89) | C (SEQ ID NO: 184) |
| FCS variant F43 | R-R-R-K-R-N-V-D-T | AGAAGACGGAAACGCAACGTTGACAC |
| | (SEQ ID NO. 90) | C (SEQ ID NO: 185) |
| FCS variant F44 | R-R-R-K-R-D-V-D-T | AGAAGACGGAAACGCGACGTTGACAC |
| | (SEQ ID NO. 91) | C (SEQ ID NO: 186) |
| FCS variant F45 | R-R-R-K-R-C-V-D-T | AGAAGACGGAAACGCTGCGTTGACAC |
| | (SEQ ID NO. 92) | C (SEQ ID NO: 187) |
| FCS variant F46 | R-R-R-K-R-E-V-D-T | AGAAGACGGAAACGCGAGGTTGACAC |
| | (SEQ ID NO. 93) | C (SEQ ID NO: 188) |
| FCS variant F47 | R-R-R-K-R-Q-V-D-T | AGAAGACGGAAACGCCAGGTTGACAC |
| | (SEQ ID NO. 94) | C (SEQ ID NO: 189) |
| FCS variant F48 | R-R-R-K-R-H-V-D-T | AGAAGACGGAAACGCCACGTTGACAC |
| | (SEQ ID NO. 95) | C (SEQ ID NO: 190) |
| FCS variant F49 | R-R-R-K-R-I-V-D-T | AGAAGACGGAAACGCATCGTTGACAC |
| | (SEQ ID NO. 96) | C (SEQ ID NO: 191) |
| FCS variant F50 | R-R-R-K-R-L-V-D-T | AGAAGACGGAAACGCCTCGTTGACAC |
| | (SEQ ID NO. 97) | C (SEQ ID NO: 192) |
| FCS variant F51 | R-R-R-K-R-M-V-D-T | AGAAGACGGAAACGCATGGTTGACAC |
| | (SEQ ID NO. 98) | C (SEQ ID NO: 193) |
| FCS variant F52 | R-R-R-K-R-F-V-D-T | AGAAGACGGAAACGCTTCGTTGACACC |
| | (SEQ ID NO. 99) | (SEQ ID NO: 194) |
| FCS variant F53 | R-R-R-K-R-T-V-D-T | AGAAGACGGAAACGCACCGTTGACAC |
| | (SEQ ID NO. 100) | C (SEQ ID NO: 195) |
| FCS variant F54 | R-R-R-K-R-W-V-D-T | AGAAGACGGAAACGCTGGGTTGACAC |
| | (SEQ ID NO. 101) | C (SEQ ID NO: 196) |
| FCS variant F55 | R-R-R-K-R-Y-V-D-T | AGAAGACGGAAACGCTACGTTGACAC |
| | (SEQ ID NO. 102) | C (SEQ ID NO: 197) |
| FCS variant F56 | R-R-R-K-R-V-V-D-T | AGAAGACGGAAACGCGTTGTTGACAC |
| | (SEQ ID NO: 103) | C (SEQ ID NO: 198) |
| FCS variant F57 | R-R-R-K-R-S-P-D-T | AGAAGACGGAAACGCAGCCCTGACAC |
| | (SEQ ID NO: 104) | C (SEQ ID NO: 199) |
| FCS variant F58 | R-R-R-K-R-S-G-D-T | AGAAGACGGAAACGCAGCGGCGACAC |
| | (SEQ ID NO: 105) | C (SEQ ID NO: 200) |
| FCS variant F59 | R-R-R-K-R-S-A-D-T | AGAAGACGGAAACGCAGCGCCGACAC |
| | (SEQ ID NO: 106) | C (SEQ ID NO: 201) |
| FCS variant F60 | R-R-R-K-R-S-R-D-T | AGAAGACGGAAACGCAGCAGAGACAC |
| | (SEQ ID NO: 107) | C (SEQ ID NO: 202) |
| FCS variant F61 | R-R-R-K-R-S-K-D-T | AGAAGACGGAAACGCAGCAAGGACAC |
| | (SEQ ID NO: 108) | C (SEQ ID NO: 203) |
| FCS variant F62 | R-R-R-K-R-S-N-D-T | AGAAGACGGAAACGCAGCAACGACAC |
| | (SEQ ID NO: 109) | C (SEQ ID NO: 204) |
| FCS variant F63 | R-R-R-K-R-S-D-D-T | AGAAGACGGAAACGCAGCGACGACAC |
| | (SEQ ID NO: 110) | C (SEQ ID NO: 205) |
| FCS variant F64 | R-R-R-K-R-S-C-D-T | AGAAGACGGAAACGCAGCTGCGACAC |
| | (SEQ ID NO: 111) | C (SEQ ID NO: 206) |
| FCS variant F65 | R-R-R-K-R-S-E-D-T | AGAAGACGGAAACGCAGCGAGGACAC |
| | (SEQ ID NO: 112) | C (SEQ ID NO: 207) |
| FCS variant F66 | R-R-R-K-R-S-Q-D-T | AGAAGACGGAAACGCAGCCAGGACAC |
| | (SEQ ID NO: 113) | C (SEQ ID NO: 208) |
| FCS variant F67 | R-R-R-K-R-S-H-D-T | AGAAGACGGAAACGCAGCCACGACAC |
| | (SEQ ID NO: 114) | C (SEQ ID NO: 209) |
| FCS variant F68 | R-R-R-K-R-S-I-D-T | AGAAGACGGAAACGCAGCATCGACAC |
| | (SEQ ID NO: 115) | C (SEQ ID NO: 210) |
| FCS variant F69 | R-R-R-K-R-S-L-D-T | AGAAGACGGAAACGCAGCCTCGACAC |
| | (SEQ ID NO: 116) | C (SEQ ID NO: 211) |
| FCS variant F70 | R-R-R-K-R-S-M-D-T | AGAAGACGGAAACGCAGCATGGACAC |
| | (SEQ ID NO: 117) | C (SEQ ID NO: 212) |
| FCS variant F71 | R-R-R-K-R-S-F-D-T | AGAAGACGGAAACGCAGCTTCGACAC |
| | (SEQ ID NO: 118) | C (SEQ ID NO: 213) |
| FCS variant F72 | R-R-R-K-R-S-S-D-T | AGAAGACGGAAACGCAGCAGCGACAC |
| | (SEQ ID NO: 119) | C (SEQ ID NO: 214) |
| FCS variant F73 | R-R-R-K-R-S-T-D-T | AGAAGACGGAAACGCAGCACCGACAC |
| | (SEQ ID NO: 120) | C (SEQ ID NO: 215) |
| FCS variant F74 | R-R-R-K-R-S-W-D-T | AGAAGACGGAAACGCAGCTGGGACAC |
| | (SEQ ID NO: 121) | C (SEQ ID NO: 216) |
| FCS variant F75 | R-R-R-K-R-S-Y-D-T | AGAAGACGGAAACGCAGCTACGACAC |
| | (SEQ ID NO: 122) | C (SEQ ID NO: 217) |
| FCS variant F76 | R-R-R-K-R-S-V-P-T | AGAAGACGGAAACGCAGCGTTCCTAC |
| | (SEQ ID NO: 123) | C (SEQ ID NO: 218) |
| FCS variant F77 | R-R-R-K-R-S-V-G-T | AGAAGACGGAAACGCAGCGTTGGCAC |
| | (SEQ ID NO: 124) | C (SEQ ID NO: 219) |
| FCS variant F78 | R-R-R-K-R-S-V-A-T | AGAAGACGGAAACGCAGCGTTGCCAC |
| | (SEQ ID NO: 125) | C (SEQ ID NO: 220) |
| FCS variant F79 | R-R-R-K-R-S-V-R-T | AGAAGACGGAAACGCAGCGTTAGAAC |
| | (SEQ ID NO: 126) | C (SEQ ID NO: 221) |
| FCS variant F80 | R-R-R-K-R-S-V-K-T | AGAAGACGGAAACGCAGCGTTAAGAC |
| | (SEQ ID NO: 127) | C (SEQ ID NO: 222) |
| FCS variant F81 | R-R-R-K-R-S-V-N-T | AGAAGACGGAAACGCAGCGTTAACAC |
| | (SEQ ID NO: 128) | C (SEQ ID NO: 223) |
| FCS variant F82 | R-R-R-K-R-S-V-C-T | AGAAGACGGAAACGCAGCGTTTGCAC |
| | (SEQ ID NO: 129) | C (SEQ ID NO: 224) |
| FCS variant F83 | R-R-R-K-R-S-V-E-T | AGAAGACGGAAACGCAGCGTTGAGAC |
| | (SEQ ID NO: 130) | C (SEQ ID NO: 225) |
| FCS variant F84 | R-R-R-K-R-S-V-Q-T | AGAAGACGGAAACGCAGCGTTCAGAC |
| | (SEQ ID NO: 131) | C (SEQ ID NO: 226) |
| FCS variant F85 | R-R-R-K-R-S-V-H-T | AGAAGACGGAAACGCAGCGTTCACAC |
| | (SEQ ID NO: 132) | C (SEQ ID NO: 227) |
| FCS variant F86 | R-R-R-K-R-S-V-I-T | AGAAGACGGAAACGCAGCGTTATCAC |
| | (SEQ ID NO: 133) | C (SEQ ID NO: 228) |
| FCS variant F87 | R-R-R-K-R-S-V-L-T | AGAAGACGGAAACGCAGCGTTCTCAC |
| | (SEQ ID NO: 134) | C (SEQ ID NO: 229) |
| FCS variant F88 | R-R-R-K-R-S-V-M-T | AGAAGACGGAAACGCAGCGTTATGAC |
| | (SEQ ID NO: 135) | C (SEQ ID NO: 230) |
| FCS variant F89 | R-R-R-K-R-S-V-F-T | AGAAGACGGAAACGCAGCGTTTTCACC |
| | (SEQ ID NO: 136) | (SEQ ID NO: 231) |
| FCS variant F90 | R-R-R-K-R-S-V-S-T | AGAAGACGGAAACGCAGCGTTAGCAC |
| | (SEQ ID NO: 137) | C (SEQ ID NO: 232) |
| FCS variant F91 | R-R-R-K-R-S-V-T-T | AGAAGACGGAAACGCAGCGTTACCAC |
| | (SEQ ID NO: 138) | C (SEQ ID NO: 233) |
| FCS variant F92 | R-R-R-K-R-S-V-W-T | AGAAGACGGAAACGCAGCGTTTGGAC |
| | (SEQ ID NO: 139) | C (SEQ ID NO: 234) |
| FCS variant F93 | R-R-R-K-R-S-V-Y-T | AGAAGACGGAAACGCAGCGTTTACAC |
| | (SEQ ID NO: 140) | C (SEQ ID NO: 235) |
| FCS variant F94 | R-R-R-K-R-S-V-V-T | AGAAGACGGAAACGCAGCGTTGTTAC |
| | (SEQ ID NO: 141) | C (SEQ ID NO: 236) |
| FCS variant F95 | R-R-R-K-R-S-V-D-P | AGAAGACGGAAACGCAGCGTTGACCC |
| | (SEQ ID NO: 142) | T (SEQ ID NO: 237) |
| FCS variant F96 | R-R-R-K-R-S-V-D-G | AGAAGACGGAAACGCAGCGTTGACGG |
| | (SEQ ID NO: 143) | C (SEQ ID NO: 238) |
| FCS variant F97 | R-R-R-K-R-S-V-D-A | AGAAGACGGAAACGCAGCGTTGACGC |
| | (SEQ ID NO: 144) | C (SEQ ID NO: 239) |
| FCS variant F98 | R-R-R-K-R-S-V-D-R | AGAAGACGGAAACGCAGCGTTGACAG |
| | (SEQ ID NO: 145) | A (SEQ ID NO: 240) |
| FCS variant F99 | R-R-R-K-R-S-V-D-K | AGAAGACGGAAACGCAGCGTTGACAA |
| | (SEQ ID NO: 146) | G (SEQ ID NO: 241) |
| FCS variant F100 | R-R-R-K-R-S-V-D-N | AGAAGACGGAAACGCAGCGTTGACAA |
| | (SEQ ID NO: 147) | C (SEQ ID NO: 242) |
| FCS variant F101 | R-R-R-K-R-S-V-D-D | AGAAGACGGAAACGCAGCGTTGACGA |
| | (SEQ ID NO: 148) | C (SEQ ID NO: 243) |
| FCS variant F102 | R-R-R-K-R-S-V-D-C | AGAAGACGGAAACGCAGCGTTGACTG |
| | (SEQ ID NO: 149) | C (SEQ ID NO: 244) |
| FCS variant F103 | R-R-R-K-R-S-V-D-E | AGAAGACGGAAACGCAGCGTTGACGA |
| | (SEQ ID NO: 150) | G (SEQ ID NO: 245) |
| FCS variant F104 | R-R-R-K-R-S-V-D-Q | AGAAGACGGAAACGCAGCGTTGACCA |
| | (SEQ ID NO: 151) | G (SEQ ID NO: 246) |
| FCS variant F105 | R-R-R-K-R-S-V-D-H | AGAAGACGGAAACGCAGCGTTGACCA |
| | (SEQ ID NO: 152) | C (SEQ ID NO: 247) |
| FCS variant F106 | R-R-R-K-R-S-V-D-I | AGAAGACGGAAACGCAGCGTTGACAT |
| | (SEQ ID NO: 153) | C (SEQ ID NO: 248) |
| FCS variant F107 | R-R-R-K-R-S-V-D-L | AGAAGACGGAAACGCAGCGTTGACCT |
| | (SEQ ID NO: 154) | C (SEQ ID NO: 249) |
| FCS variant F108 | R-R-R-K-R-S-V-D-M | AGAAGACGGAAACGCAGCGTTGACAT |
| | (SEQ ID NO: 155) | G (SEQ ID NO: 250) |
| FCS variant F109 | R-R-R-K-R-S-V-D-F | AGAAGACGGAAACGCAGCGTTGACTT |
| | (SEQ ID NO: 156) | C (SEQ ID NO: 251) |
| FCS variant F110 | R-R-R-K-R-S-V-D-S | AGAAGACGGAAACGCAGCGTTGACAG |
| | (SEQ ID NO: 157) | C (SEQ ID NO: 252) |
| FCS variant F111 | R-R-R-K-R-S-V-D-W | AGAAGACGGAAACGCAGCGTTGACTG |
| | (SEQ ID NO: 158) | G (SEQ ID NO: 253) |
| FCS variant F112 | R-R-R-K-R-S-V-D-Y | AGAAGACGGAAACGCAGCGTTGACTA |
| | (SEQ ID NO: 159) | C (SEQ ID NO: 254) |
| FCS variant F113 | R-R-R-K-R-S-V-D-V | AGAAGACGGAAACGCAGCGTTGACGT |
| | (SEQ ID NO: 160) | T (SEQ ID NO: 255) |
| Alternative | Z-R-X1-X2-R-X3-X4- | |
| general sequence | X5-X6 (SEQ ID NO: | |
| of a first cleavage | 256) | |
| site | | |

1. An expression system for an antigen binding molecule, comprising:
a first polynucleotide encoding a first part of the antigen binding molecule;
a second polynucleotide encoding a first cleavage site comprising an amino acid sequence Z-R-X1-X2-R-X3-X4-X5-X6 (SEQ ID NO: 256), wherein the second polynucleotide is located downstream of the first polynucleotide;
a third polynucleotide encoding a second cleavage site comprising a 2A polypeptide or a fragment thereof, wherein the third polynucleotide is located downstream of the second polynucleotide; and
a fourth polynucleotide encoding a second part of the antigen binding molecule, wherein the fourth polynucleotide is located downstream of the third polynucleotide,
wherein when the first and second cleavage sites are cleaved, an antigen binding molecule comprising the first and second parts of the antigen binding molecule is released,
wherein Z is R, P, G, A, K, N, Q, H, I, L, M, F, S, T, W, Y, or V, and
wherein X1, X2, X3, X4, X5 and X6 is R, P, G, A, D, C, E, K, N, Q, H, I, L, M, F, S, T, W, Y, or V.

2. The expression of claim 1, further comprising:
a fifth polynucleotide encoding a third cleavage site comprising a Furin cleavage sequence variant comprising:
(i) a Furin consensus sequence R-X-K-R (SEQ ID NO: 18); or
(ii) a Furin consensus sequence R-X-R-R (SEQ ID NO: 19); or
(iii) a Furin consensus sequence R-X-K-R (SEQ ID NO: 18) and at least the first five amino acids derived from a 2A polypeptide; or
(iv) a Furin consensus sequence R-X-R-R (SEQ ID NO: 19) and at least the first five amino acids derived from a 2A polypeptide; or
(v) a Furin consensus sequence R-X-K-R (SEQ ID NO: 18) and the first five amino acids derived from a 2A polypeptide, wherein the first five amino acids derived from the 2A polypeptide comprise one or more point mutations; or
(vi) a Furin consensus sequence R-X-R-R (SEQ ID NO: 19) and the first five amino acids derived from a 2A polypeptide, wherein the first five amino acids derived from the 2A polypeptide comprise one or more point mutations; or
(vii) a Furin consensus sequence R-X-K-R (SEQ ID NO: 18) and the first five amino acids derived from a 2A polypeptide, wherein the first five amino acids derived from the 2A polypeptide comprise one or more point mutations selected from the group consisting of A1P, A1G, T2G, T2P, N3P, N3A, F4P, F4A and S5P; or
(viii) a Furin consensus sequence R-X-R-R (SEQ ID NO: 19) and the first five amino acids derived from a 2A polypeptide, wherein the first five amino acids derived from the 2A polypeptide comprise one or more point mutations selected from the group consisting of A1P, A1G, T2G, T2P, N3P, N3A, F4P, F4A and S5P;
wherein the fifth polynucleotide is located downstream of the fourth polynucleotide; and
a sixth polynucleotide encoding a membrane anchor polypeptide, wherein the sixth polynucleotide is located downstream of the fifth polynucleotide;
wherein the antigen binding molecule is either secretable or membrane-bound,
wherein when the first, second, and third cleavage sites are cleaved, a secretable antigen binding molecule comprising the first and second parts of the antigen binding molecule is released;
wherein when the first and second cleavage sites are cleaved and the third cleavage site is not cleaved, a membrane-bound antigen binding molecule comprising the first and second parts of the antigen binding molecule, the third cleavage site, and the membrane anchor polypeptide is released, and
wherein X is any amino acid.

3. The expression system of claim 1, wherein;
(i) Z is R and the amino acid sequence of the first cleavage site comprises an amino acid sequence R-R-X1-X2-R-X3-X4-X5-X6 (SEQ ID NO: 1); or
(ii) X1 is R, X2 is K or R, and X3, X4, X5, and X6 is any one of P, G, A, D, C, E, K, N, Q, H, I, L, M, F, S, T, W, Y, or V; or
(iii) X3 is any one of P, G, A, D, E, H, I, L, M, F, or S; X4 is any one of P, A, I, L, T, or V; X5 is any one of R, P, G, A, D, C, E, K, N, Q, H, I, L, M, F, S, T, W, Y, or V; and X6 is any one of R, P, G, A, D, C, E, K, N, Q, H, I, L, M, F, S, T, W, Y, or V; or
(iv) Z is R, P, G, A, K, N, Q, H, I, L, M, F, S, T, W, Y, or V; X3 is any one of S, A, D, or E; X4 is any one of V, A, I, L or T; X5 is any one of R, P, G, A, D, C, E, K, N, Q, H, I, L, M, F, S, T, W, Y, or V; and X6 is any one of R, P, G, A, D, C, E, K, N, Q, H, I, L, M, F, S, T, W, Y, or V.

4. (canceled)

5. The expression system of claim 1, wherein the first cleavage site comprises an amino acid sequence set forth in SEQ ID NOs: 2-17 and SEQ ID NOs: 66-160, wherein optionally the first cleavage site comprises an amino acid sequence set forth in SEQ ID NOs: 2-17, 66-70, 74-84, 86, 87, 91, 93, 95-99, 104, 106, 115, 116, 120, 123-129, and 130-160, and wherein further optionally the first cleavage site comprises an amino acid sequence set forth in SEQ ID Nos 2, 87, 91, 93, 106, 115, 116, and 120.

6.-9. (canceled)

10. The expression system of claim 1, further comprising a polynucleotide encoding a linker SGSG between the second and third polynucleotides.

11.-13. (canceled)

14. The expression system of claim 1, wherein the 2A polypeptide is selected from a group consisting of P2A, F2A, E2A, and T2A, wherein optionally the 2A polypeptide is T2A.

15. The expression system of claim 1, wherein the membrane anchor polypeptide is glycophospholipid transmembrane domain (GPI), platelet-derived growth factor receptor (PDGFR) beta chain transmembrane domain (PTM), or immunoglobulin C2-type extracellular-transmembrane-cytosolic domain or murin B7-1 antigen, wherein optionally the membrane anchor polypeptide is GPI.

16. The expression system of claim 1, wherein the second polynucleotide encodes a first cleavage site having a higher cleavage efficiency of at least 80%, 81%, 82%, 83%, 84%, 85%, 86%, 87%, 88%, 89%, 90%, 91%, 92%, 93%, 94%, 95%, 96%, 97%, 98%, or 99% compared to an expression system comprising a second polynucleotide that encodes a first cleavage site comprising a Furin consensus sequence R-X-K-R (SEQ ID NO: 18) or R-X-R-R (SEQ ID NO: 19).

17. The expression system of claim 1, wherein the expression system is capable of releasing an antigen binding molecule comprising a first part of the antigen binding molecule having the correct polypeptide sequence, wherein the first part of the antigen binding molecule does not contain one or more residual amino acids from the second cleavage site after the cleavage of the first and second cleavage sites.

18. The expression system of claim 1, wherein the expression system is capable of improving the homogeneity of the antigen binding molecules produced, wherein homogeneity is achieved when a plurality of antigen binding molecules are produced, wherein each antigen binding molecule in the plurality of antigen binding molecules comprises a first part having the same amino acid sequence and/or molecular weight.

19. The expression system of claim 1, wherein the expression system is delivered to a target cell prior to transcription, wherein optionally the target cell is an animal cell, and wherein optionally the animal cell is a mammalian cell.

20. The expression system of claim 1, wherein the second polynucleotide comprises a nucleotide sequence set forth in SEQ ID NOs: 35-50 and SEQ ID NOs: 161-255.

21. A vector comprising the expression system of claim 1.

22. A host cell comprising the expression system of claim 1.

23. A kit comprising the expression system of the vector of claim 21.

24. The expression system of claim 1, for use in screening antibody libraries or antibody production.

25. A method of producing one or more secretable antigen binding molecules and/or one or more membrane-bound antigen binding molecules, comprising:
(i) culturing the host cell of claim 22 under suitable culture conditions such that one or more secretable antigen binding molecules and/or one or more membrane-bound antigen binding molecules are produced, or
(ii) culturing the host cell of claim 22 under suitable culture conditions such that one or more secretable antigen binding molecules and/or one or more membrane-bound antigen binding molecules are produced and recovering the one or more secretable antigen binding molecules.

26. (canceled)

27. A method for detecting the presence of one or more secretable antigen binding molecules and/or one or more membrane-bound antigen binding molecules, the method comprising:
providing an expression system of claim 1;
delivering said expression system to one or more target cells;
wherein said target cells transcribe said expression system into one or more amino acid sequences,
wherein when all of the cleavage sites in the one or more amino acid sequences are cleaved, one or more secretable antigen binding molecules comprising the first and second parts of the antigen binding molecules are secreted by the target cells,
wherein when the first and second cleavage sites of the one or more amino acid sequences are cleaved and the third cleavage site of the one or more amino acid sequences is not cleaved, one or more membrane-bound antigen binding molecules comprising the first and second parts of the antigen binding molecules are bound to the surface of the target cells; and
detecting the presence or absence of the one or more secretable antigen binding molecules and/or one or more membrane-bound antigen binding molecules.

28. The method of claim 27, wherein:
(i) the presence or absence of the one or more secretable antigen binding molecules is detected using enzyme-linked immunosorbent assay (ELISA) or alternative binding assay; and/or
(ii) the presence or absence of the one or more membrane-bound antigen binding molecules is detected using flow cytometry analysis, wherein the first part of the antigen binding molecules, the second part of the antigen binding molecules and/or an antigen specific to the antigen binding molecules are stained prior to the flow cytometry analysis.

29. A host cell comprising the vector of claim 21.

30. A kit comprising the host cell of claim 22.

31. The host cell of claim 22 for use in screening antibody libraries or antibody production.

32. The kit of claim 23 for use in screening antibody libraries or antibody production.
